(19) United States
(12) Patent Application Publication
(10) Pub. No.: US 2009/0192169 A1
(43) Pub. Date: Jul. 30, 2009

(54) Bicyclic Benzimidazole Compounds and Their Use as Metabotropic Glutamate Receptor Potentiators

(76) Inventors: Ian Egle, Georgetown (CA); Methvin Isaac, Brampton (CA); Rebecca Urbanek, Wilmington, DE (US); Frances M. McLaren, Wilmington, DE (US); Sally B. Walsh, Wilmington, DE (US); Gary B. Steelman, Wilmington, DE (US); Dean G. Brown, Wilmington, DE (US); David Nugiel, Wilmington, DE (US); Deborah W. Chen, Singapore (SG); Abdelmalik Slassi, Mississauga (CA); Fupeng Ma, Melrose, MA (US)

Correspondence Address: BIRCH, STEWART, KOLASCH & BIRCH, LLP, P.O. BOX 747, FALLS CHURCH, VA, 22040-0747, US

(21) Appl. No.: 12/225,310

(22) PCT Filed: Mar. 29, 2007
(86) PCT No.: PCT/US2007/065469

§ 371 Date: Dec. 24, 2008

Related U.S. Application Data
(60) Provisional application No. 60/787,634, filed on Mar. 31, 2006.

(51) Int. Cl. A61K 31/454; C07D 401/06; C07D 403/06; A61P 35/00; A61K 31/496; A61K 31/4184

(52) U.S. Cl. 514/254.06; 546/199; 544/370; 548/306.1; 514/322; 514/394

(57) ABSTRACT

Compounds of Formula I:

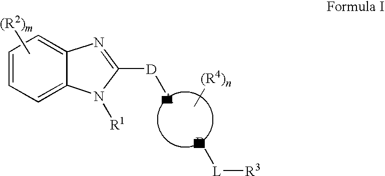

wherein A, B, D, L, R¹, R², R³, R⁴, m, and n are as defined for Formula I in the description. The invention also relates to processes for the preparation of the compounds and to new intermediates employed in the preparation, pharmaceutical compositions containing the compounds, and to the use of the compounds in therapy.

### BACKGROUND OF THE INVENTION

The present invention relates to novel compounds which are potentiators of glutamate receptors, methods for their preparation, pharmaceutical compositions containing them and their use in therapy.

The metabotropic glutamate receptors (mGluR) constitute a family of GTP-binding-protein (G-protein) coupled receptors that are activated by glutamate, and have important roles in synaptic activity in the central nervous system, including neural plasticity, neural development and neurodegeneration.

Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels (Schoepp et al., 1993, Trends Pharmacol. Sci., 14:13; Schoepp, 1994, Neurochem. Int., 24:439; Pin et al., 1995, Neuropharmacology 34:1; Bordi & Ugolini, 1999, Prog. Neurobiol. 59:55).

Eight mGluR subtypes have been identified, which are divided into three groups based upon primary sequence similarity, signal transduction linkages, and pharmacological profile. Group-I includes mGluR1 and mGluR5, which activate phospholipase C and the generation of an intracellular calcium signal. The Group-II (mGluR2 and mGluR3) and Group-III (mGluR4, mGluR6, mGluR7, and mGluR8) mGluRs mediate an inhibition of adenylyl cyclase activity and cyclic AMP levels. For a review, see Pin et al., 1999, Eur. J. Pharmacol., 375:277-294.

Members of the mGluR family of receptors are implicated in a number of normal processes in the mammalian CNS, and are important targets for compounds for the treatment of a variety of neurological and psychiatric disorders. Activation of mGluRs is required for induction of hippocampal long-term potentiation and cerebellar long-term depression (Bashir et al., 1993, Nature, 363:347; Bortolotto et al., 1994, Nature, 368:740; Aiba et al., 1994, Cell, 79:365; Aiba et al., 1994, Cell, 79:377). A role for mGluR activation in nociception and analgesia also has been demonstrated (Meller et al., 1993, Neuroreport, 4: 879; Bordi & Ugolini, 1999, Brain Res., 871:223). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex (Nakanishi, 1994, Neuron, 13:1031; Pin et al., 1995, Neuropharmacology, supra; Knopfel et al., 1995, J. Med. Chem., 38:1417).

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

### SUMMARY OF THE INVENTION

The invention satisfies the need for new drugs and compounds that can modulate mGluR function and others by providing, as one object, compounds of Formula I,

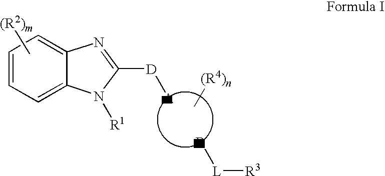

wherein,
A and B are independently selected from the group consisting of N and C, with the proviso that A and B are not both C;

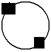

represents a 4- to 8-membered ring;
D is selected from the group consisting of alkylene, alkenylene, and alkynylene;
L is selected from the group consisting of a bond, alkylene, alkenylene, alkynylene, —O—, —X—O—, —O—X—, —X—O—Y, —NR¹⁰—, —X—NR¹⁰—, —NR¹⁰—X—, and —X—NR¹⁰—Y—; wherein X and Y, in each instance, are independently selected from the group consisting of alkylene, alkenylene, and alkynylene, with the proviso that when B is N, L is selected from the group consisting of a bond, alkylene, alkenylene, alkynylene, —X—O—, —X—O—Y—, —X—NR¹⁰—, and —X—NR¹⁰—Y—;
R¹ is selected from the group consisting of hydrogen, alkyl, alkylhalo, alkenyl, alkenylhalo, alkynyl, alkynylhalo, cycloalkyl, heterocycloalkyl, aryl, heteroaryl, alkylene-cycloalkyl, alkenylene-cycloalkyl, alkynylene-cycloalkyl, alkylene-heterocycloalkyl, alkenylene-heterocycloalkyl, alkynylene-heterocycloalkyl, alkylene-aryl, alkenylene-aryl, alkynylene-aryl, alkylene-heteroaryl, alkenylene-heteroaryl, alkynylene-heteroaryl, alkylene-OR⁷, alkenylene-OR⁷, alkynylene-OR⁷, alkylene-NR⁸R⁹, alkenylene-NR⁸R⁹, alkynylene-NR⁸R⁹, alkylene-cyano, alkenylene-cyano, alkynylene-cyano, alkylene-(CO)R⁷, alkenylene-(CO)R⁷, and alkynylene-(CO)R⁷; wherein any cyclic group is optionally substituted by one or more substituents independently selected from the group consisting of halogen, alkyl, —O-alkyl, alkylhalo, and —O-alkylhalo;
R², in each instance, is independently selected from the group consisting of hydrogen, halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, and —O-alkynylene-heteroaryl; wherein any cyclic group is optionally substituted by one or more substituents independently selected from the group consisting of halogen, alkyl, —O-alkyl, alkylhalo, and —O-alkylhalo;
R³ is selected from the group consisting of hydrogen, aryl, heteroaryl, and benzo-cycloC₅₋₈alkenyl; wherein any carbocyclic group is optionally substituted by one or more independently selected substituents, R⁵, and any heterocyclic group is optionally substituted by one or more independently selected substituents, R⁶;
R⁴, in each instance, is independently selected from the group consisting of hydrogen, halogen, hydroxyl, cyano, oxo, ═CR⁷R⁸, alkyl, alkylhalo, —O-alkyl, —O-alkylhalo, alkenyl, —O-alkenyl, alkynyl, —O-alkynyl, cycloalkyl, alkylene-cyclcoalkyl, heterocyloalkyl, alkylene-heterocycloalkyl, aryl, alkylene-aryl, heteroaryl, and alkylene-heteroaryl; wherein any cyclic group may be substituted by one or more substituents independently selected from the group consisting of halogen, alkyl, —O-alkyl, alkylhalo, and —O-alkylhalo;
R⁵, in each instance, is independently selected from the group consisting of halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, —O-alkynylene-heteroaryl, alkylene-cyano, —O-alkylene-cyano, alkenylene-cyano, —O-alkenylene-cyano, alkynylene-cyano, and —O-alkynylene-cyano; wherein any cyclic group is optionally substituted by one or more substituents independently selected from the group consisting of halogen, alkyl, —O-alkyl, alkylhalo, and —O-alkylhalo;
R⁶, in each instance, is independently selected from the group consisting of halogen, amino, cyano, alkyl, alkylhalo, alkenyl, alkynyl, and aryl; wherein said aryl is optionally substituted by one or more substituents independently selected from the group consisting of halogen, alkyl, —O-alkyl, alkylhalo, and —O-alkylhalo;
R⁷, R⁸, and R⁹ are independently selected from the group consisting of hydrogen, alkyl, alkylhalo, alkenyl, and alkynyl;
R¹⁰ is selected from the group consisting of hydrogen, alkyl, alkenyl, and alkynyl;
m represents an integer selected from the group consisting of 1, 2, 3, and 4; and n represents an integer selected from the group consisting of 1 and 2;
for use in the manufacture of a medicament for the therapy of neurological and psychiatric disorders associated with glutamate dysfunction.

Another object of the invention is to provide a pharmaceutical composition comprising a compound according to Formula I together with a pharmaceutically acceptable carrier or excipient.

Yet another object of the invention is a method for the treatment or prevention of neurological and psychiatric disorders associated with glutamate dysfunction in an animal in need of such treatment. The method comprises the step of administering to the animal a therapeutically effective amount of a compound of Formula I or a pharmaceutical composition thereof.

Another object of the invention provides a compound of Formula I, or a pharmaceutically acceptable salt or solvate thereof, for use in therapy.

Another object of the invention provides compounds of Formula II:

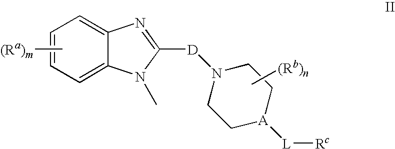

wherein:
A is selected from the group consisting of C and N;
D is an alkylene group;
L is selected from the group consisting of a bond, alkylene, alkylene-O—, —O-alkylene and alkylene-O-alkylene;
R^{a}, in each instance, is independently selected from the group consisting of halo and alkyl;

R^{b}, in each instance, is independently selected from the group consisting of halogen, cyano, oxo, hydroxy, alkyl, alkylhalo, —O-alkyl and —O-alkylhalo;

R^{c} is selected from the group consisting of aryl and heteroaryl, optionally substituted by one or more substituents independently selected from the group consisting of halo, cyano, hydroxy, alkyl, O-alkyl, alkylhalo, O-alkylhalo; and
m and n are independently selected from the group consisting of 0, 1, 2 and 3.

Another object of the invention is to provide a pharmaceutical composition comprising a compound according to Formula II together with a pharmaceutically acceptable carrier or excipient.

Yet another object of the invention is a method for the treatment or prevention of neurological and psychiatric disorders associated with glutamate dysfunction in an animal in need of such treatment. The method comprises the step of administering to the animal a therapeutically effective amount of a compound of Formula II or a pharmaceutical composition thereof.

Another object of the invention provides a compound of Formula II, or a pharmaceutically acceptable salt or solvate thereof, for use in therapy.

Still another object of the invention is the use of a compound according to Formula II, or a pharmaceutically acceptable salt or solvate thereof, for the manufacture of a medicament for the treatment of any of the conditions discussed herein.

The invention additionally provides processes for the preparation of compounds of Formulae I and II. General and specific processes are provided in more detail below.

### DETAILED DESCRIPTION OF THE PREFERRED EMBODIMENTS

The present invention is based upon the discovery of compounds that exhibit activity as pharmaceuticals, in particular as modulators of metabotropic glutamate receptors. More particularly, the compounds of the present invention exhibit activity as potentiators of the mGluR2 receptor, and are useful in therapy, in particular for the treatment of neurological and psychiatric disorders associated with glutamate dysfunction.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “C_{p-q}” used as a prefix, means any group having p to q carbon atoms, wherein p and q are 0 or positive integers, and q>p. For example, “C₁₋₆” would refer to a chemical group having 1 to 6 carbon atoms.

The term “alkyl” means a straight or branched hydrocarbon radical comprising 1 to 6 carbon atoms, and includes methyl, ethyl, propyl, isopropyl, t-butyl and the like.

The term “halo” means halogen and includes fluoro, chloro, bromo, iodo and the like, in both radioactive and non-radioactive forms.

The term “alkenyl” means a straight or branched hydrocarbon radical having at least one double bond and comprising 2 to 6 carbon atoms, and includes ethenyl, 1-propenyl, 1-butenyl and the like.

The term “alkynyl” means a straight or branched hydrocarbon radical having at least one triple bond and comprising 2 to 6 carbon atoms, and includes 1-propynyl (propargyl), 1-butynyl and the like.

The term “alkylhalo” means an alkyl radical substituted with one or more halogens on one or different carbons.

The term “alkenylhalo” means an alkenyl radical substituted with one or more halogens on one or different carbons.

The term “alkynylhalo” means an alkynyl radical substituted with one or more halogens on one or different carbons.

The term “alkylene” means a difunctional branched or unbranched saturated hydrocarbon radical having 1 to 6 carbon atoms, and includes methylene, ethylene, n-propylene, n-butylene and the like.

The term “alkenylene” means a difunctional branched or unbranched hydrocarbon radical having 2 to 6 carbon atoms and having at least one double bond, and includes ethenylene, n-propenylene, n-butenylene and the like.

The term “alkynylene” means a difunctional branched or unbranched hydrocarbon radical having 2 to 6 carbon atoms and having at least one triple bond, and includes ethynylene, n-propynylene, n-butynylene and the like.

The term “cycloalkyl” means a non-aromatic cyclic group (which may be unsaturated) having 3 to 7 carbon atoms, and includes cyclopropyl, cyclohexyl, cyclohexenyl and the like.

The term “heterocycloalkyl” means a 3- to 7-membered non-aromatic cyclic group (which may be unsaturated) having at least one heteroatom selected from the group consisting of N, S and O, and includes piperidinyl, piperazinyl, pyrrolidinyl, tetrahydrofuranyl and the like.

The term “aryl” means an aromatic group having 5 to 10 carbon atoms, and includes phenyl, naphthyl and the like.

The term “heteroaryl” means a 5- to 10-membered aromatic group which has at least one heteroatom selected from the group consisting of N, S, and O, and includes pyridyl, indolyl, furyl, benzofuryl, thienyl, benzothienyl, quinolyl, oxazolyl and the like.

The term “carbocyclic group” means an aromatic or non-aromatic cyclic group consisting of carbon atoms.

The term “heterocyclic group” means an aromatic or non-aromatic cyclic group including at least one heteroatom selected from the group consisting of N, S, and O.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

The term “solvate” means a compound of Formula I or the pharmaceutically acceptable salt of a compound of Formula I wherein molecules of a suitable solvent are incorporated into a crystal lattice. A suitable solvent is physiologically tolerable at the dosage administered as the solvate. Examples of suitable solvents are ethanol, water and the like. When water is the solvent, the molecule is referred to as a hydrate.

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

### Compounds

Compounds useful in the practice of the invention conform to Formula I:

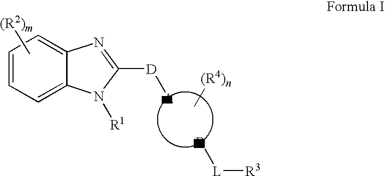

wherein,
A and B are independently selected from the group consisting of N and C, with the proviso that A and B are not both C;

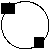

represents a 4- to 8-membered ring;
D is selected from the group consisting of alkylene, alkenylene, and alkynylene;
L is selected from the group consisting of a bond, alkylene, alkenylene, alkynylene, —O—, —X—O—, —O—X—, —X—O—Y, —NR¹⁰—, —X—NR¹⁰—, —NR¹⁰—X—, and —X—NR¹⁰—Y—; wherein X and Y, in each instance, are independently selected from the group consisting of alkylene, alkenylene, and alkynylene, with the proviso that when B is N, L is selected from the group consisting of a bond, alkylene, alkenylene, alkynylene, —X—O—, —X—O—Y—, —X—NR¹⁰—, and —X—NR¹⁰—Y—;
R¹ is selected from the group consisting of hydrogen, alkyl, alkylhalo, alkenyl, alkenylhalo, alkynyl, alkynylhalo, cycloalkyl, heterocycloalkyl, aryl, heteroaryl, alkylene-cycloalkyl, alkenylene-cycloalkyl, alkynylene-cycloalkyl, alkylene-heterocycloalkyl, alkenylene-heterocycloalkyl, alkynylene-heterocycloalkyl, alkylene-aryl, alkenylene-aryl, alkynylene-aryl, alkylene-heteroaryl, alkenylene-heteroaryl, alkynylene-heteroaryl, alkylene-OR⁷, alkenylene-OR⁷, alkynylene-OR⁷, alkylene-NR⁸R⁹, alkenylene-NR⁸R⁹, alkynylene-NR⁸R⁹, alkylene-cyano, alkenylene-cyano, alkynylene-cyano, alkylene-(CO)R⁷, alkenylene-(CO)R⁷, and alkynylene-(CO)R⁷; wherein any cyclic group is optionally substituted by one or more substituents independently selected from the group consisting of halogen, alkyl, —O-alkyl, alkylhalo, and —O-alkylhalo;
R², in each instance, is independently selected from the group consisting of hydrogen, halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, and —O-alkynylene-heteroaryl; wherein any cyclic group is optionally substituted by one or more substituents independently selected from the group consisting of halogen, alkyl, —O-alkyl, alkylhalo, and —O-alkylhalo;
R³ is selected from the group consisting of hydrogen, aryl, heteroaryl, and benzo-cycloC₅₋₈alkenyl; wherein any carbocyclic group is optionally substituted by one or more independently selected substituents, R⁵, and any heterocyclic group is optionally substituted by one or more independently selected substituents, R⁶;
R⁴, in each instance, is independently selected from the group consisting of hydrogen, halogen, hydroxyl, cyano, oxo, —CR⁷R⁸, alkyl, alkylhalo, —O-alkyl, —O-alkylhalo, alkenyl, —O-alkenyl, alkynyl, —O-alkynyl, cycloalkyl, alkylene-cyclcoalkyl, heterocyloalkyl, alkylene-heterocycloalkyl, aryl, alkylene-aryl, heteroaryl, and alkylene-heteroaryl; wherein any cyclic group may be substituted by one or more substituents independently selected from the group consisting of halogen, alkyl, —O-alkyl, alkylhalo, and —O-alkylhalo;
R⁵, in each instance, is independently selected from the group consisting of halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, —O-alkynylene-heteroaryl, alkylene-cyano, —O-alkylene-cyano, alkenylene-cyano, —O-alkenylene-cyano, alkynylene-cyano, and —O-alkynylene-cyano; wherein any cyclic group is optionally substituted by one or more substituents independently selected from the group consisting of halogen, alkyl, —O-alkyl, alkylhalo, and —O-alkylhalo;
R⁶, in each instance, is independently selected from the group consisting of halogen, amino, cyano, alkyl, alkylhalo, alkenyl, alkynyl, and aryl; wherein said aryl is optionally substituted by one or more substituents independently selected from the group consisting of halogen, alkyl, —O-alkyl, alkylhalo, and —O-alkylhalo;
R⁷, R⁸, and R⁹ are independently selected from the group consisting of hydrogen, alkyl, alkylhalo, alkenyl, and alkynyl;
R¹⁰ is selected from the group consisting of hydrogen, alkyl, alkenyl, and alkynyl;
m represents an integer selected from the group consisting of 1, 2, 3, and 4; and n represents an integer selected from the group consisting of 1 and 2.

Compounds of the invention further include compounds of Formula II:

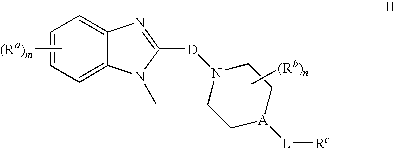

wherein:
A is selected from the group consisting of C and N;
D is an alkylene group;
L is selected from the group consisting of a bond, alkylene, alkylene-O—, —O-alkylene and alkylene-O-alkylene;
R^{a}, in each instance, is independently selected from the group consisting of halo and alkyl;

R^{b}, in each instance, is independently selected from the group consisting of halogen, cyano, oxo, hydroxy, alkyl, alkylhalo, —O-alkyl and —O-alkylhalo;

R^{c} is selected from the group consisting of aryl and heteroaryl, optionally substituted by one or more substituents independently selected from the group consisting of halo, cyano, hydroxy, alkyl, O-alkyl, alkylhalo, O-alkylhalo; and
m and n are independently selected from the group consisting of 0, 1, 2 and 3.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of Formula I. It will further be understood that the present invention encompasses tautomers of the compounds of Formula I.

It will also be understood by those of skill in the art that certain compounds of the present invention may exist in solvated, for example hydrated, as well as unsolvated forms. It will further be understood that the present invention encompasses all such solvated forms of the compounds of Formula I.

Within the scope of the invention are also salts of the compounds of Formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment of the present invention, the compound of Formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example | Structure | Name |
|---|---|---|
| 1.1 | 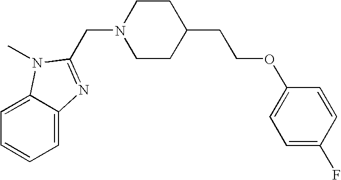 | 2-{4-[2-(4-Fluoro- phenoxy)-ethyl]- piperidin-1-ylmethyl}- 1-methyl-1H- benzoimidazole |
| 1.2 | 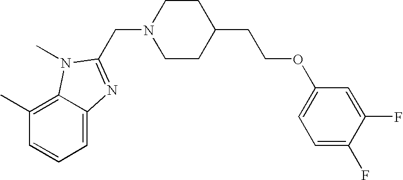 | 2-{4-[2-(3,4-Difluoro- phenoxy)-ethyl]- piperidin-1-ylmethyl}- 1,7-dimethyl-1H- benzoimidazole |
| 1.3 | 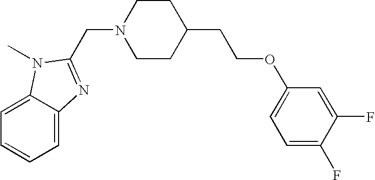 | 2-{4-[2-(3,4-Difluoro- phenoxy)-ethyl]- piperidin-1-ylmethyl}- 1-methyl-1H- benzoimidazole |
| 1.4 | 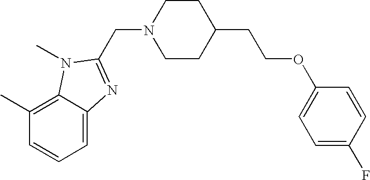 | 2-{4-[2-(4-Fluoro phenoxy)-ethyl]- piperidin-1-ylmethyl}- 1,7-dimethyl-1H- benzoimidazole |
| 1.5 | 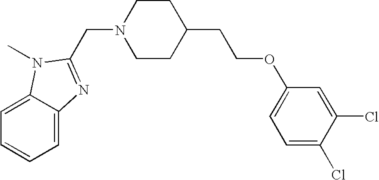 | 2-{4-[2-(3,4-Dichloro- phenoxy)-ethyl]- piperidin-1-ylmethyl}- 1-methyl-1H- benzoimidazole |
| 1.6 | 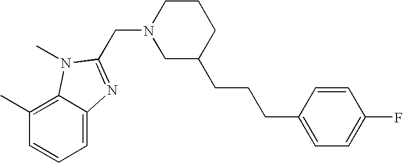 | 2-{3-[3-(4-Fluoro- phenyl)-propyl]- piperidin-1-ylmethyl}- 1,7-dimethyl-1H- benzoimidazole |
| 1.7 | 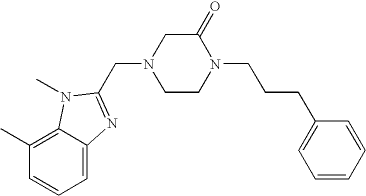 | 4-(1,7-Dimethyl-1H- benzoimidazol-2- ylmethyl)-1-(3-phenyl- propyl)-piperazin-2-one |
| 1.8 | 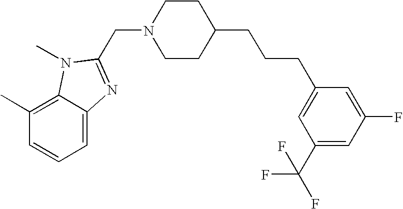 | 2-{4-[3-(3-Fluoro-5- trifluoromethyl-phenyl)- propyl]-piperidin-1- ylmethyl}-1,7-dimethyl- 1H-benzoimidazole |
| 1.9 | 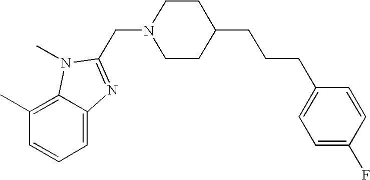 | 2-{4-[3-(4-Fluoro- phenyl)-propyl]- piperidin-1-ylmethyl}- 1,7-dimethyl-1H- benzoimidazole |
| 1.10 | 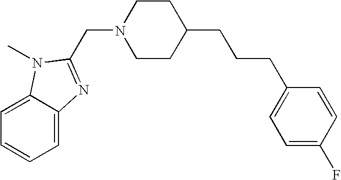 | 2-{4-[3-(4-Fluoro- phenyl)-propyl]- piperidin-1-ylmethyl}- 1-methyl-1H- benzoimidazole |
| 1.11 | 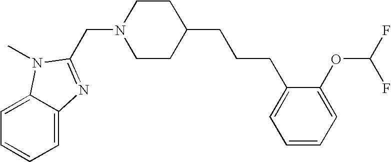 | 2-{4-[3-(2- Difluoromethoxy- phenyl)-propyl]- piperidin-1-ylmethyl}- 1-methyl-1H- benzoimidazole |
| 1.12 | 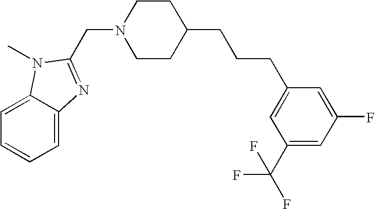 | 2-{4-[3-(3-Fluoro-5- trifluoromethyl-phenyl)- propyl]-piperidin-1- ylmethyl}-1-methyl-1H- benzoimidazole |
| 1.13 | 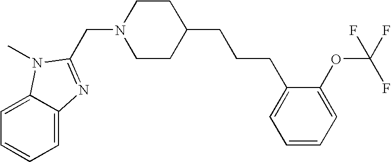 | 1-Methyl-2-{4-[3-(2- trifluoromethoxy- phenyl)-propyl]- piperidin-1-ylmethyl}- 1H-benzoimidazole |
| 1.14 | 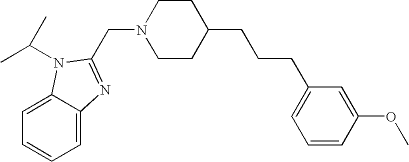 | 1-Isopropyl-2-{4-[3-(3- methoxy-phenyl)- propyl]-piperidin-1- ylmethyl}-1H- benzoimidazole |
| 1.15 | 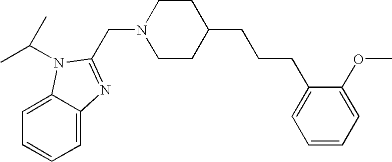 | 1-Isopropyl-2-{4-[3-(2- methoxy-phenyl)- propyl]-piperidin-1- ylmethyl}-1H- benzoimidazole |
| 1.16 | 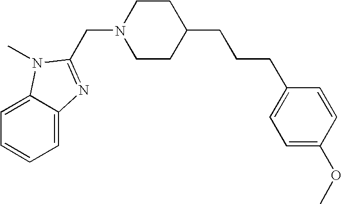 | 2-{4-[3-(4-Methoxy- phenyl)-propyl]- piperidin-1-ylmethyl}- 1-methyl-1H- benzoimidazole |
| 1.17 | 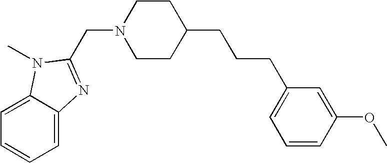 | 2-{4-[3-(3-Methoxy- phenyl)-propyl]- piperidin-1-ylmethyl}- 1-methyl-1H- benzoimidazole |
| 1.18 | 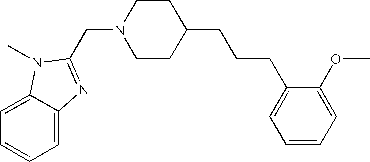 | 2-{4-[3-(2-Methoxy- phenyl)-propyl]- piperidin-1-ylmethyl}- 1-methyl-1H- benzoimidazole |
| 1.19 | 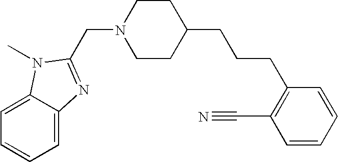 | 2-{3-[1-(1-Methyl-1H- benzoimidazol- 2-ylmethyl)-piperidin-4- yl]-propyl}-benzonitrile |
| 1.20 | 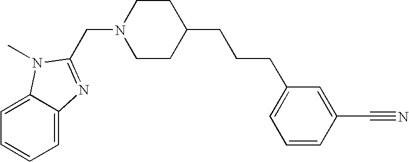 | 3-{3-[1-(1-Methyl-1H- benzoimidazol-2- ylmethyl)-piperidin-4- yl]-propyl}-benzonitrile |
| 1.21 | 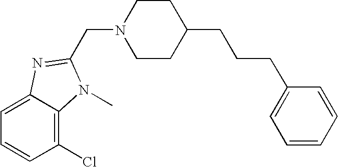 | 7-Chloro-1-methyl-2-[4- (3-phenyl-propyl)- piperidin-1-ylmethyl]- 1H-benzoimidazole |
| 1.22 | 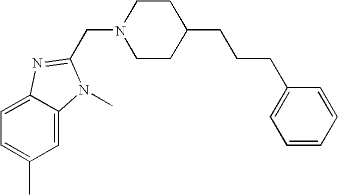 | 1,6-Dimethyl-2-[4-(3- phenyl-propyl)- piperidin-1-ylmethyl]- 1H-benzoimidazole |
| 1.23 | 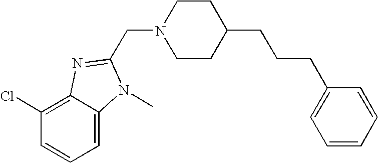 | 4-Chloro-1-methyl-2-[4- (3-phenyl-propyl)- piperidin-1-ylmethyl]- 1H-benzoimidazole |
| 1.24 | 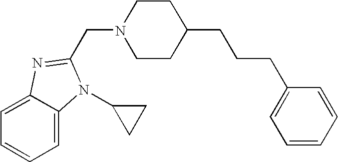 | 1-Cyclopropyl-2-[4-(3- phenyl-propyl)- piperidin-1-ylmethyl]- 1H-benzoimidazole |
| 1.25 | 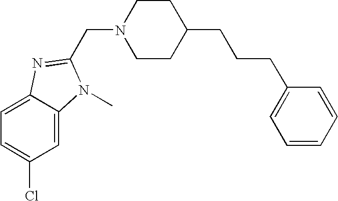 | 6-Chloro-1-methyl-2-[4- (3-phenyl-propyl)- piperidin-1-ylmethyl]- 1H-benzoimidazole |
| 1.26 | 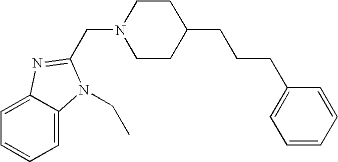 | 1-Ethyl-2-[4-(3-phenyl- propyl)-piperidin-1- ylmethyl]-1H- benzoimidazole |
| 1.27 | 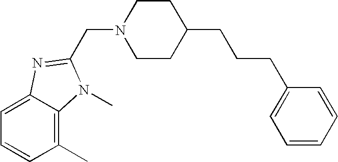 | 1,7-Dimethyl-2-[4-(3- phenyl-propyl)- piperidin-1-ylmethyl]- 1H-benzoimidazole |
| 1.28 | 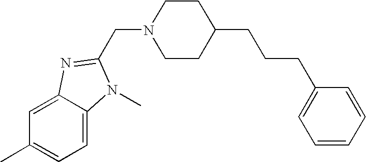 | 1,5-Dimethyl-2-[4-(3- phenyl-propyl)- piperidin-1-ylmethyl]- 1H-benzoimidazole |
| 1.29 | 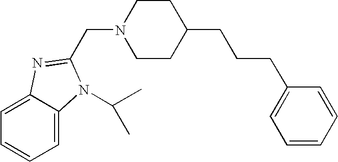 | 1-Isopropyl-2-[4-(3- phenyl-propyl)- piperidin-1-ylmethyl]- 1H-benzoimidazole |
| 1.30 | 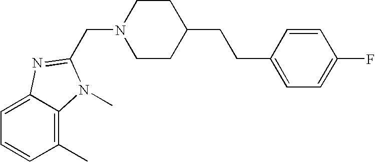 | 2-{4-[2-(4-Fluoro- phenyl)-ethyl]- piperidin-1-ylmethyl}- 1,7-dimethyl-1H- benzoimidazole |
| 1.31 | 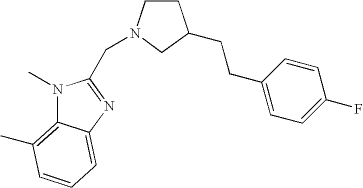 | 2-{3-[2-(4-Fluoro- phenyl)-ethyl]- pyrrolidin-1-ylmethyl}- 1,7-dimethyl-1H- benzoimidazole |
| 1.32 | 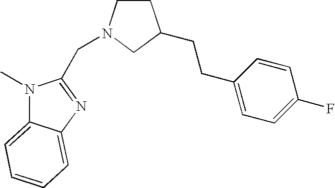 | 2-{3-[2-(4-Fluoro- phenyl)-ethyl]- pyrrolidin-1-ylmethyl}- 1-methyl-1H- benzoimidazole |
| 1.33 | 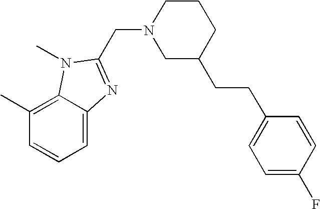 | 2-{3-[2-(4-Fluoro- phenyl)-ethyl]- piperidin-1-ylmethyl}- 1,7-dimethyl-1H- benzoimidazole |
| 1.34 | 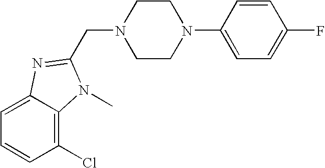 | 7-Chloro-2-[4-(4-fluoro- phenyl)-piperazin-1- ylmethyl]-1-methyl-1H- benzoimidazole |
| 1.35 | 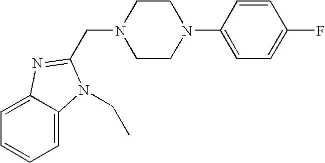 | 1-Ethyl-2-[4-(4-fluoro- phenyl)-piperazin-1- ylmethyl]-1H- benzoimidazole |
| 1.36 | 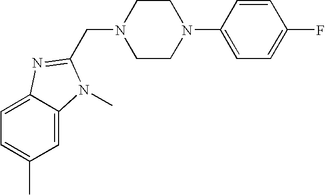 | 2-[4-(4-Fluoro-phenyl)- piperazin-1-ylmethyl]- 1,6-dimethyl-1H- benzoimidazole |
| 1.37 | 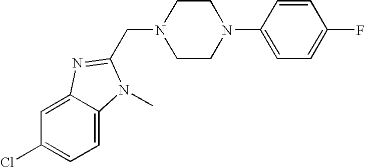 | 5-Chloro-2-[4-(4-fluoro- phenyl)-piperazin-1- ylmethyl]-1-methyl-1H- benzoimidazole |
| 1.38 | 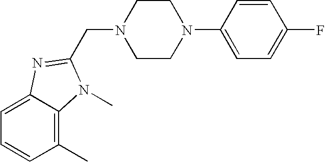 | 2-[4-(4-Fluoro-phenyl)- piperazin-1-ylmethyl]- 1,7-dimethyl-1H- benzoimidazole |
| 1.39 | 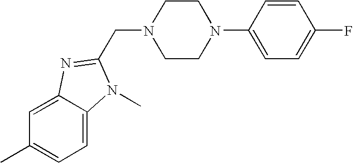 | 2-[4-(4-Fluoro-phenyl)- piperazin-1-ylmethyl]- 1,5-dimethyl-1H- benzoimidazole |
| 1.40 | 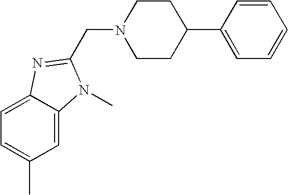 | 1,6-Dimethyl-2-(4- phenyl-piperidin-1- ylmethyl)-1H- benzoimidazole |
| 1.41 | 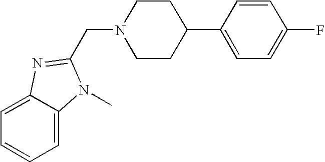 | 2-[4-(4-Fluoro-phenyl)- piperidin-1-ylmethyl]-1- methyl-1H- benzoimidazole |
| 1.42 | 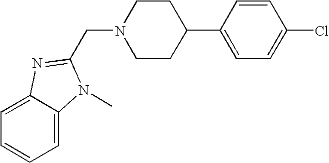 | 2-[4-(4-Chloro-phenyl)- piperidin-1-ylmethyl]-1- methyl-1H- benzoimidazole |
| 1.43 | 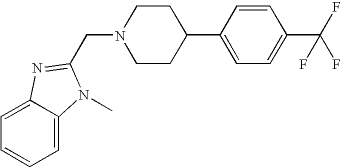 | 1-Methyl-2-[4-(4- trifluoromethyl-phenyl)- piperidin-1-ylmethyl]- 1H-benzoimidazole |
| 1.44 | 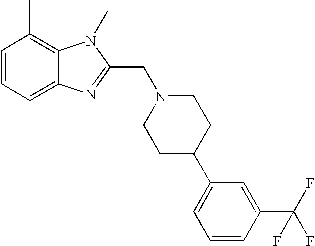 | 1,7-Dimethyl-2-[4-(3- trifluoromethyl-1- phenyl)-piperidin-1- ylmethyl]-1H- benzoimidazole |
| 1.45 | 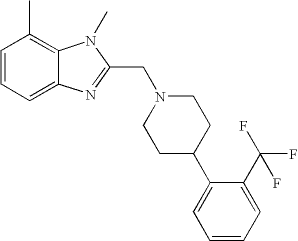 | 1,7-Dimethyl-2-[4-(2- trifluoromethyl-1- phenyl)-piperidin-1-yl methyl]-1H- benzoimidazole |
| 1.46 | 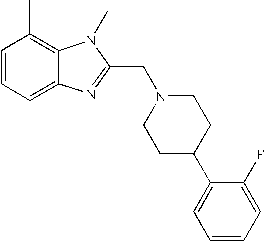 | 2-[4-(2-Fluoro-phenyl)- piperidin-1-ylmethyl]- 1,7-dimethyl-1H- benzoimidazole |
| 1.47 | 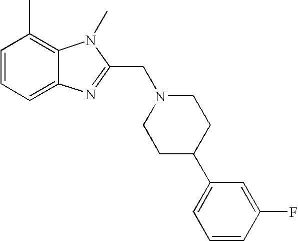 | 2-[4-(3-Fluoro-phenyl)- piperidin-1-ylmethyl]- 1,7-dimethyl-1H- benzoimidazole |
| 1.48 | 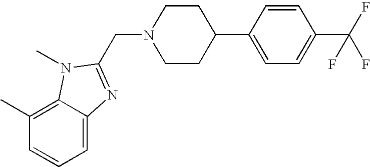 | 1,7-Dimethyl-2-[4-(4- trifluoromethyl-phenyl)- piperidin-1-ylmethyl]- 1H-benzoimidazole |
| 1.49 | 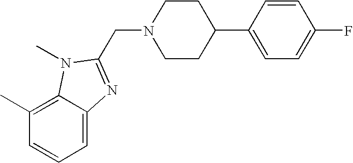 | 2-[4-(4-Fluoro-phenyl)- piperidin-1-ylmethyl]- 1,7-dimethyl-1H- benzoimidazole |
| 1.50 | 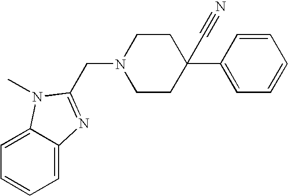 | 1-(1-Methyl-1H- benzoimidazol-2- ylmethyl)-4-phenyl- piperidine-4-carbonitrile |
| 1.51 | 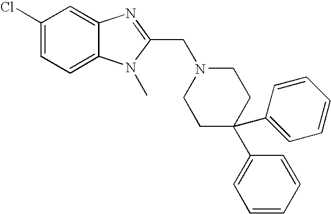 | 5-Chloro-2-(4,4- diphenyl-piperidin-1- ylmethyl)-1-methyl-1H- benzoimidazole |
| 1.52 | 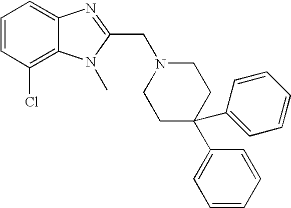 | 7-Chloro-2-(4,4- diphenyl-piperidin-1- ylmethyl)-1-methyl-1H- benzoimidazole |
| 1.53 | 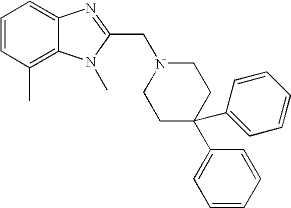 | 2-(4,4-Diphenyl- piperidin-1-ylmethyl)- 1,7-dimethyl-1H- benzoimidazole |
| 1.54 | 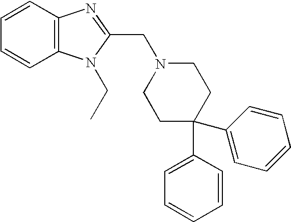 | 2-(4,4-Diphenyl- piperidin-1-ylmethyl)-1- ethyl-1H- benzoimidazale |
| 1.55 | 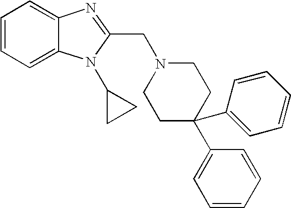 | 1-Cyclopropyl-2-(4,4- diphenyl-piperidin-1- ylmethyl)-1H- benzoimidazole |
| 1.56 | 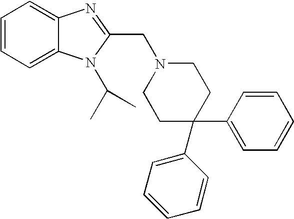 | 2-(4,4-Diphenyl- piperidin-1-ylmethyl)-1- isopropyl-1H- benzoimidazole |
| 1.57 | 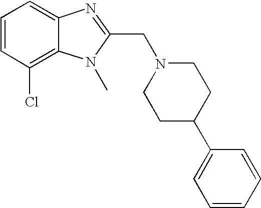 | 7-Chloro-1-methyl-2-(4- phenyl-piperidin-1- ylmethyl)-1H- benzoimidazole |
| 1.58 | 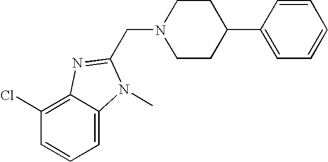 | 4-Chloro-1-methyl-2-(4- phenyl-piperidin-1- ylmethyl)-1H- benzoimidazole |
| 1.59 | 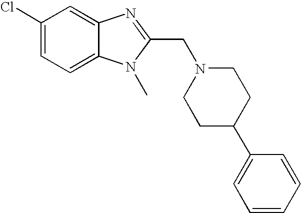 | 5-Chloro-1-methyl-2-(4- phenyl-piperidin-1- ylmethyl)-1H- benzoimidazole |
| 1.60 | 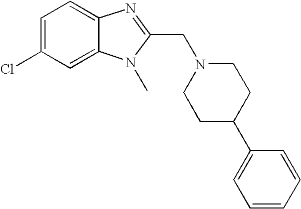 | 6-Chloro-1-methyl-2-(4- phenyl-piperidin-1- ylmethyl)-1H- benzoimidazole |
| 1.61 | 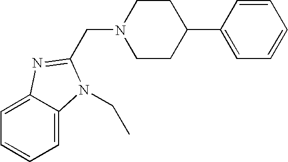 | 1-Ethyl-2-(4-phenyl- piperidin-1-ylmethyl)- 1H-benzoimidazole |
| 1.62 | 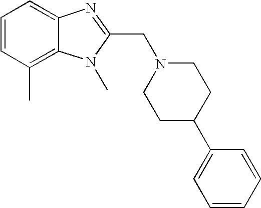 | 1,7-Dimethyl-2-(4- phenyl-piperidin-1- ylmethyl)-1H- benzoimidazole |
| 1.63 | 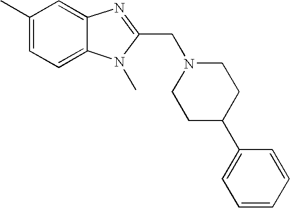 | 1,5-Dimethyl-2-(4- phenyl-piperidin-1- ylmethyl)-1H- benzoimidazole |
| 1.64 | 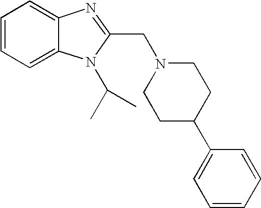 | 1-Isopropyl-2-(4- phenyl-piperidin-1- ylmethyl)-1H- benzoimidazole |
| 1.65 | 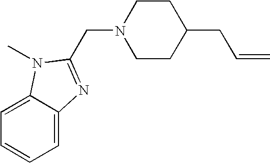 | 2-(4-Allyl-piperidin-1- ylmethyl)-1-methyl-1H- benzoimidazole |
| 1.66 | 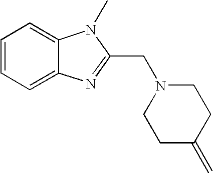 | 1-Methyl-2-(4- methylene-piperidin-1- ylmethyl)-1H- benzoimidazole |
| 1.67 | 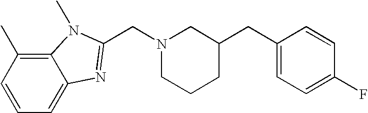 | 2-[3-(4-Fluoro-benzyl)- piperidin-1-ylmethyl]- 1,7-dimethyl-1H- benzoimidazole |
| 1.68 | 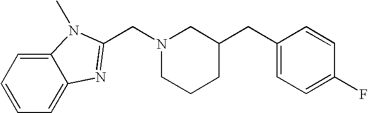 | 2-[3-(4-Fluoro-benzyl)- piperidin-1-ylmethyl]-1- methyl-1H- benzoimidazole |
| 1.69 | 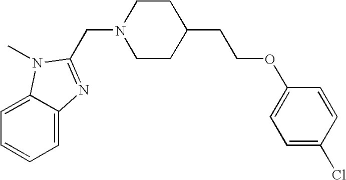 | 2-{4-[2-(4-Chloro- phenoxy)-ethyl]- piperidin-1-ylmethyl}- 1-methyl-1H- benzoimidazole |
| 1.70 | 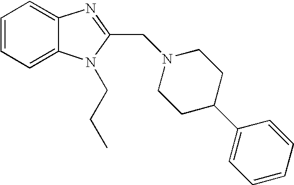 | 2-(4-Phenyl-piperidin-1- ylmethyl)-1-propyl-1H- benzoimidazole |
| 1.71 | 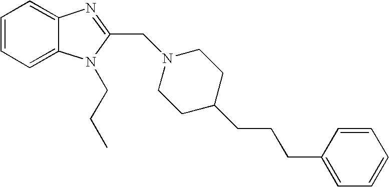 | 2-[4-(3-Phenyl-propyl)- piperidin-1-ylmethyl]-1- propyl-1H- benzoimidazole |
| 1.72 | 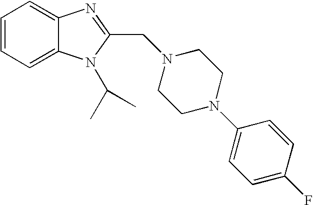 | 2-[4-(4-Fluoro-phenyl)- piperzin-1-ylmethyl]-1- isopropyl-1H- benzoimidazole |
| 1.73 | 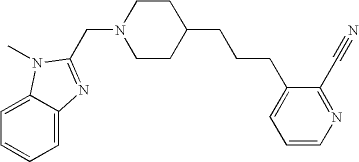 | 3-{3-[1-(1-Methy1-1H- benzoimidazol-2- ylmethyl)-piperidin-4- yl]-propyl}-pyridine-2- carbonitrile |
| 1.74 | 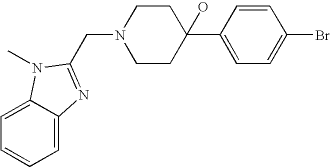 | 4-(4-Bromo-phenyl)-1- (1-methyl-1H- benzoimidazol-2- ylmethyl)-piperidin-4-ol |
| 1.75 | 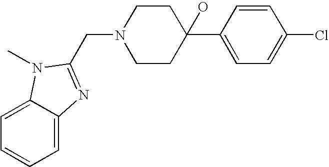 | 4-(4-Chloro-phenyl)-1- (1-methyl-1H- benzoimidazol-2- ylmethyl)-piperidin-4-ol |
| 1.76 | 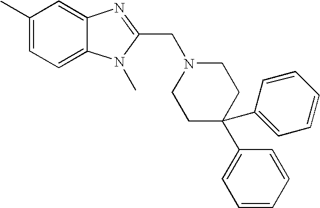 | 2-(4,4-Diphenyl- piperidin-1-ylmethyl)- 1,5-dimethyl-1H- benzoimidazole |
| 1.77 | 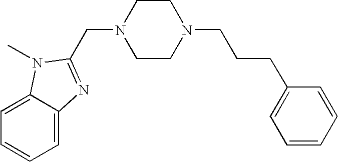 | 1-Methyl-2-[4-(3- phenyl-propyl)- piperazin-1-ylmethyl]- 1H-benzoimidazole |
| 1.78 | 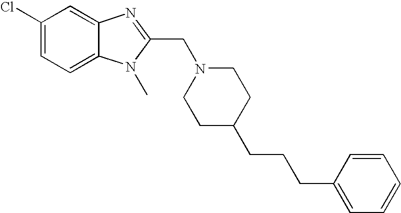 | 5-Chloro-1-methyl-2-[4- (3-phenyl-propyl)- piperidin-1-ylmethyl]- 1H-benzoimidazole |
| 1.79 | 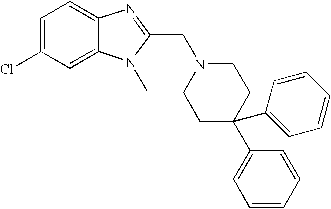 | 6-Chloro-2-(4,4,- diphenyl-piperidin-1- ylmethyl)-1-methyl-1H- benzoimidazole |
| 1.80 | 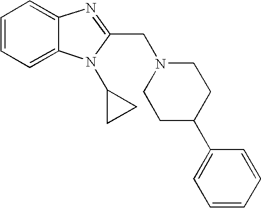 | 1-Cyclopropyl-2-(4- phenyl-piperidin-1- ylmethyl)-1H- benzoimidazole |
| 1.81 | 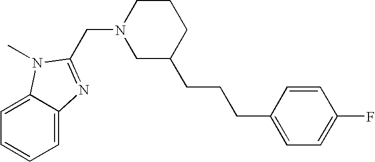 | 2-{3-[3-(4-Fluoro- phenyl)-propyl]- piperidin-1-ylmethyl}- 1-methyl-1H- benzoimidazole |
| 1.82 | 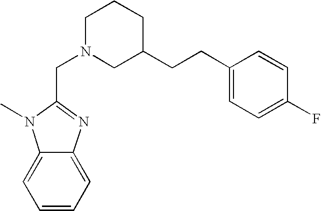 | 2-{3-[2-(4-Fluoro- phenyl)-ethyl]- piperidin-1-ylmethyl}- 1-methyl-1H- benzoimidazole |
| 1.83 | 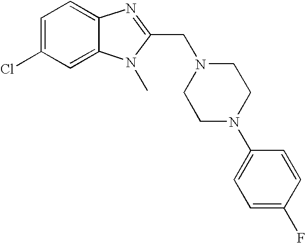 | 6-Chloro-2-[4-(4-fluoro- phenyl)-piperazin-1- ylmethyl]-1-methyl-1H- benzoimidazole |
| 1.84 | 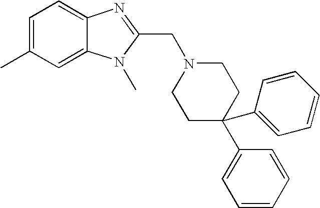 | 2-(4,4-Diphenyl- piperidin-1-ylmethyl)- 1,6-dimethyl-1H- benzoimidazole |
| 1.85 | 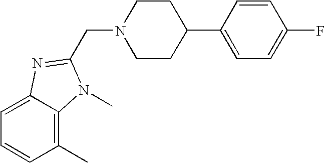 | 2-{3-[4-(4-Fluoro- phenyl)-piperidin- 1-yl]-propyl}-1,7- dimethyl-1H- benzoimidazole |
| 1.86 | 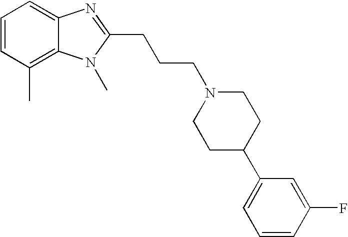 | 2-{3-[4-(3-Fluoro- phenyl)-piperidin- 1-yl]-propyl)-1,7- dimethyl-1H- benzoimidazole |
| 1.87 | 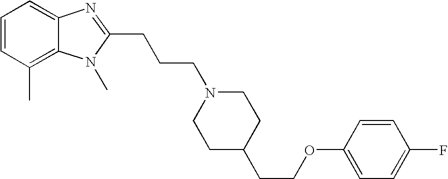 | 2-(3-{4-[2-(4-Fluoro- phenoxy)-ethyl]- piperidin-1-yl}- propyl)-1,7-dimethyl- 1H-benzoimidazole |
| 1.88 | 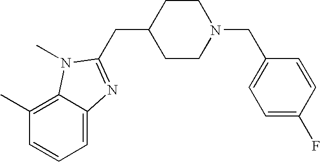 | 2-[1-(4-Fluoro-benzyl)- piperidin-4-ylmethyl]- 1,7-dimethyl-1H- benzoimidazole |
| 2 | 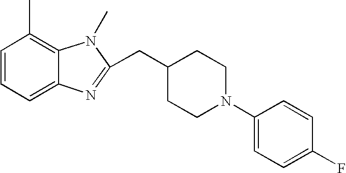 | 2-[1-(4-Fluoro-phenyl)- piperidin-4-ylmethyl]- 1,7-dimethyl-1H- benzoimidazole |
| 22 | 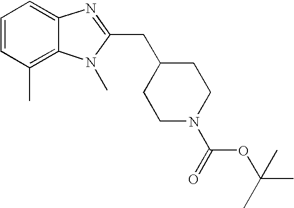 | tert-Butyl 4-[(1-methyl- 7-methyl-1H- benzimidazol-2- yl)methyl]piperidine-1- carboxylate |
| 23 | 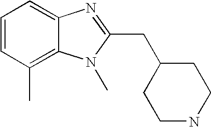 | 1,7-Dimethyl-2- piperidin-4-ylmethyl- 1H-benzoimidazole |
| 26.1 | 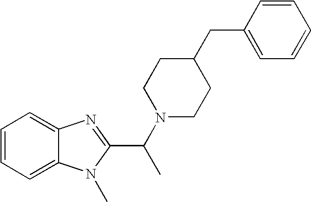 | 2-[1-(4-Benzyl- piperidin-1-yl)-ethyl]-1- methyl-1H- benzoimidazole |
| 26.2 | 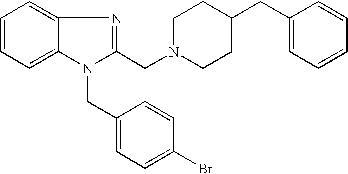 | 2-(4-Benzyl-piperidin- 1-ylmethyl)-1-(4- bromo-benzyl)-1H- benzoimidazole |
| 26.3 | 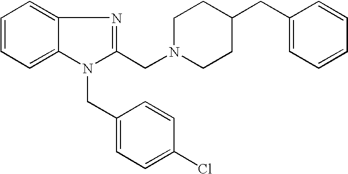 | 2-(4-Benzyl-piperidin- 1-ylmethyl)-1-(4- chloro-benzyl)-1H- benzoimidazole |
| 26.4 | 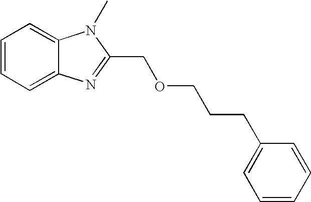 | 1-Methyl-2-(3-phenyl- propoxymethyl)-1H- benzoimidazole |
| 26.5 | 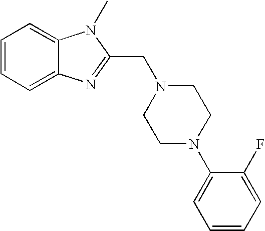 | 2-[4-(2-Fluoro-phenyl)- piperazin-1-ylmethyl]- 1-methyl-1H- benzoimidazole |
| 26.6 | 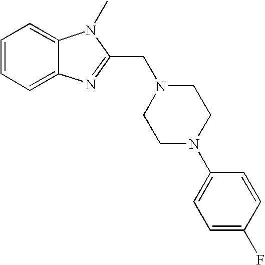 | 2-[4-(4-Fluoro-phenyl)- piperazin-1-ylmethyl]- 1-methyl-1H- benzoimidazole |
| 26.7 | 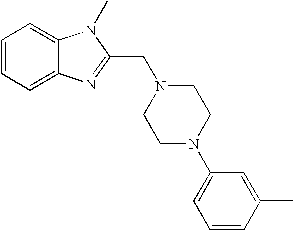 | 1-Methyl-2-(4-m-tolyl- piperazin-1-ylmethyl)- 1H-benzoimidazole |
| 26.8 | 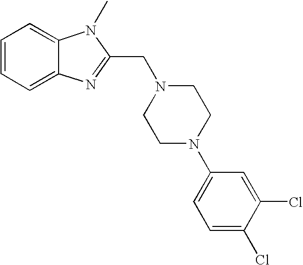 | 2-[4-(3,4-Dichloro- phenyl)-piperazin-1- ylmethyl]-1-methyl-1H- benzoimidazole |
| 26.9 | 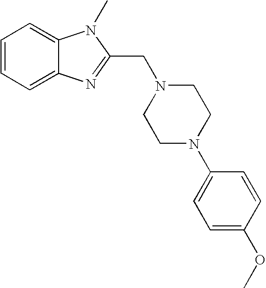 | 2-[4-(4-Methoxy- phenyl)-piperazin-1- ylmethyl]-1-methyl-1H- benzoimidazole |
| 26.10 | 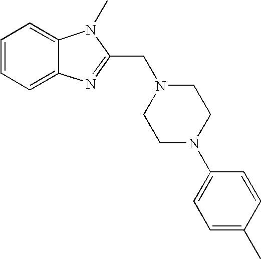 | 1-Methyl-2-(4-p-tolyl- piperazin-1-ylmethyl)- 1H-benzoimidazole |
| 26.11 | 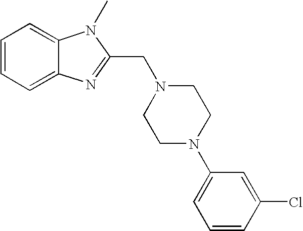 | 2-[4-(3-Chloro-phenyl)- piperazin-1-ylmethyl]- 1-methyl-1H- benzoimidazole |
| 26.12 | 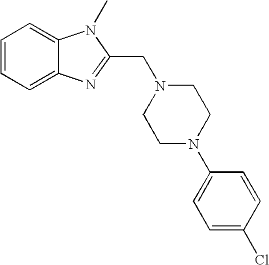 | 2-[4-(4-Chloro-phenyl)- piperazin-1-ylmethyl]- 1-methyl-1H- benzoimidazale |
| 26.13 | 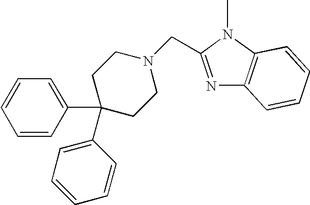 | 2-(4,4-Diphenyl- piperidin-1-ylmethyl)-1- methyl-1H- benzoimidazole |
| 26.14 | 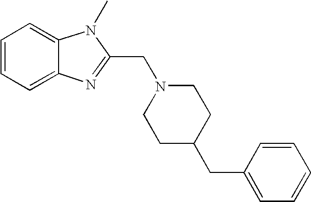 | 2-(4-Benzyl-piperidin- 1-ylmethyl)-1-methyl- 1H-benzoimidazole |
| 26.15 | 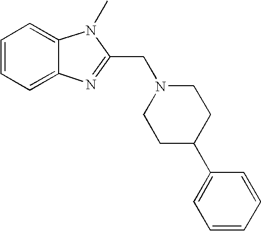 | 1-Methyl-2-(4-phenyl- piperidin-1-ylmethyl)- 1H-benzoimidazole |
| 27 | 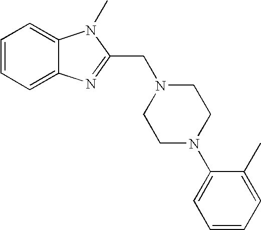 | 1-Methyl-2-(4-o-tolyl- piperazin-1-ylmethyl)- 1H-benzoimidazole |
| 28 | 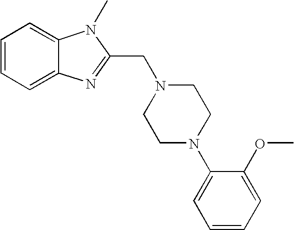 | 2-[4-(2-Methoxy- phenyl)-piperazin-1- ylmethyl]-1-methyl-1H- benzoimidazole |
| 29 | 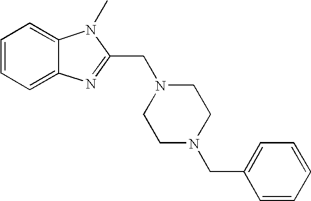 | 2-(4-Benzyl-piperazin- 1-ylmethyl)-1-methyl- 1H-benzoimidazole |
| 30 | 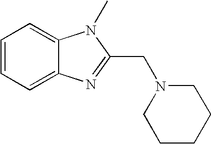 | 1-Methyl-2-piperidin-1- ylmethyl-1H- benzoimidazole |
| 31 | 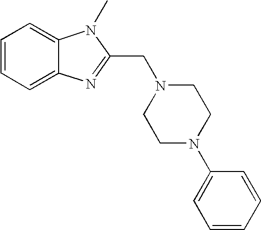 | 1-Methyl-2-(4-phenyl- piperazin-1ylmethyl)- 1H-benzoimidazole |
| 32 | 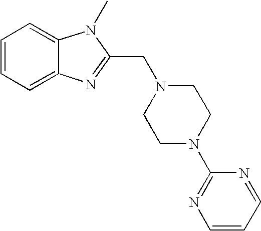 | 1-Methyl-2-(4- pyrimidin-2-yl- piperazin-1-ylmethyl)- 1H-benzoimidazole |
| 33 | 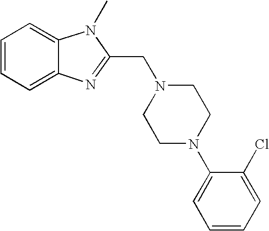 | 2-[4-(2-Chloro-phenyl)- piperazin-1ylmethyl]-1- methyl-1H- benzoimidazole |
| 34 | 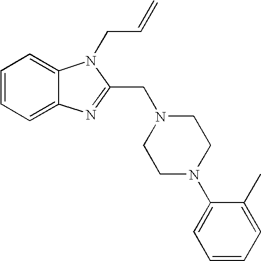 | 1-Allyl-2-(4-o-tolyl- piperazin-1ylmethyl)- 1H-benzoimidazole |
| 35 | 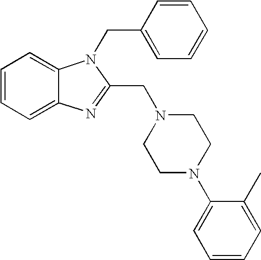 | 1-Benzyl-2-(4-o-tolyl- piperazin-1ylmethyl)- 1H-benzoimidazole |
| 36 | 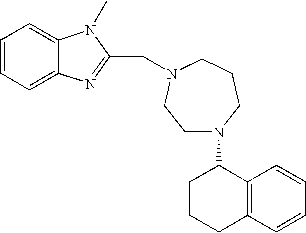 | (S)-1-Methyl-2-[4- (1,2,3,4-tetrahydro- naphthalen-1-yl)- [1,4]diazepan-1- ylmethyl]-1H- benzoimidazole |
| 37 | 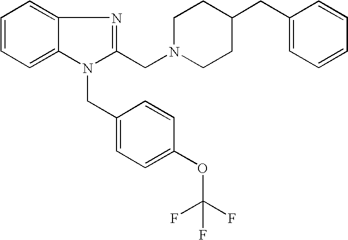 | 2-(4-Benzyl-piperidin- 1-ylmethyl)-1-(4- trifluoromethoxy- benzyl)-1H- benzoimidazole |

### Pharmaceutical Composition

The compounds of the present invention may be formulated into conventional pharmaceutical composition comprising a compound of Formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substance, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

It has been discovered that the compounds of the present invention exhibit activity as pharmaceuticals, in particular as modulators of metabotropic glutamate receptors. More particularly, the compounds of the present invention exhibit activity as potentiators of the mGluR2 receptor, and are useful in therapy, in particular for the treatment of neurological and psychiatric disorders associated with glutamate dysfunction in an animal.

More specifically, the neurological and psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, and conduct disorder.

The invention thus provides a use of any of the compounds according to Formula I, or a pharmaceutically acceptable salt or solvate thereof, for the manufacture of a medicament for the treatment of any of the conditions discussed above.

Additionally, the invention provides a method for the treatment of a subject suffering from any of the conditions discussed above, whereby an effective amount of a compound according to Formula I or a pharmaceutically acceptable salt or solvate thereof, is administered to a patient in need of such treatment. The invention also provides a compound of Formula I or pharmaceutically acceptable salt or solvate thereof, as hereinbefore defined for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses the administration of an effective amount of a compound of the present invention, to mitigate either a preexisting disease state, acute or chronic, or to mitigate a recurring condition. This definition also encompasses prophylactic therapies for prevention of recurring conditions and continued therapy for chronic disorders. In use for therapy in a warm-blooded animal such as a human, the compounds of the present invention may be administered in the form of a conventional pharmaceutical composition by any route including orally, intramuscularly, subcutaneously, topically, intranasally, intraperitoneally, intrathoracically, intravenously, epidurally, intrathecally, intracerebroventricularly and by injection into the joints. In preferred embodiments of the invention, the route of administration is oral, intravenous, or intramuscular.

The dosage will depend on the route of administration, the severity of the disease, age and weight of the patient and other factors normally considered by the attending physician, who determines the individual regimen and dosage level for a particular patient.

As mentioned above, the compounds described herein may be provided or delivered in a form suitable for oral use, for example, in a tablet, lozenge, hard and soft capsule, aqueous solution, oily solution, emulsion, and suspension. Alternatively, the compounds may be formulated into a topical administration, for example, as a cream, ointment, gel, spray, or aqueous solution, oily solution, emulsion or suspension. The compounds described herein also may be provided in a form that is suitable for nasal administration, for example, as a nasal spray, nasal drops, or dry powder. The compounds can be administered to the vagina or rectum in the form of a suppository. The compounds described herein also may be administered parentally, for example, by intravenous, intravesicular, subcutaneous, or intramuscular injection or infusion. The compounds can be administered by insufflation (for example as a finely divided powder). The compounds may also be administered transdermally or sublingually.

In addition to their use in therapeutic medicine, the compounds of Formula I, or salts thereof, are useful as pharmacological tools in the development and standardization of in vitro and in vivo test systems for the evaluation of the effects of inhibitors of mGluR-related activity in laboratory animals as part of the search for new therapeutics agents. Such animals include, for example, cats, dogs, rabbits, monkeys, rats and mice.

### Process for Preparing

Compounds of the present invention can be prepared by various synthetic processes. The selection of a particular process to prepare a given compound is within the purview of the person of skill in the art. The choice of particular structural features and/or substituents may therefore influence the selection of one process over another.

Within these general guidelines, the following processes can be used to prepare exemplary subsets of compounds of this invention. Unless indicated otherwise, the variables described in the following schemes and processes have the same definitions as those given for Formula I, above.

In one process, for example, a compound of Formula I wherein D is methylene, A and B being respectively N and C, may be prepared as shown in Scheme 1, below:

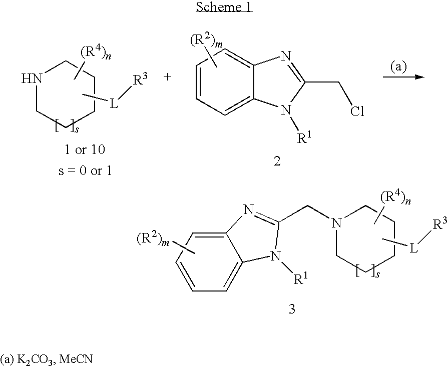

The treatment of 2-chloromethyl-1H-benzimidazole (2) with amine (1 or 10) under basic conditions provides final compound (3).

The above amine (1 or 10) may be prepared as shown in Scheme 2 or 3, below:

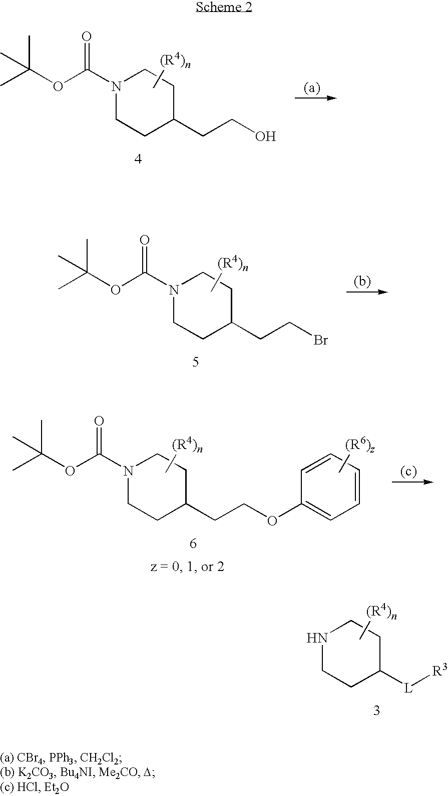

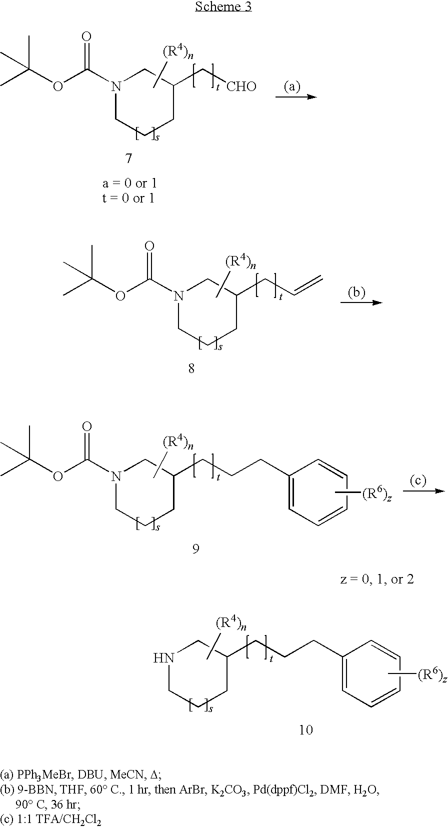

2-chloromethyl-1H-benzimidazole (2) may be prepared as shown in Scheme 4, 5, or 6, below:

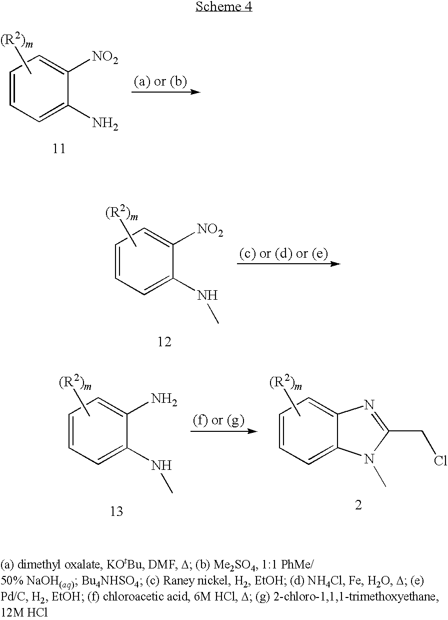

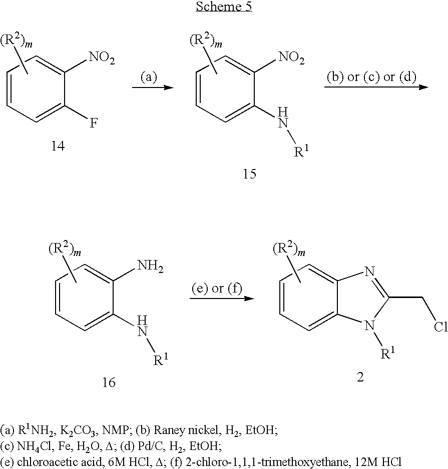

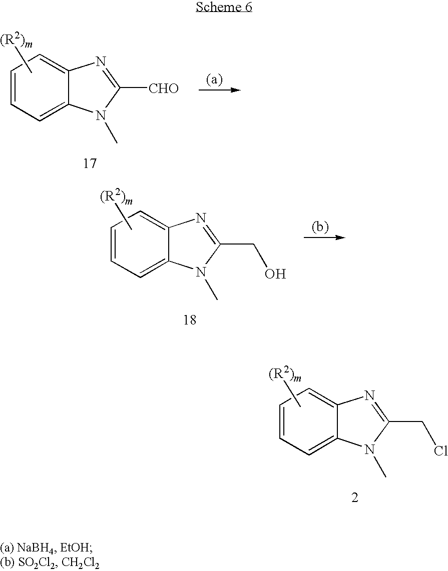

Many variations of the foregoing processes and additions thereto appear throughout the examples that follow. The person of ordinary skill in the art thus will appreciate that compounds of this invention can be prepared by following or adapting one or more of the processes disclosed herein.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### General Methods

All starting materials are commercially available or earlier described in the literature. The ¹H and ¹³C NMR spectra were recorded on a Bruker 300 spectrometer operating at 300 MHz for ¹H NMR, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br or br s: broad singlet, d: doublet, t: triplet, q: quartet, m: multiplet).

Preparative reversed phase chromatography was run on a Gilson autopreparative HPLC with a diode array detector using an XTerra MS C8, 19×300 mm, 7 mm as column.

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns.

Microwave heating was performed in a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in, for example, Aramori et al., 1992, Neuron, 8:757; Tanabe et al., 1992, Neuron, 8:169; Miller et al., 1995, J. Neuroscience, 15:6103; Balazs, et al., 1997, J. Neurochemistry, 1997, 69:151. The methodology described in these publications is incorporated herein by reference. Conveniently, the compounds of the invention can be studied by means of an assay that measures the mobilization of intracellular calcium, [Ca²⁺]ᵢ in cells expressing mGluR2.

A [³⁵S]-GTPγS binding assay was used to functionally assay mGluR2 receptor activation. The allosteric activator activity of compounds at the human mGluR2 receptor was measured using a [³⁵S]-GTPγS binding assay with membranes prepared from CHO cells which stably express the human mGluR2. The assay is based upon the principle that agonists bind to G-protein coupled receptors to stimulate GDP-GTP exchange at the G-protein. Since [³⁵S]-GTPγS is a non-hydrolysable GTP analog, it can be used to provide an index of GDP-GTP exchange and, thus, receptor activation. The GTPγS binding assay therefore provides a quantitative measure of receptor activation.

Membranes were prepared from CHO cells stably transfected with human mGluR2. Membranes (30 μg protein) were incubated with test compound (3 nM to 300 μM) for 15 minutes at room temperature prior to the addition of 1 μM glutamate, and incubated for 30 min at 30° C. in 500 μl assay buffer (20 mM HEPES, 100 mM NaCl, 10 mM MgCl₂), containing 30 μM GDP and 0.1 nM [³⁵S]-GTPγS (1250 Ci/mmol). Reactions were carried out in triplicate in 2 ml polypropylene 96-well plates. Reactions were terminated by vacuum filtration using a Packard 96-well harvester and Unifilter-96, GF/B filter microplates. The filter plates were washed 4×1.5 ml with ice-cold wash buffer (10 nM sodium phosphate buffer, pH 7.4). The filter plates were dried and 35 μl of scintillation fluid (Microscint 20) was added to each well. The amount of radioactivity bound was determined by counting plates on the Packard TopCount. Data was analyzed using GraphPad Prism, and EC₅₀ and Eₘₐₓ values (relative to the maximum glutamate effect) were calculated using non-linear regression.

The following abbreviations are used in the examples:
NMR: nuclear magnetic resonance
HPLC: high performance liquid chromatography
APCI: atmospheric pressure chemical ionization
TMS: tetramethylsilane
CDCl₃: deuterated chloroform
EtOAc: ethyl acetate
DMSO: dimethyl sulfoxide
DCM: dichloromethane
DBU: 8-diazabicyclo[5.4.0]undec-7-ene
9-BBN: 9-borabicyclo[3.3.1]nonane
dppf: 1,1-bis(diphenylphosphanyl)ferrocene
TfOH: trifluoromethanesulfonic acid
DMF: N,N-dimethylformamide
Ty: total yield

Generally, the compounds of the present invention were active in the assays described herein at concentrations (or with EC₅₀ values) of less than 10 μM. Preferred compounds of the invention have EC₅₀ values of less than 1 μM; more preferred compounds of less than about 100 nM. For example, the compounds of Examples 1.2, 1.49, 1.54, 1.75, and 26.8 have EC₅₀ values of 0.057, 0.0795, 1.036, 8.6125, and 0.1865 μM, respectively.

### EXAMPLES

### Example 1.1

### 2-{4-[2-(4-Fluoro-phenoxy)-ethyl]-piperidin-1-ylmethyl}-1-methyl-1H-benzoimidazole

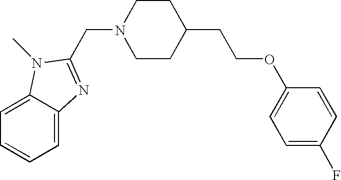

4-[2-(4-Fluoro-phenoxy)-ethyl]-piperidine-1-carboxylic acid tert-butyl ester (68 mg, 0.28 mmol) was dissolved in dichloromethane/trifluoroacetic acid (1:1, 2 mL) for 4 hours. After condensed to dryness, the residue was dissolved in acetonitrile (2 mL), 2-chloromethyl-1-methyl-1H-benzoimidazole (40 mg, 0.18 mmol) and potassium carbonate (124 mg, 0.9 mmol) were mixed together. The reaction mixture was stirred at room temperature for overnight. The reaction mixture was then diluted with ethyl acetate, washed with water and brine, dried over anhydrous sulfate and concentrated in vacuo. The crude residue was purified on silica gel using 2M ammonium in methanol:ethyl acetate=10%:90% to give the product as yellow solid (39.8 mg, 57%).

¹H NMR (300 MHz, CDCl₃): δ 7.73 (d, 1H), 7.3 (m, 3H), 6.97 (t, 2H), 6.82 (dd, 2H), 3.95 (t, 2H), 3.88 (s, 3H), 3.79 (s, 2H), 2.87 (m, 2H), 2.16 (m, 2H), 1.71 (m, 4H), 1.3 (br, 1H), 1.26 (td, 2H)

In a similar fashion the following compounds were synthesized.

| Example | Structure | Name | Yield |
|---|---|---|---|
| 1.2 | 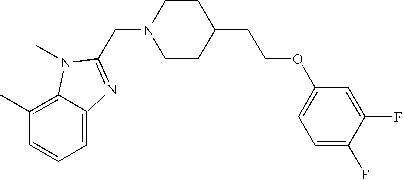 | 2-{4-[2-(3,4-Difluoro- phenoxy)-ethyl]- piperidin-1-ylmethyl}- 1,7-dimethyl-1H- benzoimidazole | 59%, yellow solid |
| NMR | (300 MHz, CDCl3): (ppm) 7.57 (d, 1 H), 7,12 (m, 2 H), 6.98 (t, 1 H), 6.68 (m, 1 H), 6.59 (m, | | |
| | 1 H), 4.12 (s, 3 H), 3.93 (t, 2 H), 3.76 (s, 2 H), 2.87 (m, 2 H), 2.77 (s, 3 H), 2.14 (td, 2 H), 1.7 (m, | | |
| | 4 H), 1.58 (br, 1 H), 1.25 (m, 2 H) | | |
| 1.3 | 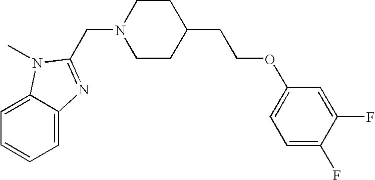 | 2-{4-[2-(3,4-Difluoro- phenoxy)-ethyl]- piperidin-1-ylmethyl}-1- methyl}-1- methyl-1H- benzoimidazole | 68%, brown solid |
| NMR | (300 MHz, CDCl3): (ppm) 7.76 (dd, 1 H), 7.34 (m, 1 H), 7.28 (m, 2 H), 7.03 (q, 1 H), 6.7 (m, | | |
| | 1 H), 6.52 (m, 1 H), 3.96 (td, 2 H), 3.91 (s, 3 H), 3.8 (s, 2 H), 2.98 (m, 2 H), 2.15 (td, 2 H), 1.71 | | |
| | (m, 4 H), 1.51 (br, 1 H), 1.3 (m, 2 H) | | |
| 1.4 | 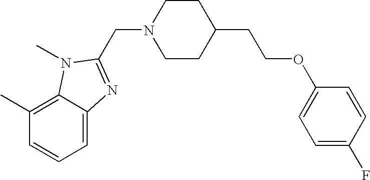 | 2-{4-[2-(4-Fluoro phenoxy)-ethyl]- piperidin-1-ylmethyl}- 1,7-dimethyl-1H- benzoimidazole | 74%, reddish solid |
| NMR | (300 MHz, CDCl3): (ppm) 7.57 (d, 1 H), 7.09 (t, 1 H), 6.98 (m, 3 H), 6.82 (m, 2 H), 4.12 (s, 3 H), | | |
| | 3.96 (t, 2 H), 3.76 (s, 2 H), 2.87 (m, 2 H), 2.76 (s, 3 H), 2.15 (td, 2 H), 1.71 (m, 4 H), 1.55 (br, | | |
| | 1 H), 1.26 m, 2 H) | | |
| 1.5 | 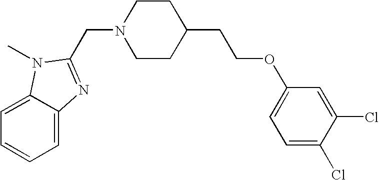 | 2-{4-[2-(3,4-Dichloro- phenoxy)-ethyl]- piperidin-1-ylmethyl)-1- methyl-1H- benzoimidazole | 32%, yellow gum |
| NMR | (300 MHz, CDCl3): (ppm) 7.96 (d, 1 H), 7.78 (m, 2 H), 7.52 (m, 2 H), 6.98 (d, 1 H), 6.76 (dd, | | |
| | 1 H), 4,93 (s, 2 H), 4.19 (s, 3 H), 3.93 (s, 2 H), 3.84 (s, 3 H), 3.8 (s, 2 H), 2.89 (br, 2 H), 2.14 (m, | | |
| | 2 H), 1.72 (m, 2 H) | | |
| 1.6 | 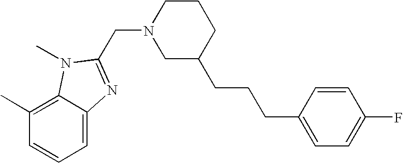 | 2-{3-[3-(4-Fluoro- phenyl)-propyl]- piperidin-1-ylmethyl}- 1,7-dimethyl-1H- benzoimidazole | 66%, brown oil |
| NMR | 7.58 (d, 1 H), 7.08 (m, 3 H), 6.94 (m, 3 H), 4.12 (s, 3 H), 3.72 (s, 2 H), 2.76 (s, 3 H), 2.76 (m, 2 H), | | |
| | 2.54 (t, 2 H), 2.08 (m, 2 H), 1.6 (m, 6 H), 1.22 (m, 2 H), 0.98 (m, 1 H) | | |
| 1.7 | 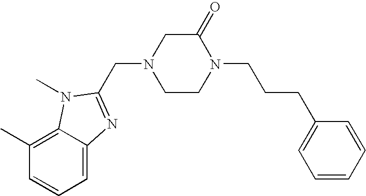 | 4-(1,7-Dimethyl-1H- benzoimidazol-2- ylmethyl)-1-(3-phenyl- propyl)-piperazin-2-one | 41%, reddish oil |
| NMR | (300 MHz, CDCl3): (ppm) 7.59 (d, 1 H), 7.27 (m, 2 H), 7.19 (m, 4 H), 7.14 (d, 1 H), 4.08 (s, | | |
| | 3 H), 3.82 (s, 2 H), 3.45 (t, 2 H), 3.28 (s, 2 H), 3.24 (m, 2 H), 2.77 (s, 3 H), 2.67 (m, 4 H), 1.88 (m, | | |
| | 2 H) | | |
| 1.8 | 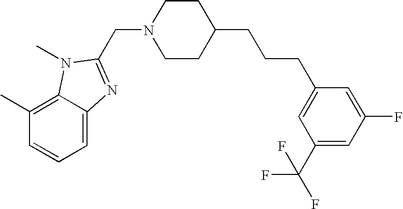 | 2-{4-[3-(3-Fluoro-5- trifluoromethyl-phenyl)- propyl]-piperidin-1- ylmethyl}-1,7-dimethyl- 1H-benzoimidazole | 88%, reddish oil |
| NMR | (300 MHz, CDCl3): (ppm) 7.59 (d, 1 H), 7.23 (s, 1 H), 7.13 (m, 3 H), 6.97 (d, 1 H), 4.11 (s, 3 H), | | |
| | 3.74 (s, 2 H), 2.85 (m, 2 H), 2.76 (s, 3 H), 2.65 (t, 2 H), 2.08 (t, 2 H), 1.28 (m, 4 H), 1.2 (m, 5 H) | | |
| 1.9 | 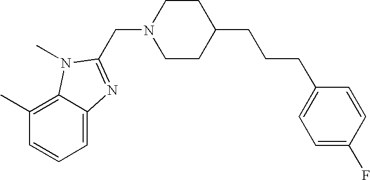 | 2-{4-[3-(4-Fluoro- phenyl)-propyl]- piperidin-1-ylmethyl)- 1,7-dimethyl-1H- benzoimidazole | 69%, reddish oil |
| NMR | (300 MHz, CDCl3) (ppm) 7.59 (d, 1 H), 7.12 (m, 3 H), 6.96 (m, 3 H), 4.11 (s, 3 H), 3.74 (s, | | |
| | 2 H), 2.86 (m, 2 H), 2.82 (s, 3 H), 2.57 (t, 2 H), 2.07 (t, 2 H), 1.62 (m, 4 H), 1.22 (m, 5 H) | | |
| 1.10 | 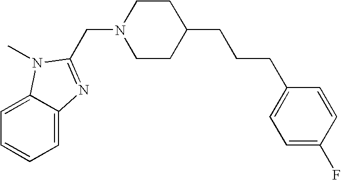 | 2-{4-[3-(4-Fluoro- phenyl)-propyl]- piperidin-1-ylmethyl}-1- methyl-1H- benzoimidazole | 99%, yellow oil |
| NMR | (300 MHz, CDCl3): (ppm) 7.75 (d, 1 H), 7.26 (m, 3 H), 7.12 (m, 2 H), 6.96 (t, 2 H), 3.88 (s, 3 H), | | |
| | 3.78 (s, 2 H), 2.84 (m, 2 H), 2.56 (t, 2 H), 2.1 (t, 2 H), 1,6 (m, 4 H), 1.22 (m, 5 H) | | |
| 1.11 | 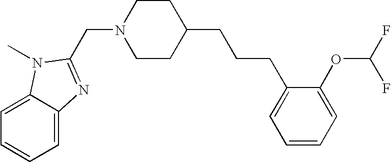 | 2-{4-(3-(2- Difluoromethoxy- phenyl)-propyl]- piperidin-1-ylmethyl}-1- methyl-1H- benzoimidazole | 42%, yellow oil |
| NMR | (300 MHz, CDCl3): (ppm) 7.75 (d, 1 H), 7.26 (m, 3 H), 7.12 (m, 2 H), 6.96 (t, 2 H), 3.88 (s, 3 H), | | |
| | 3.78 (s, 2 H), 2.84 (m, 2 H), 2.56 (t, 2 H), 2.1 (t, 2 H), 1.6 (m, 4 H), 1.22 (m, 5 H) | | |
| 1.12 | 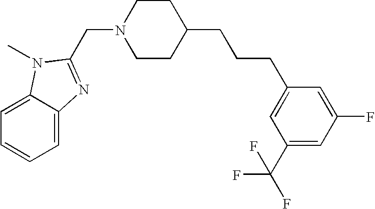 | 2-{4-[3-(3-Fluoro-5- trifluoromethyl-phenyl)- propyl]-piperidin-1- ylmethyl}-1-methyl-1H- benzoimidazole | 69%, yellow oil |
| NMR | (300 MHz, CDCl3): (ppm) 7.75 (d, 1 H), 7.35 (t, 1 H), 7.29 (m, 2 H), 7.23 (s, 1 H), 7.14 (d, 1 H), | | |
| | 7.06 (d, 1 H), 3.88 (s, 3 H), 3.79 (s, 2 H), 2.84 (br, 2 H), 2.65 (t, 2 H), 2.13 (t, 2 H), 1.89 (br, 1 H), | | |
| | 1.65 (m, 4 H), 1.2 (m, 4 H) | | |
| 1.13 | 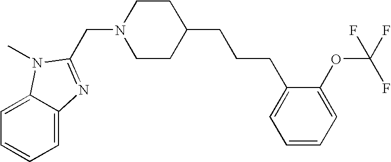 | 1-Methyl-2-{4-[3-(2- trifluoromethoxy phenyl)-propyl]- piperidin-1-ylmethyl}- 1H-benzoimidazole | 15%, colorless oil |
| NMR | (300 MHz, CDCl3): (ppm) 7.76 (d, 1 H), 7.35 (t, 1 H), 7.24 (m, 6 H), 3.89 (s, 3 H), 3.8 (s, 2 H), | | |
| | 2.84 (br, 2 H), 2.65 (t, 1 H), 2.14 (m, 2 H), 1.72 (m, 2 H), 1.66 (m, 4 H), 1.25 (m, 4 H) | | |
| 1.14 | 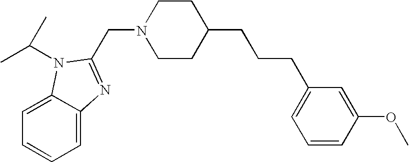 | 1-Isopropyl-2-{4-[3-(3- methoxy-phenyl)- propyl]-piperidin-1- ylmethyl}-1H- benzoimidazole | 75%, yellow oil |
| NMR | (300 MHz, CDCl3): (ppm) 7.74 (m, 1 H), 7.55 (m, 1 H), 7.23 (m, 3 H), 6.75 (m, 3 H), 5.11 (m, | | |
| | 1 H), 3.82 (s, 3 H), 3.77 (s, 2 H), 2.8 (br, 2 H), 2.58 (t, 2 H), 2.11 (t, 2 H), 1.68 (s, 4 H), 1.62 (s, | | |
| | 6 H), 1.27 (m, 3 H), 1.15 (m, 2 H) | | |
| 1.15 | 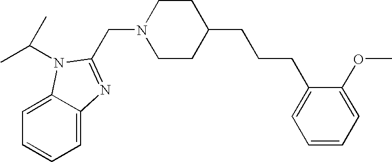 | 1-1sopropyl-2-{4-[3-(2- methoxy-phenyl)- propyl]-piperidin-1- ylmethyl}-1H- benzoimidazole | 47%, yellow oil |
| NMR | (300 MHz, CDCl3): (ppm) 7.77 (m, 1 H), 7.55 (m, 1 H), 7.24 (m, 2 H), 7.18 (m, 2 H), 6.86 (m, | | |
| | 2 H), 5.11 (m, 1 H), 3.82 (s, 3 H), 3.77 (s, 2 H), 2.8 (br, 2 H), 2.59 (t, 2 H), 2.09 (t, 2 H), 1.72 (m, | | |
| | 4 H), 1.63 (s, 6 H), 1.29 (m, 3 H), 1.14 (m, 2 H) | | |
| 1.16 | 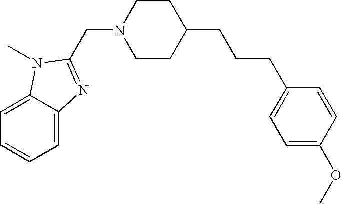 | 2-{4-[3-(4-Methoxy- phenyl)-propyl]- piperidin-1-ylmethyl}-1- methyl-1H- benzoimidazole | 24%, yellow oil |
| NMR | (300 MHz, CDCl3): (ppm) 7.74 (d, 1 H), 7.5 (m, 1 H), 7.3 (m, 2 H), 7.09 (d, 2 H), 6.84 (d, 2 H), | | |
| | 3.89 (s, 3 H), 3.8 (m, 5 H), 2.84 (br, 2 H), 2.53 (t, 2 H), 2.1 (t, 2 H), 1.64 (m, 4 H), 1.25 (m, 5 H) | | |
| 1.17 | 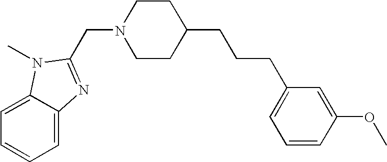 | 2-{4-[3-(3-Methoxy- phenyl)-propyl]- piperidin-1-ylmethyl}-1- methyl-1H- benzoimidazole | 19%, yellow oil |
| NMR | (300 MHz, CDCl3): (ppm) 7.74 (d, 1 H), 7.3 (m, 1 H), 7.25 (m, 3 H), 6.74 (d, 3 H), 3.89 (s, 3 H), | | |
| | 3.8 (m, 5 H), 2.83 (br, 2 H), 2.57 (t, 2 H), 2.1 (t, 2 H), 1.65 (m, 4 H), 1.27 (m, 5 H) | | |
| 1.18 | 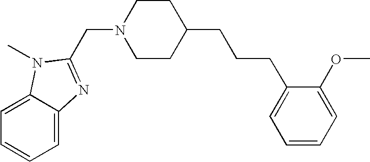 | 2-{4-[3-(2-Methoxy- phenyl)-propyl]- piperidin-1-ylmethyl}-1- methyl-1H- benzoimidazole | 15%, yellow oil |
| NMR | (300 MHz, CDCl3): (ppm) 7.74 (d, 1 H), 7.32 (m, 1 H), 7.27 (m, 2 H), 7.16 (m, 2 H), 6.86 (m, | | |
| | 2 H), 3.9 (s, 3 H), 3.82 (s, 3 H), 3.8 (s, 2 H), 2.87 (br, 2 H), 2.58 (t, 2 H), 2.15 (t, 2 H), 1.66 (m, | | |
| | 4 H), 1.29 (m, 5 H) | | |
| 1.19 | 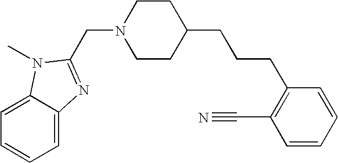 | 2-{3-[1-(1-Methyl-1H- benzoimidazol- 2-ylmethyl)-piperidin-4- yl]-propyl}-benzonitrile | 31%, yellow oil |
| NMR | (300 MHz, CDCl3): (ppm) 7.73 (d, 1 H), 7.59 (d, 1 H), 7.5 (t, 1 H), 7.29 (m, 5 H), 3.88 (s, 3 H), | | |
| | 3.84 (s, 2 H), 2.83 (m, 4 H), 2.12 (m, 3 H), 1.67 (m, 4 H), 1.27 (m, 4 H) | | |
| 1.20 | 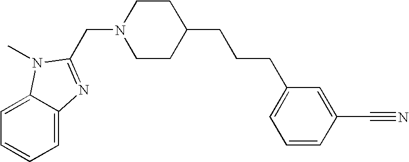 | 3-{3-[1-(1-Methyl-1H- benzoimidazol-2- ylmethyl)-piperidin-4- yl]-propyl)-benzonitrile | 31%, yellow oil |
| NMR | (300 MHz, CDCl3): (ppm) 7.74 (d, 1 H), 7.46 (m, 2 H), 7.37 (m, 3 H), 7.27 (m, 2 H), 3,87 (s, | | |
| | 3 H), 3.7 (s, 2 H), 2.85 (br, 2 H), 2.63 (t, 2 H), 2.14 (t, 2 H), 1,9 (br, 1 H), 1.62 (m, 4 H), 1.19 (m, | | |
| | 4 H) | | |
| 1.21 | 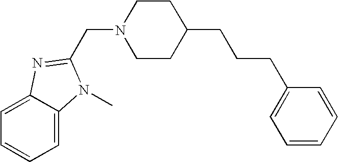 | 7-Chloro-1-methyl-2-[4- (3-phenyl-propyl)- piperidin-1-ylmethyl]- 1H-benzoimidazole | 28%, yellow oil |
| NMR | (300 MHz, CDCl3): (ppm) 1.198-1.448 (m, 6 H, 2(C—CH₂—C); C₂—CH—C), 1.654 (t, 2 H, C—CH₂—C), | | |
| | 1.77 (s, 1 H), 2.145 (t, 2 H, C—CH₂—C), 2.603 (t, 2 H, N—CH₂—C), 2.860 (d, 2 H, N—CH₂—C), | | |
| | 3.752 (s, 2 H, C—CH₂—N), 4.206 (s, 3 H, N—CH₃), 7.10-7.31 (m, 7 H), 7.64 (d, 1 H, H—Ar). | | |
| 1.22 | 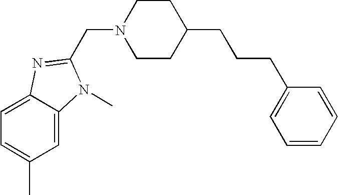 | 1,6-Dimethyl-2-[4-(3- phenyl-propyl)-piperidin- 1-ylmethyl]-1H- benzoimidazole | 98%, yellow gum |
| NMR | (300 MHZ, CDCl3): (ppm) 1.261 (m, 5 H, 2(C—CH₂—C); C₂—CH—C), 1.639 (m, 4 H, 2(C—CH₂—C), | | |
| | 2.096 (t, 2 H, C—CH₂—C), 2.323 (s, 3 H, C—CH₃), 2.527 (t, 2 H, N—CH₂—C), 2.629 (d, 2 H, N—CH₂—C), | | |
| | 3.761 (s, 2 H, ═C—CH₂—N), 3.841 (s, 3 H, N—CH₃), 7.07-7.29 (m, 7 H), 7.63 (d, 1 H). | | |
| 1.23 | 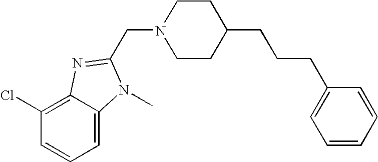 | 4-Chloro-1-methyl-2-[4- (3-phenyl-propyl)- piperidin-1-ylmethyl]- 1H-benzoimidazole | 99%, pale yellow oil |
| NMR | (300 MHz, CDCl3): (ppm) 1.223 (m, 5 H, 2(CH—CH₂—C; C₂—CH—C), 1.640 (t, 4 H, 2(C—CH₂—C)), | | |
| | 2.126 (t, 2 H, C—CH₂—C), 2.601 (t, 2 H, N—CH₂—C), 2.801 (d, 2 H, N—CH₂—C), 3.824 (s, 2 H, | | |
| | ═C—H₂—N), 3.881 (s, 3 H, N—CH₃), 7.160-7.316 (m, 8 H, H—Ar). | | |
| 1.24 | 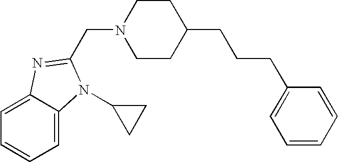 | 1-Cyclopropyl-2-[4-(3- phenyl-propyl)-piperidin- 1-ylmethyl]-1H- benzoimidazole | 77%, yellow gum |
| NMR | (300 MHz, CDCl3): (ppm) 1.196 (m, 4 H, 2(C—CH₂—C)), 1.300 (m, 5 H, 2(C—CH₂—C); C—CH—C₂), | | |
| | 1.646 (t, 4 H, 2-C—CH₂—C), 2.154 (t, 2 H, C—CH₂—C), 2.603 (t, 2 H, N—CH₂—C), 2.939 (d, 2 H, | | |
| | N—CH₂—C), 3.354 (quin, 1 H, N—CH—C₂), 3.831 (s, 2 H, ═C—CH—N), 7.177-7.318 (d of m, 7 H, | | |
| | H—Ar), 7.551 (d, 1 H, H—Ar), 7.764 (d, 1 H, H—Ar). | | |
| 1.25 | 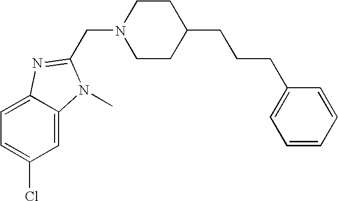 | 6-Chloro-1-methyl-2-[4- (3-phenyl-propyl)- piperidin-1-ylmethyl]- 1H-benzoimidazole | Quantity yield, yellow oil |
| NMR | (300 MHz, CDCl3): (ppm) 1.259 (m, 5 H, 2(C—H₂—C); C₂—CH—C), 1.634 (m, 4 H, 2(C—CH₂—C), | | |
| | 2.133 (t, 2 H, C—CH₂—C), 2.573 (t, 2 H, N—CH₂—C), 2.845 (d, 2 H, N—CH₂—C), 3.759 (s, 2 H, | | |
| | ═C—CH₂—N), 3.845 (s, 3 H, N—CH₃), 7.169-7.336 (m, 7 H, H—Ar), 7.650 (d, 1 H, H—Ar). | | |
| 1.26 | 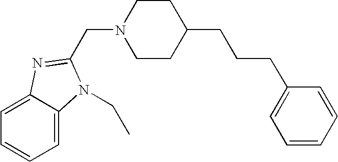 | 1-Ethyl-2-[4-(3-phenyl- propyl)-piperidin-1- ylmethyl]-1H- benzoimidazole | 81%, colorless oil |
| NMR | (300 MHz, CDCl3): (ppm) 1.199 (quint, 1 H, (C₂—CH—C), 1.288 (t, 4 H, 2(C—CH₂—C)), 1.4668 (t, | | |
| | 3 H, C—CH₃), 1.653 (t, 4 H, 2(C—CH₂—C)), 2.114 (t of d, 2 H, C—CH₂—C), 2.610 (t, 2 H, N—CH₂—C), | | |
| | 2.838 (d, 2 H, N—CH₂—C), 3.782 (s, 2 H, ═C—CH₂—N), 4.360 (quart, 2 H, N—CH₂—C), 7.178-7.378 | | |
| | (m, 7 H, H—Ar), 7.758 (m, 1 H, H—Ar). | | |
| 1.27 | 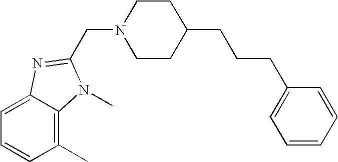 | 1,7-Dimethyl-2-[4-(3- phenyl-propyl)-piperidin- 1-ylmethyl]-1H- benzoimidazole | 8.2%, yellow oil |
| NMR | (300 MHz, CDCl3): (ppm) 1.166 (m, C—CH₂—C; C₂—CH—C), 1.290 (t, 2 H, C—CH₂—C), 1.645 (t, | | |
| | 4 H, 2(C—CH₂—C)), 2.137 (t, 2 H, C—CH₂—C), 2.507 (s, 3 H, C—CH₃), 2.635 (t, 2 H N—CH₂—C), | | |
| | 2.828 (d, 2 H, N—CH₂—C), 3.766 (s, 2 H, ═C—CH₂—N), 3.847 (s, 3 H, N—CH₃), 7.133-7.304 (m, | | |
| | 7 H, H—Ar), 7.553 (s, 1 H, H—Ar). | | |
| 1.28 | 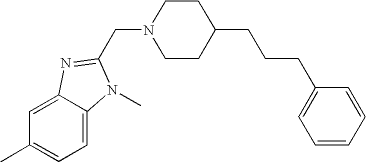 | 1,5-Dimethyl-2-[4-(3- phenyl-propyl)-piperidin- 1-ylmethyl]-1H- benzoimidazole | 78%, yellow oil |
| NMR | (300 MHz, CDCl3): (ppm) 1.166-1.234 (C₂—CH—C; C—CH₂—C), 1.290 (t, 2 H, C—CH₂—C), 1.619 | | |
| | (d, 2 H, C—CH₂—C), 1.693 (d, 2 H, C—CH₂—C), 2.137 (t, 2 H, C—CH₂—C), 2.507 (s, 3 H, C—CH₃), | | |
| | 2.635 (t, N—CH₂—C), 2.867 (d, 2 H, N—CH₂—C), 3.766 (s, 2 H, ═C—CH₂—N), 3.847 (s, 3 H, N—CH₃), | | |
| | 7.133-7.304 (m, 7 H, H—Ar), 7.551 (s, 1 H, H—Ar). | | |
| 1.29 | 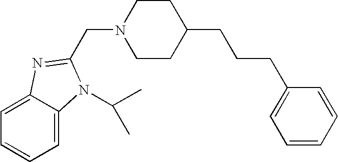 | 1-Isopropyl-2-[4-(3- phenyl-propyl)-piperidin- 1-ylmethyl]-1H- benzoimidazole | 88%, yellow oil |
| NMR | (300 MHz, CDCl3): (ppm) 1.183 (quin, 2 H, 2 H, C—CH₂—C), 1.298 (t, 3 H, C—CH₂—C, C₂—CH—C), | | |
| | 1.642 (d, 6 H, C—(CH₃)₂), 1.666 (d, 2 H, C—CH₂—C), 2.106 (t, 2 H, C—CH₂—C), 2.616 (t, 2 H, | | |
| | N—CH₂—C), 2.790 (d, 2 H, N—CH₂—C), 3.792 (s, 2 H, ═C—CH₂—N), 5.122 (sept, 1 H, N—CH—C₂), | | |
| | 7.184-7.309 (m, 7 H, H—Ar), 7.561-7.789 (d of m, 2 H, H—Ar). | | |
| 1.30 | 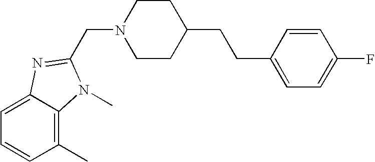 | 2-{4-[2-(4-Fluoro- phenyl)-ethyl]-piperidin- 1-ylmethyl}-1,7- dimethyl-1H- benzoimidazole | 60%, Yellow oil |
| NMR | (300 MHz, CDCl3): (ppm) 7.57 (d, 1 H), 7.13 (m, 3 H), 6.96 (m, 3 H), 4.12 (s, 3 H), 3.75 (s, | | |
| | 2 H), 2.85 (br, 2 H), 2.76 (s, 3 H), 2.59 (m, 2 H), 2.09 (m, 2 H), 1.72 (br, 2 H), 1.52 (m, 2 H), 1.25 | | |
| | (m, 3 H) | | |
| 1.31 | 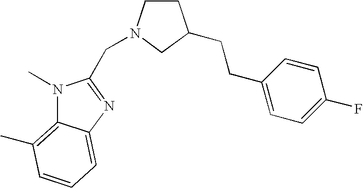 | 2-{3-[2-(4-Fluoro- phenyl)-ethyl]- pyrrolidin-1-ylmethyl}- 1,7-dimethyl-1H- benzoimidazole | 25%, brown oil |
| NMR | (300 MHz, CDCl3): (ppm) 7.59 (d, 1 H), 7.13 (m, 3 H), 6.95 (m, 3 H), 4.14 (s, 3 H), 3.89 (s, | | |
| | 2 H), 2.84 (t, 1 H), 2.76 (s, 3 H), 2.75 (m, 1 H), 2.55 (m, 3 H), 2.12 (m, 3 H), 1.68 (m, 2 H), 1.47 | | |
| | (m, 1 H) | | |
| 1.32 | 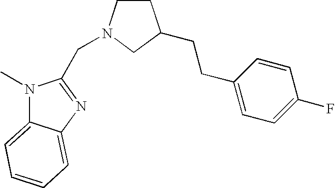 | 2-{3-[2-(4-Fluoro- phenyl)-ethyl]- pyrrolidin-1-ylmethyl)- 1-methyl-1H- benzoimidazole | 70% brown oil |
| NMR | (300 MHz, CDCl3): (ppm) 7.75 (d, 1 H), 7.31 (m, 3 H), 7.09 (dd, 2 H), 6.95 (t, 2 H), 3.92 (s, | | |
| | 2 H), 3.85 (s, 3 H), 2.84 (t, 1 H), 2.7 (m, 1 H), 2.54 (m, 3 H), 2.24 (m, 3 H), 1.67 (q, 2 H), 1.62 (m, | | |
| | 1 H) | | |
| 1.33 | 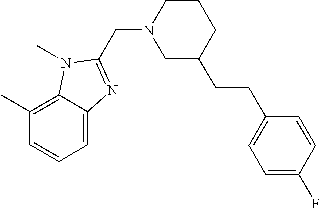 | 2-{3-[2-(4-Fluoro- phenyl)-ethyl]-piperidin- 1-ylmethyl}-1,7- dimethyl-1H- benzoimidazole | 92%, brown oil |
| NMR | (300 MHz, CDCl3): (ppm) 7.6 (d, 1 H), 6.99 (m, 6 H), 4.09 (s, 3 H), 3.75 (s, 2 H), 2.81 (s, 3 H), | | |
| | 2.76 (m, 2 H), 2.54 (m, 2 H), 2.12 (m, 2 H), 1.83 (m, 2 H), 1.66 (m, 1 H), 1.49 (m, 3 H), 1.02 (m, | | |
| | 1 H) | | |
| 1.34 | 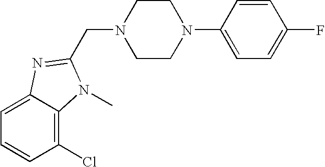 | 7-Chloro-2-[4-(4-fluoro- phenyl)-piperazin-1- ylmethyl]-1-methyl-1H- benzoimidazole | 90%, white solid |
| NMR | (300 MHz, CDCl3): (ppm) 2.720 (t, 4 H, 2(C—CH₂—C)), 3.127 (t, 4 H, 2(N—CH₂—C), 3.872 (s, | | |
| | 2 H, ═C—CH₂—N), 4.233 (s, 3 H, N—CH₃), 6.857-7.005 (m, 4 H, H—Ar), 7.159 (t, 1 H, H—Ar), 7.249 | | |
| | (d of d, 1 H, H—Ar), 7.658 (d of d, 1 H, H—Ar) | | |
| 1.35 | 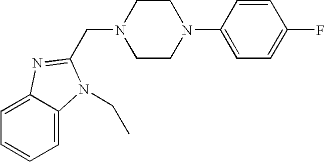 | 1-Ethyl-2-[4-(4-fluoro- phenyl)-piperazin-1- ylmethyl]-1H- beuzoimidazole | white solid 91.3 mg, 90% |
| NMR | (300 MHz, CDCl3): (ppm) 1.490 (t, 3 H, C—CH₃), 2.719 (t, 4 H, 2(N—CH₂—C)), 3.113 (t, 4 H, | | |
| | 2(N—CH₂—C)), 3.885 (s, 2 H, ═C—CH₂—N), 4.374 (quart, 2 H, N—CH₂—C), 6.847-6.996 (m, 4 H, | | |
| | H—Ar), 7.271-7.379 (m, 2 H, H—Ar), 7.382 (m, 1 H, H—Ar), 7.766 (m, 1 H, H—Ar). | | |
| 1.36 | 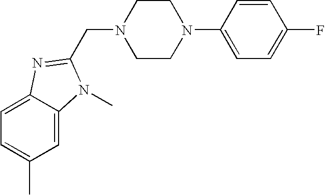 | 2-[4-(4-Fluoro-phenyl)- piperazin-1-ylmethyl]- 1,6-dimethyl-1H- benzoimidazole | 90%, white solid |
| NMR | (300 MHz, CDCl3): (ppm) 2.703 (t, 4 H, 2(C—CH₂—C)), 3.107 (t, 4 H, 2(N—CH₂—C)), 3.867 (s, | | |
| | 5 H, N—CH₃;═C—CH₂—N), 6.848-6.996 (m, 4 H, H—Ar), 7.093-7.166 (m, 2 H, H—Ar), 7.654 (d, 1 H, | | |
| | H—Ar). | | |
| 1.37 | 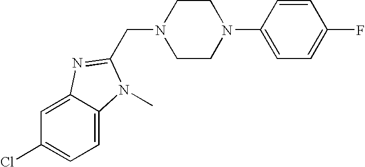 | 5-Chloro-2-[4-(4-fluoro- phenyl)-piperazin-1- ylmethyl]-1-methyl-1H- benzoimidazole | 97%, pale pink solid |
| NMR | (300 MHz, CDCl3): (ppm) 2.696 (t, 4 H, 2(N—CH₂—C)), 3.105 (t, 4 H, 2(N—CH₂—C)), 3.824 (s, | | |
| | 2 H, ═C—CH₂—N), 3.859 (s, 3 H, N—CH₃), 6.833-6.983 (m, 4 H, H—Ar), 7.255 (d, 2 H, H—Ar), | | |
| | 7.723 (s, 1 H, H—Ar). | | |
| 1.38 | 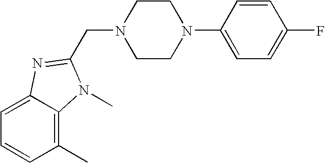 | 2-[4-(4-Fluoro-phenyl)- piperazin-1-ylmethyl]- 1,7-dimethyl-1H- benzoimidazole | 55%, yellow oil |
| NMR | (300 MHz, CDCl3): (ppm) 2.712 (t, 4 H, 2(C—CH₂—C)), 2.776 (s, 3 H, C—CH₃), 3.115 (t, 4 H, | | |
| | 2(N—CH₂—C)), 3.859 (s, 2 H, ═C—CH₂—N), 4.135 (s, 3 H, N—CH₃), 6.853-7.012 (d of m, 5 H, | | |
| | H—Ar), 7.135 (t, 1 H, H—Ar), 7.615 (d, 1 H, H—Ar). | | |
| 1.39 | 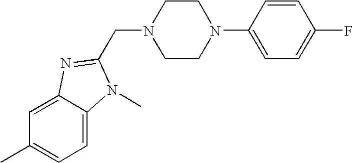 | 2-[4-(4-Fluoro-phenyl)- piperazin-1-ylmethyl]- 1,5-dimethyl-1H- benzoimidazole | 85%, white solid |
| NMR | (300 MHz, CDCl3): (ppm) 2.501 (s, 3 H, C—CH₃), 2.694 (t, 4 H, 2(C—CH₂—C)), 3.097 (t, 4 H, | | |
| | 2(N—CH₂—C), 3.856 (s, N—CH₃, ═C—CH₂—N), 6.834-6.986 (m, 4 H, H—Ar), 7.113-7.252 (m, 2 H, | | |
| | H—Ar), 7.553 (s, 1 H, H—Ar). | | |
| 1.40 | 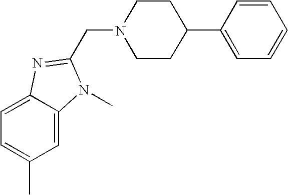 | 1,6-Dimethyl-2-(4- phenyl-piperidin-1- ylmethyl)-1H- benzoimidazole | 96%, white solid |
| NMR | (300 MHz, CDCl3): (ppm) 1.777 (quin of d, 2 H, C—CH₂—C), 1.873 (d, 2 H, C—CH₂—C), 2.244 (t | | |
| | of d, 2 H, N—CH₂—C), 2.539 (t of t, 1 H, C₂—CH—C), 2.553 (s, 3 H, C—CH₃), 2.984 (d, 2 H, | | |
| | N—CH₂—C), 3.841 (s, 2 H, ═C—CH₂—N), 3.892 (s, 3 H, N—CH₃), 7.089-7.637 (m, 7 H, H—Ar), 7.664 (d, 1 H, | | |
| | H—Ar) | | |
| 1.41 | 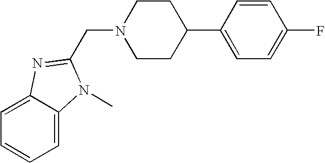 | 2-[4-(4-Fluoro-phenyl)- piperidin-1-ylmethyl]-1- methyl-1H- benzoimidazole | white solid 70.8 mg, 96% |
| NMR | (300 MHz, CDCl3); (ppm) 1.67-1.81 (m, 4 H), 2.23-2.32(td, 2 H), 2.35(dddd, 1 H), 2.98-3.025(d, | | |
| | 2 H), 3.86(s, 3 H), 6.96-7.02(t, 2 H), 6.96-7.02(t, 2 H), 7.15-7.20(m, 2 H), 7.27-7.36(m, 3 H), | | |
| | 7.76-7.83(d, 1 H) | | |
| 1.42 | 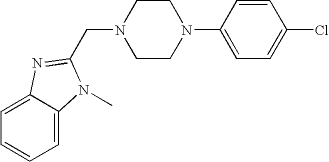 | 2-[4-(4-Chloro-phenyl)- piperidin-1-ylmethyl]-1- methyl-1H- benzoimidazole | yellow solid. 43.5 mg, 102% |
| NMR | (300 MHz, CDCl3): (ppm) 1.67-1.84(m, 4 H), 2.23-2.31 (td, 2 H), 2.52(dddd, 1 H), 2.97-3.03(d, | | |
| | 2 H), 3.861 (s, 2 H), 3.93(s, 3 H), 7.14-7.2(d, 2 H), 7.25-7.37(m, 5 H), 7.76(d, 1 H). | | |
| 1.43 | 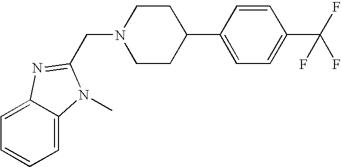 | 1-Methyl-2-[4-(4- trifluoromethyl-phenyl)- piperidin-1-ylmethyl]- 1H-benzoimidazole | yellow brown solid 29.9 mg, 61% |
| NMR | (300 MHz, CDCl3): (ppm) 1.725-1.84(m, 4 H), 2.25-2.62(td, 2 H), 2.623(dddd, 1 H), | | |
| | 3.012-3.05(d, 2 H), 3.87(s, 2 H), 3.94(s, 3 H), 7.27-7.38(m, 5 H), 7.55-7.57(d, 2 H), 7.76(d, 1 H) | | |
| 1.44 | 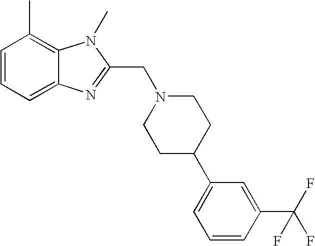 | 1,7-Dimethyl-2-[4-(3- trifluoromethyl-1- phenyl)-piperidin-1- ylmethyl]-1H- benzoimidazole | dark brown gum, 56.72 mg, 71% |
| NMR | 1.72-1.85(m, 4 H). 2.2 (300 MHz, CDCl3): (ppm) 2.28-2.33(dddd, 2 H), 2.61-2.62 (m, 1 H) | | |
| | 2.78(s, 3 H), 3.00-3.04(d, 2 H), | | |
| | 3.83(s, 2 H), 4.15(s, 2 H), 6.98(d, 1 H), 7.10-7.15(t, 1 H), 7.40-7.48(m, 4 H), 7.58-7.60(d, 1 H). | | |
| 1.45 | 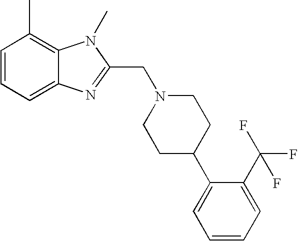 | 1,7-Dimethyl-2-[4-(2- trifluoro methylphenyl)- piperidin-1-yl methyl]- 1H-benzoimidazole | Light brown solid 53 mg, 67% |
| NMR | 1.77-1.82 (m, 3 H). 2.2 (300 MHz, CDCl3): (ppm) 2.26-2.38(m, 2 H), 2.79(s, 3 H) 2.99-3.04(m, | | |
| | 3 H), 3.83(s, 2 H), 4.17(s, 3 H), 6.98-7.01 (d, 1 H), 7.09-7.15(t, 1 H), 7.28-7.29(m, 1 H), | | |
| | 7.49-7.51 (m, 2 H), 7.6-7.6(t, 2 H). | | |
| 1.46 | 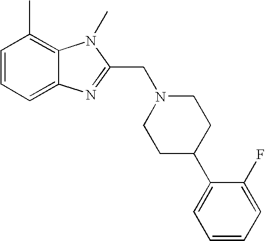 | 2-[4-(2-Fluoro-phenyl)- piperidin-1-ylmethyl]- 1,7-dimethyl-1H- benzoimidazole | yellow brown flaky solid 58 mg, 84% |
| NMR | (300 MHz, CDCl3): (ppm) 1.75-1.88(m, 4 H) 2.28-2.35(m, 2 H), 2.79(s, 3 H), 2.91-3.03(d, 3 H), | | |
| | 3.82(s, 2 H), 4.16(s, 3 H), 6.98-7.28(m, 6 H), 7.58-7.61 (d, 1 H) | | |
| 1.47 | 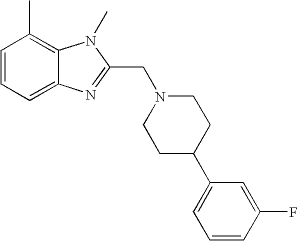 | 2-[4-(3-Fluoro-phenyl)- piperidin-1-ylmethyl]- 1,7-dimethyl-1H- benzoimidazole | yellow powder, 56 mg, 63% |
| NMR | (300 MHz, CDCl3): (ppm) 1.83-1.87(m, 4 H). 2.225-2.233(t, 2 H) 2.56-2.78(m, 1 H), 2.78(s, 3 H), | | |
| | 2.98-3.02(d, 2 H), 3.82(s, 2 H), 4.16(s, 3 H), 6.9-7.02 (m, 4 H), 7.09-7.15 (t, 1 H), 7.25-7.28(q, 1 H), | | |
| | 7.58-7.61(d, 1 H) | | |
| 1.48 | 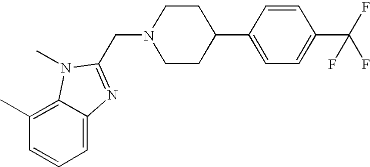 | 1,7-Dimethyl-2-[4-(4- trifluoromethyl-phenyl)- piperidin-1-ylmethyl]- 1H-benzoimidazole | orange brown solid. 55.3 mg, 81% |
| NMR | (300 MHz, CDCl3): (ppm) 1.255-1.302(t, 2 H), 1.72-1.87(m, 4 H), 2.24-2.25(t, 2 H), | | |
| | 2.58-2.62 (m, 2 H), 2.78(s, 3 H), 2.90-3.01(d, 2 H), 3.82(s, 2 H), 4.15(s, 3 H), 6.97-7.00(d, 1 H), | | |
| | 7.15(t, 1 H), 7.324(t, 2 H) 7.55-7.61 (t, 3 H) | | |
| 1.49 | 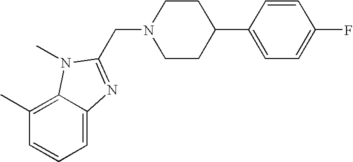 | 2-[4-(4-Fluoro-phenyl)- piperidin-1-ylmethyl]- 1,7-dimethyl-1H- benzoimidazole | clay colored powdery solid 50 mg, 72% |
| NMR | (300 MHz, CDCl3): (ppm) 1.803-1.808(m, 4 H), 2.22-2.23(t, 3 H), 2.26-2.268(m, 1 H), | | |
| | 2.777(s, 3 H), 2.98-3.015(d, 2 H), 3.81(s, 2 H), 4.15(s, 3 H), 6.95-7.02 (m, 3 H), 7.18(d, 1 H), | | |
| | 7.08-7.20(m, 3 H) | | |
| 1.50 | 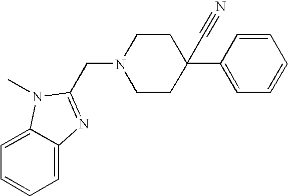 | 1-(1-Methyl-1H- benzoimidazol-2- ylmethyl)-4-phenyl- piperidine-4-carbonitrile. | 51%, yellow solid |
| NMR | (300 MHz, CDCl3): (ppm) 7.77 (d, 1 H), 7.49 (d, 2 H), 7.3 (m, 6 H), 3.93 (s, 2 H), 3.86 (s, 3 H), | | |
| | 3.05 (br, 2 H), 2.73 (td, 2 H), 2.11 (m, 4 H) | | |
| 1.51 | 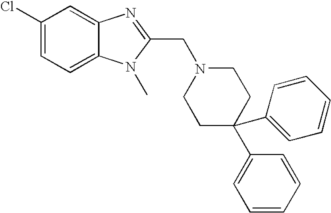 | 5-Chloro-2-(4,4- diphenyl-piperidin-1- ylmethyl)-1-methyl-1H- benzoimidazole | pale pink solid. 63.3 mg, 89% |
| NMR | (300 MHz, CDCl3): (ppm) 2.493 (t, 4 H, 2(C—CH₂—C)), 2.621 (t, 4 H, 2(N—CH₂—C)), 3.715 (s, | | |
| | 2 H, ═C—CH₂—N), 3.862 (s, 3 H, N—CH₃), 7.146-7.718 m, 12 H, H—Ar), 7.722 (s, 1 H, H—Ar) | | |
| 1.52 | 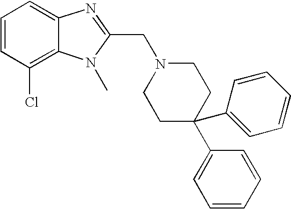 | 7-Chloro-2-(4,4- diphenyl-piperidin-1- ylmethyl)-1-methyl-1H- benzoimidazole | pale pink gum 29.2 mg, 82% |
| NMR | (300 MHz, CDCl3): (ppm) 2.491 (t, 4 H, 2(C—CH₂—C)), 2.625 (t, 4 H, 2(N—CH₂—C)), 3.7 13 (s, | | |
| | 2 H, ═C—CH₂—N), 4.222 (s, 3 H, N—CH₃), 7.136-7.203 (m, 2 H, H—Ar), 7.276-7.324 (m, 10 H, | | |
| | H—Ar), 7.628 (d of d, 1 H, H—Ar). | | |
| 1.53 | 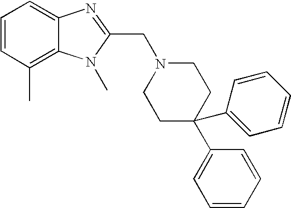 | 2-(4,4-Diphenyl- piperidin-1-ylmethyl)- 1,7-dimethyl-1H- benzoimidazole | white solid 33.2 mg, 70% |
| NMR | (300 MHz, CDCl3): (ppm) 2.416 (t, 4 H, 2(C—CH₂—C)), 2.513 (s, 3 H, C—CH₃), 2.616 (t, 4 H, | | |
| | 2(N—CH₂—C)), 3.728 (s, 2 H, C—CH₂—N), 3.865 (s, 3 H, N—CH₃), 7.146-7.306 (m, 12 H, H—Ar), | | |
| | 7.551 (s, 1 H, H—Ar). | | |
| 1.54 | 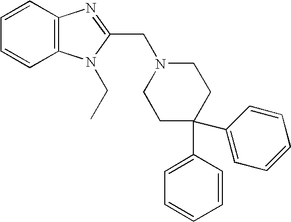 | 2-(4,4-Diphenyl- piperidin-1-ylmethyl)-1- ethyl-1H-benzoimidazole | white solid 121.2 mg, 99% |
| NMR | (300 MHz, CDCl3): (ppm) 1.493 (t, 3 H, C—CH₃), 2.514 (t, 4 H, (2(N—CH₂—C)), 2.644 (t, 4 H, | | |
| | 2(N—CH₂—C)), 3.756 (s, 2 H, ═C—CH₂—H), 4.364 (quart, 2 H, N—CH₂—CH₃), 7.167-7.210 (m, 2 H, | | |
| | H—Ar), 7.282-7.339 (m, 10 H, H—Ar), 7.385-7.797 (m, 1 H, H—Ar), 7.797-7.825 (m, 1 H, H—Ar) | | |
| 1.55 | 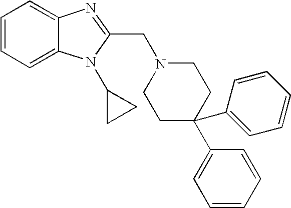 | 1-Cyclopropyl-2-(4,4- diphenyl-piperidin-1- ylmethyl)-1H- benzoimidazole | yellow oil 52.1 mg, 88% |
| NMR | (300 MHz, CDCl3): (ppm) 1.191 (m, 4 H, 2(C—CH₂—C), 2.475 (t, 4 H, 2(C—CH₂—C)), 2.700 (t, | | |
| | 4 H, 2(N—CH₂—C)), 3.351 (quin, 1 H, N—CH—C₂), 3.784 (s, 2 H, ═C—CH₂—N), 7.153 (m, 2 H, | | |
| | H—Ar), 7.284 (10 H, H—Ar), 7.563 (d, 1 H, H—Ar), 7.778 (d, 1 H, H—Ar) | | |
| 1.56 | 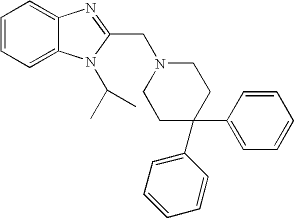 | 2-(4,4-Diphenyl- piperidin-1-ylmethyl)-1- isopropyl-1H- benzoimidazole | yellow oil 95.8 mg, 83% |
| NMR | (300 MHz, CDCl3): (ppm) 1.677 (d, 6 H, C—(CH₃)₂, 2.176 2.484 (br, 4 H, C—CH₂—C), 2.578 (br, | | |
| | 4 H, N—CH₂—C), 3.754(s, 2 H, ═C—CH₂—N), 5.265 (sept, 1 H, N—CH—C₂), 7.146-7.301 (m, 12 H, | | |
| | H—Ar), 7.588 (d of m, 2 H, H—Ar). | | |
| 1.57 | 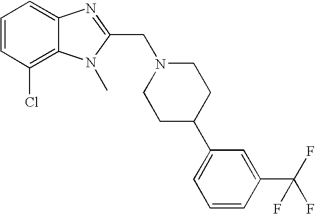 | 7-Chloro-1-methyl-2-(4- phenyl-piperidin-1- ylmethyl)-1H- benzoimidazole | pale yellow gum 22.6 mg, 77% |
| NMR | (300 MHz, CDCl3): (ppm) 1.750 (t of d, 2 H, C—CH₂—C), 1.847 (d, 2 H, C—CH₂—C), 2.255 (t of | | |
| | d, 2 H, N—CH₂—C), 2.557 (t of t, 1 H, C₂—CH—C), 2.985 (d, 2 H, N—CH₂—C), 3.834 (s, 2 H, | | |
| | ═C—CH₂—N), 4.258 (s, 3 H, N—CH₃), 7.121-7.321 (m, 7 H, H—Ar), 7.626-7.656 (d ofd, 1 H, H—Ar) | | |
| 1.58 | 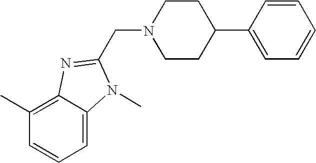 | 4-Chloro-1-methyl-2-(4- phenyl-piperidin-1- ylmethyl)-1H- benzoimidazole | white solid 69.8 mg, 110% |
| NMR | (300 MHz, CDCl3): (ppm) 1.716 (quin of d, 2 H, C—CH₂—C), 1.871 (d, 2 H, C—CH₂—C), 2.302 (t | | |
| | of d, 2 H, N—CH₂—C), 2.998 (d, 2 H, N—CH₂—C), 3.903 (s, 2 H, ═C—CH₂—N), 3.937 (s, 3 H, N—CH₃), | | |
| | 7.206-7.320 (m, 8 H, H—Ar) | | |
| 1.59 | 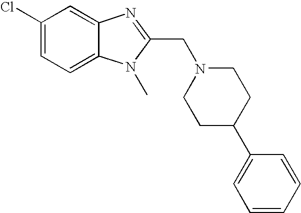 | 5-Chloro-1-methyl-2-(4- phenyl-piperidin-1- ylmethyl)-1H- benzoimidazole | pale pink solid 61.2 mg, 106% |
| NMR | (300 MHz, CDCl3); (ppm) 1.767 (t of d, 2 H, C—CH₂—C), 1.871 (d, 2 H, C—CH₂—C), 2.284 (t of | | |
| | d, 2 H, N—CH₂—C), 2.548 (t of t, 1 H, C₂—CH—C), 3,005 (d, 2 H, N—CH₂—C), 3.830 (s, 2 H, | | |
| | ═C—CH₂—N), 3.912 (s, 3 H, N—CH₃), 7.183-7.316 (m, 7 H, H—Ar), 7.734 (s, 1 H, H—Ar) | | |
| 1.60 | 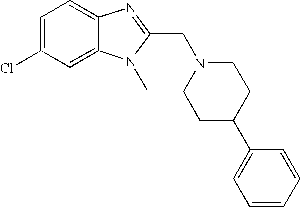 | 6-Chloro-1-methyl-2-(4- phenyl-piperidin-1- ylmethyl)-1H- benzoimidazole | pale yellow solid. 43.2 mg, 98% |
| NMR | (300 MHz, CDCl3): (ppm) 1.736 (quin of d, 2 H, C—CH₂—C), 1.876 (d, 2 H, C—CH₂—C), 2.289 (t | | |
| | of d, 2 H, N—CH₂—C), 2.552 (t of t, 1 H, C₂—CH—C), 3.008 (d, 2 H, N—CH₂—C), 3.841 (s, 2 H, | | |
| | ═C—CH₂—N), 3.899 (s, 3 H, N—CH₃), 7.212-7.362 (m, 7 H, H—Ar), 7.666 (d, 1 H, H—Ar) | | |
| 1.61 | 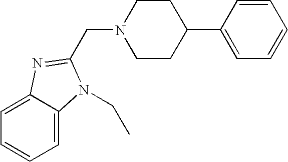 | 1-Ethyl-2-(4-phenyl- piperidin-1-ylmethyl)- 1H-benzoimidazole | pale yellow solid (94.1 mg, 95%). |
| NMR | (300 MHz, CDCl3): (ppm) 1.520 (t, 3 H, C—CH₃), 1.771 (t of d, 2 H, C—CH₂—C), 1.858 (d, 2 H, | | |
| | N—CH₂—C), 2.293 (t of d, 2 H, C—CH₂—C), 2.574 (t of t, 1 H, C₂—CH—C), 3.003 (d, 2 H, N—CH₂—C), | | |
| | 3.863 (s, 2 H, ═C—CH₂—N), 4.382 (quart, 2 H, N—CH₂—C), 7.220-7.396 (m, 8 H, H—Ar), 7.794 (m, | | |
| | 1 H, H—Ar) | | |
| 1.62 | 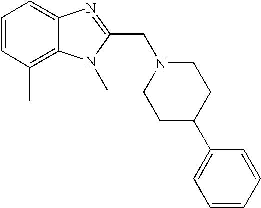 | 1,7-Dimethyl-2-(4- phenyl-piperidin-1- ylmethyl)-1H- benzoimidazole | white solid. (23.1 mg, 40%) |
| NMR | (300 MHz, CDCl3): (ppm) 1.768 (t of d, 2 H, C—CH₂—C), 1.837 (d, 2 H, C—CH₂—C), 2.276 (t of | | |
| | t, 2 H, N—CH₂—C), 2.315 (t of t, 1 H, C₂—CH—C), 2.560 (s, 3 H, C—CH₃), 2.996 (d, 2 H, N—CH₂—C), | | |
| | 3.862 (s, 2 H, ═C—CH₂—N), 4.163 (s, 3 H, N—CH₃), 7.006-7.586 (m, 7 H, H—Ar), 7.590 (d, 1 H, | | |
| | H—Ar) | | |
| 1.63 | 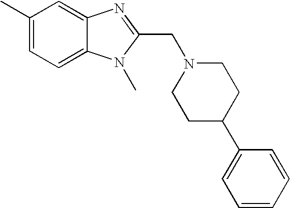 | 1,5-Dimethyl-2-(4- phenyl-piperidin-1- ylmethyl)-1H- benzoimidazole | white solid. 91.3 mg, 92%) |
| NMR | (300 MHz, CDCl3): (ppm) 1.766 (t of d, 2 H, C—CH₂—C), 1.871 (d, 2 H, C—CH₂—C), 2.284 (t of | | |
| | d, 2 H, N—CH₂—C), 2.508 (s, C—CH₃), 2.553 (t of t, 1 H, C₂—CH—C), 2.985 (d, N—CH₂—C), 3.846 | | |
| | (s, 2 H, ═C—CH₂—N), 3.903 (s, 3 H, N—CH₃), 7.151-7.324 (m, 7 H, H—Ar), 7.555 (s, 1 H, H—Ar) | | |
| 1.64 | 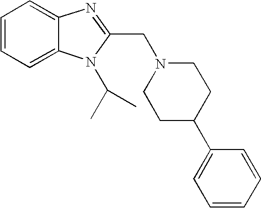 | 1-Isopropyl-2-(4-phenyl- piperidin-1-ylmethyl)- 1H-benzoimidazole | white solid, 84.3 mg, 90% |
| NMR | (300 MHz, CDCl3): (ppm) 1.689 (d, 6 H, C—(CH₃)₂), 1.855 (d of t, 2 H, C—CH₂—C), 1.860 (d, | | |
| | 2 H, C—CH₂—C), 2.284 (t of d, 2 H, N—CH₂—C), 2.576 (t of t, 1 H, C₂—CH—C), 2.988 (d, N—CH₂—C), | | |
| | 3.875 (s, 2 H, C—CH₂—N), 5.167 (sept, 1 H, N—CH—C₂), 7.222-7.334 (m, 7 H, H—Ar), 7.583-7.817 | | |
| | (d of m, 2 H, H—Ar) | | |
| 1.65 | 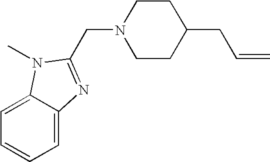 | 2-(4-Allyl-piperidin-1- ylmethyl)-1-methyl-1H- benzoimidazole | 51%, yellow oil |
| NMR | NMR (300 MHz, CDCl3): (ppm) 7.74 (m, 1 H), 7.25 (m, 3 H), 5.8 (m, 1 H), 4.98 (m, 2 H), 3.84 (s, | | |
| | 3 H), 3.81 (s, 2 H), 2.83 (br, 2 H), 2.12 (t, 2 H), 1.97 (t, 2 H), 1.66 (br, 2 H), 1.2 (m, 3 H) | | |
| 1.66 | 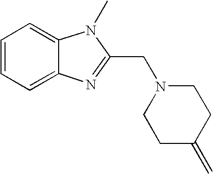 | 1-Methyl-2-(4- methylene-piperidin-1- ylmethyl)-1H- benzoimidazole | 53%, yellow oil |
| NMR | (300 MHz, CDCl3): (ppm) 7.76 (d, 1 H), 7.27 (m, 3 H), 4.68 (s, 2 H), 3.91 (s, 3 H), 3.82 (s, 2 H), | | |
| | 2.54 (t, 4 H), 2.24 (t, 4 H) | | |
| 1.67 | 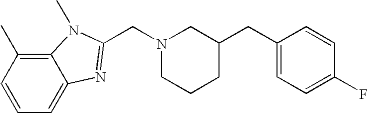 | 2-[3-(4-Fluoro-benzyl)- piperidin-1-ylmethyl]- 1,7-dimethyl-1H- benzoimidazole | 32%, brown solid |
| NMR | (300 MHz, CDCl3): (ppm) 7.58 (d, 1 H), 6.97 (m, 6 H), 4.1 (s, 3 H), 3.75 (s, 2 H), 3.73 (s, 3 H), | | |
| | 2.77 (m, 2 H), 2.74 (m, 1 H), 2.53 (m, 1 H), 2.43 (m, 1 H), 2.18 (m, 1 H), 1,95 (m, 1 H), 1.63 (m, | | |
| | 2 H), 1.45 (m, 1 H), 1.02 (m, 1 H) | | |
| 1.68 | 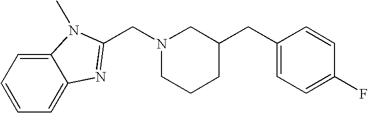 | 2-[3-(4-Fluoro-benzyl)- piperidin-1-ylmethyl]-1- methyl-1H- benzoimidazole | 76%, yellow oil |
| NMR | (300 MHz, CDCl3): (ppm) 7.74 (d, 1 H), 7.35 (m, 1 H), 7.29 (m, 2 H), 7.03 (m, 2 H), 6.9 (t, | | |
| | 2 H), 3.87 (s, 3 H), 3.77 (d, 2 H), 2.74 (br, 2 H), 2.52 (m, 1 H), 2.43 (m, 1 H), 2.12 (br, 1 H), 1.94 | | |
| | (br, 1 H), 1.89 (br, 1 H), 1.66 (br, 2 H), 1.45 (m, 1 H), 1.02 (m, 1 H) | | |
| 1.69 | 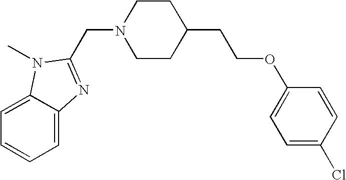 | 2-{4-[2-(4-Chloro- phenoxy)-ethyl]- piperidin-1-ylmethyl}-1- methyl-1H- benzoimidazole | Yellow solid, 2.06 g, 94% |
| NMR | (300 MHz, CDCl3): (ppm) 7.76 (d, 1 H), 7.35 (m, 1 H), 7.26 (m, 2 H), 7.22 (m, 2 H), 6.81 (d, | | |
| | 2 H), 3.96 (t, 2 H), 3.89 (s, 3 H), 3.8 (s, 2 H), 2.88 (br, 2 H), 2.15 (t, 2 H), 1.73 (m, 4 H), 1.67 (m, | | |
| | 1 H), 1.28 (m, 2 H) | | |
| 1.70 | 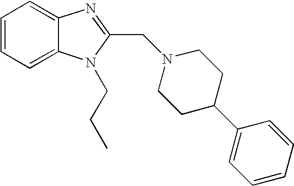 | 2-(4-Phenyl-piperidin-1- ylmethyl)-1-propyl-1H- benzoimidazole | White solid 105.4 mg, 102% |
| NMR | (300 MHz, CDCl3): (ppm) 1.067 (t, 3 H, C—CH₃), 1.785 (t of d, 2 H, C—CH₂—C), 1.859 (d, 2 H, | | |
| | N—CH₂—C), 1.969 (sext, 2 H, C—CH₂—C), 2.298 (t, 2 H, N—CH₂—C), 2.573 (t of t, 1 H, C₂—CH—C), | | |
| | 3.007 (d, 2 H, N—CH₂—C), 3.872 (s, 2 H, ═C—CH₂—N), 4.304 (t, 2 H, N—CH₂—C), 7.223-7.335 (m, | | |
| | 8 H, H—Ar), 7.790 (m, 1 H, H—Ar), | | |
| 1.71 | 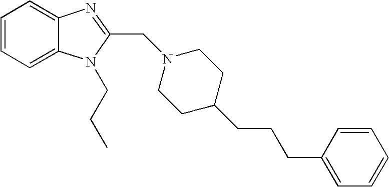 | 2-[4-(3-Phenyl-propyl)- piperidin-1-ylmethyl]-1- propyl-1H- benzoimidazole | Yellow oil 115.1 mg, 99% |
| NMR | (300 MHz, CDCl3): (ppm) 1.022 (t, 3 H, C—CH₃), 1.198 (quin, 1 H, C—CH—C₂), 1.306 (t, 4 H, | | |
| | 2(C—CH₂—C)), 1.637 (t, 4 H, 2(C—CH₂—C)), 1.921 (sext, 2 H, C—CH₂—C), 2.089 (t, 2 H, C—CH₂—C), | | |
| | 2.595 (t, 2 H. C—CH₂—C), 2.850 (d, 2 H, N—CH₂—C), 3.794 (s, 2 H, ═C—CH₂—N), 4.256 (t, 2 H, | | |
| | N—CH₂—C), 7.192-7.310 (m, 8 H, H—Ar), 7,776-7.807 (m, 1 H, H—Ar) | | |
| 1.72 | 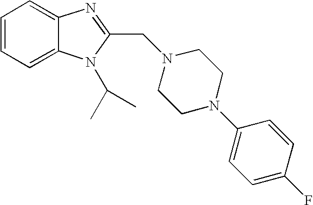 | 2-[4-(4-Fluoro-phenyl)- piperzin-1-ylmethyl]-1- isopropyl-1H- benzoimidazole | white solid 82.3 mg, 83% |
| NMR | (300 MHz, CDCl3): (ppm) 1.675 (d, 6 H, C—(CH₃)₂), 2.682 (t, 4 H, 2(C—CH₂—C)), 3.098 (t, 4 H, | | |
| | 2(—N—CH₂—C)), 3.888 (s, 2 H, ═C—CH₂—N), 5.105 (sext, 1 H, N—CH—(CH₃)₂), 6.840-6.989 (m, 4 H, | | |
| | H—Ar), 7.238-7.282 (m, 2 H, H—Ar), 7.562-7.795 (d of m, 2 H, H—Ar) | | |
| 1.73 | 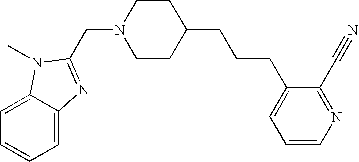 | 3-{3-[1-(1-Methyl-1H- benzoimidazol-2- ylmethyl)-piperidin-4- yl]-propyl}-pyridine-2- carbonitrile | Yellow oil, 78% |
| NMR | (300 MHz, CDCl3): (ppm) 8.56 (d, 1 H), 7.8 (dd, 1 H), 7.66 (dd, 1 H), 7.45 (dd, 1 H), 7.34 (m, | | |
| | 1 H), 7.29 (m, 2 H), 3.88 (s, 3 H), 3.78 (s, 2 H), 2.48 (br, 4 H), 2.12 (br, 2 H), 1.69 (br, 4 H), 1.32 | | |
| | (m, 5 H) | | |
| 1.74 | 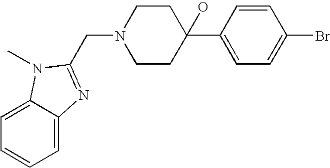 | 4-(4-Bromo-phenyl)-1- (1-methyl-1H- benzoimidazol-2- ylmethyl)-piperidin-4-ol | White solid, 30.3 mg, 86% |
| NMR | (300 MHz, CDCl3): (ppm) 7.62 (d, 1 H), 7.5 (m, 4 H), 7.35 (m, 1 H), 7.29 (m, 1 H), 7.24 (m, | | |
| | 1 H), 3.93 (s, 3 H), 3,91 (s, 2 H), 2.75 (br, 4 H), 2.09 (br, 3 H), 1.78 (br, 2 H) | | |
| 1.75 | 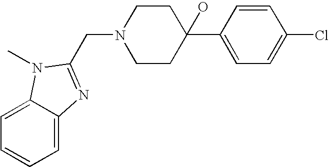 | 4-(4-Chloro-phenyl)-1- (1-methyl-1H- benzoimidazol-2- ylmethyl)-piperidin-4-ol | White solid, 34.6 mg, 110% |
| NMR | (300 MHz, CDCl3): (ppm) 7.62 (d, 1 H), 7.46 (dd, 2 H), 7.34 (m, 2 H), 7.26 (m, 3 H), 3.93 (s, | | |
| | 3 H), 3.91 (s, 2 H), 2.74 (br, 4 H), 2.04 (m, 3 H), 1.79 (br, 2 H) | | |
| 1.76 | 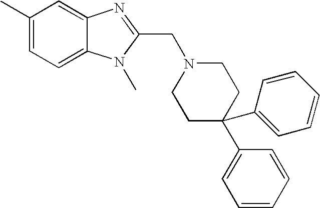 | 2-(4,4-Diphenyl- piperidin-1-ylmethyl)- 1,5-dimethyl-1H- benzoimidazole | white solid. 83.5 mg, 77% |
| NMR | (300 MHz, CDCl3): (ppm) 2.416 (t, 4 H, 2(C—CH₂—C)), 2.616 (C—CH₂—C)), 3.728 (s, 2 H, | | |
| | ═C—CH₂—N), 3.865 (s, 3 H, N—CH₃), 7.146-7.306 (m, 12 H, H—Ar), 7.551 (s, 1 H, H—Ar). | | |
| 1.77 | 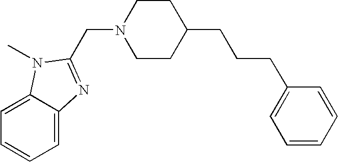 | 1-Methyl-2-[4-(3-phenyl- propyl)-piperazin-1- ylmethyl]-1H- benzoimidazole | Yellow oil, 55 mg, 114% |
| NMR | (300 MHz, CDCl3): (ppm) 7.74 (dd, 1 H), 7.27 (m, 8 H), 3.86 (s, 3 H), 3.82 (s, 2 H), 2.6 (m, | | |
| | 8 H), 2.42 (m, 4 H), 1.84 (m, 2 H) | | |
| 1.78 | 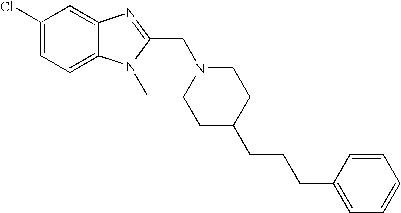 | 5-Chloro-1-methyl-2-[4- (3-phenyl-propyl)- piperidin-1-ylmethyl]- 1H-benzoimidazole | pale pink solid. 60.8 mg, 94% |
| NMR | (300 MHz, CDCl3): (ppm). 1.273 (m, 5 H, 2(C—CH₂—C); C₂—CH—C), 1.654 (m, 4 H, 2(C—CH₂—C), | | |
| | 2.146 (t, 2 H, C—CH₂—C), 2.847 (t, 2 H, N—CH₂—C), 2.885 (d, 2 H, N—CH₂—C), 3.804 (s, 2 H, | | |
| | ═C—CH₂—N), 3.857 (s, 3 H, N—CH₃), 7.164-7.310 (m, 7 H, H—Ar), 7.713 (s, 1 H, H—Ar) | | |
| 1.79 | 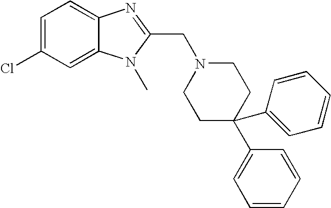 | 6-Chloro-2-(4,4,- diphenyl-piperidin-1- ylmethyl)-1-methyl-1H- benzoimidazole | yellow oil 44.3 mg, 82% |
| NMR | (300 MHz, CDCl3): (ppm). 2.490 (d, 4 H, 2(C—CH₂—C)), 2.620 (d, 4 H, 2(N—CH₂—C)), 3.719 (s, | | |
| | 2 H, ═C—CH₂—N), 3.851 (s, 3 H, N—CH₃), 7.162-7.349 (m, 12 H, H—Ar), 7.652 (d, 1 H, H—Ar) | | |
| 1.80 | 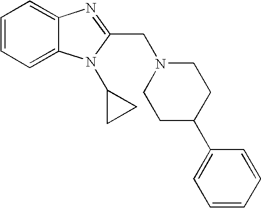 | 1-Cyclopropyl-2-(4- phenyl-piperidin-1- ylmethyl)-1H- benzoimidazole | yellow solid 41.2 mg, 74% |
| NMR | (300 MHz, CDCl3): (ppm). 2.458 (t, 4 H, 2(C—CH₂—C), 2.632 (t, 4 H, 2(N—CH₂—C), 3.726 (s, | | |
| | 2 H, ═C—CH₂—N), 3.855 (s, 3 H, N—CH₃), 7.095-7.307 (m, 12 H, H—Ar), 7.659 (d, 1 H, H—Ar). | | |
| 1.81 | 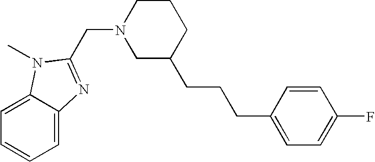 | 2-{3-[3-(4-Fluoro- phenyl)-propyl]- piperidin-1-ylmethyl}-1- methyl-1H- benzoimidazole | Brown oil, 42.4 mg, 74% |
| NMR | (300 MHz, CDCl3): (ppm) 7.76 (dd, 1 H), 7.34 (m, 1 H), 7.27 (m, 2 H), 7.06 (m, 2 H), 6.93 (t, | | |
| | 2 H), 3.88 (s, 3 H), 3.77 (s, 2 H), 2.76 (br, 2 H), 2.52 (t, 2 H), 2.11 (m, 1 H), 1.82 (m, 2 H), 1.52 | | |
| | (m, 5 H), 1.24 (m, 2 H), 0.95 (m, 1 H) | | |
| 1.82 | 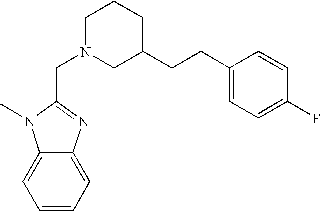 | 2-{3-[2-(4-Fluoro- phenyl)-ethyl]-piperidin- 1-ylmethyl}-1-methyl- 1H-benzoimidazole | Brown oil, 32.1 mg, 91% |
| NMR | (300 MHz, CDCl3): (ppm) 7.75 (dd, 1 H), 7.35 (m, 3 H), 7.03 (m, 2 H), 6.91 (t, 2 H), 3.87 (s, | | |
| | 3 H), 3.78 (s, 2 H), 2.79 (br, 2 H), 2.54 (m, 2 H), 1.97 (m, 1 H), 1.82 (m, 4 H), 1.49 (m, 3 H), 1.04 | | |
| | (m, 1 H) | | |
| 1.83 | 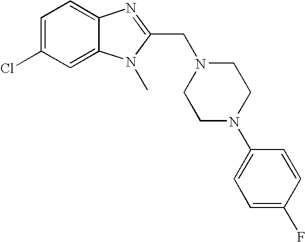 | 6-Chloro-2-[4-(4-fluoro- phenyl)-piperazin-1- ylmethyl]-1-methyl-1H- benzoimidazole | white solid 42.8 mg, 91% |
| NMR | (300 MHz, CDCl3): (ppm). 2.704 (t, 4 H, 2(C—CH₂—C), 3.114 (t, 4 H, 2(N—CH₂—C)), 3.873 (s, | | |
| | 5 H, C—CH₂—N, C—CH₃), 6.846 (m, 4 H, H—Ar), 7.219-7.668 (m, 3 H, H—Ar) | | |
| 1.84 | 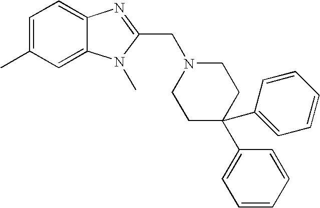 | 2-(4,4-Diphenyl- piperidin-1-ylmethyl)- 1,6-dimethyl-1H- benzoimidazole | white solid 83.2 mg, 91% |
| NMR | (300 MHz, CDCl3): (ppm). 2.458 (t, 4 H, 2(C—CH₂—C), 2.632 (t, 4 H, 2(N—CH₂—C), 3.726 (s, | | |
| | 2 H, ═C—CH₂—N), 3.855 (s, 3 H, N—CH₃), 7.095-7.307 (m, 12 H, H—Ar), 7.659 (d, 1 H, H—Ar). | | |
| 1.85 | 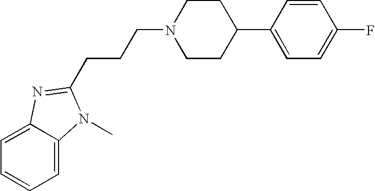 | 2-{3-[4-(4-Fluoro- phenyl)-piperidin- 1-yl]-propyl}-1,7- dimethyl-1H- benzoimidazole | 13 mg |
| NMR | (300 MHz, CDCl3): (ppm) 7.56 (d, 1 H), 7.15 (m, 3 H), 6.96 (m, 3 H), 4.01 (s, 3 H), 3.12 (br, | | |
| | 2 H), 2.95 (t, 2 H), 2.76 (s, 3 H), 2.57 (m, 3 H), 2.13 (m, 4 H), 1.77 (m, 4 H) | | |
| 1.86 | 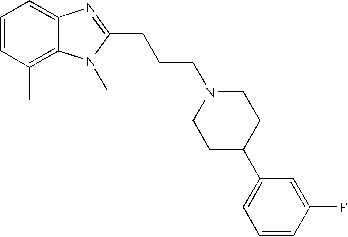 | 2-{3-[4-(3-Fluoro- phenyl)-piperidin- 1-yl]-propyl}-1,7- dimethyl-1H- benzoimidazole | 26 mg |
| NMR | (300 MHz, CDCl3): (ppm) 7.57 (d, 1 H), 7.27 (m, 1 H), 7.1 (t, 1 H), 6.92 (m, 4 H), 4 (s, 3 H), | | |
| | 3.08 (br, 2 H), 2.94 (t, 2 H), 2.76 (s, 3 H), 2.54 (m, 3 H), 2.09 (m, 4 H), 1.79 (m, 4 H) | | |
| 1.87 | 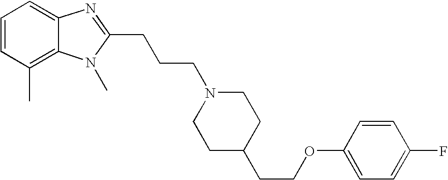 | 2-(3-{4-[2-(4-Fluoro- phenoxy)-ethyl]- piperidin-1-yl}-propyl)- 1,7-dimethyl-1H- benzoimidazole | 23 mg |
| NMR | (300 MHz, CDCl3): (ppm) 7.56 (d, 1 H), 7.1 (t, 1 H), 6.96 (m, 3 H), 6.83 (m, 2 H), 3.97 (m, 5 H), | | |
| | 2.94 (m, 4 H), 2.75 (s, 3 H), 2.53 (t, 2 H), 2.1 (m, 4 H), 1.76 (m, 4 H), 1.55 (br, 1 H), 1.37 (m, 2 H) | | |
| 1.88 | 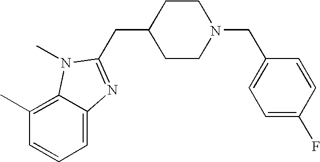 | 2-[1-(4-Fluoro-benzyl)- piperidin-4-ylmethyl]- 1,7-dimethyl-1H- benzoimidazole | White solid, 19 mg, 29% |

| | |
|---|---|
| NMR | (300 MHz, CDCl3): (ppm) 7.56 (d, 1 H), 7.31 (dd, 2 H), 7.1 (t, 1 H), 6.95 (m, 3 H), 3.96 (s, 3 H), |
| | 3.5 (s, 2 H), 2.89 (br, 2 H), 2.82 (d, 2 H), 2.75 (s, 3 H). 2.04 (m, 3 H), 1.78 (m, 2 H), 1.55 (m, 2 H) |

### Example 2

### 2-[1-(4-Fluoro-phenyl)-piperidin-4-ylmethyl]-1,7-dimethyl-1H-benzoimidazole

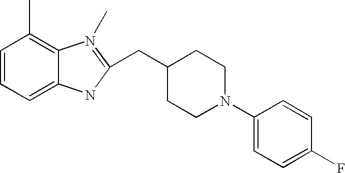

1-Bromo-7-chloro-2-piperidin-4-ylmethyl-1H-benzoimidazole (65 mg, 0.267 mmol), palladium acetate (6 mg, 0.0267 mmol), Cs₂CO3 (260 mg, 0.801 mmol), biphenyl-2-yl-dicyclohexyl-phosphane (9.4 mg, 0.0267 mmol) and 1-fluoro-4-iodo-benzene (71.1 mg, 0.32 mmol) were mixed in toluene (2 mL), the reaction mixture was heated at 100° C. for overnight. The reaction mixture was diluted with dichloromethane and washed with water and brine. The organic phase was separated, dried over anhydrous sodium sulfate, filtered and concentrated in vacuo. The crude residue was purified on silica gel using ethyl acetate:hexine=80%:20%, then methanol:ethyl acetate=4%:96% to give the product as yellow solid (40.1 mg, 44.5%).

¹HNMR (300 MHz, CDCl₃): (ppm) 7.59 (d, 1H), 7.12 (t, 1H), 6.96 (m, 5H), 3.99 (s, 3H), 3.55 (br, 2H), 2.86 (d, 2H), 2.76 (s, 3H), 2.68 (td, 2H), 2.06 (br, 1H), 1.89 (br, 2H), 1.6 (td, 2H)

### Example 3.1

### Methyl-(2-methyl-6-nitro-phenyl)-amine

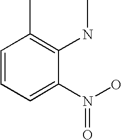

2-Methyl-6-nitro-phenylamine (5.0 g, 32.9 mmol), dimethyl oxalate (5.82 g, 49.3 mmol) and potassium tert-butoxide (5.52 g, 49.3 mmol) were dissolved in N,N-dimethylformamide (50 mL). The reaction mixture was kept refluxing for overnight. The reaction was cooled to room temperature; ethyl acetate was then added to the reaction mixture. The reaction mixture was washed with water and brine. The organic phase was separated, dried over anhydrous sodium sulfate, filtered and concentrated in vacuo. The crude residue was purified on silica gel using ethyl acetate:hexane=20%:80% to give product as yellow solid (2.3 g, 42.1%).

¹H-NMR (300 MHz, CDCl₃): (ppm) 2.413 (s, 3H, C—CH₃), 3.018 (d, 3H, N—CH₃), 7.112 (t, 1H, H—Ar), 7.255 (d, 1H, H—Ar), 7.882 (d, 1H, H—Ar).

In a similar fashion the following compounds were synthesized.

| Example | Structure | Name | Yield |
|---|---|---|---|
| 3.2 | 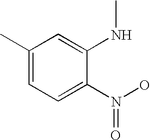 | Methyl-(5-methyl-2-nitro-phenyl)-amine | yellow solid (16.7%) |
| NMR | 2.379 (s, 3 H, C—CH₃), 3.023 (d, 3 H, N—CH₃), 6.466-6.628 (d of t, 1 H, | | |
| | H—Ar), 6.628 (s, 1 H, H—Ar), 8.066 (s, 1 H, H—Ar) | | |
| 3.3 | 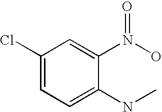 | (4-Chloro-2-nitro-phenyl)-methyl-amine | orange solid (60.0%) |
| NMR | 3.029 (d, 3 H, N—CH₃), 6.802 (d, 1 H, H—Ar), 7.391-7.432 (d of quart, 1 H, | | |
| | H—Ar), 8.028 (br, 1 H, NH), 8.163 (s, 1 H, H—Ar) | | |
| 3.4 | 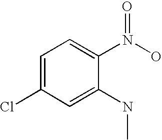 | (5-Chloro-2-nitro-phenyl)-methyl-amine | yellow solid (60.3%) |

| | |
|---|---|
| NMR | 3.00 (d, 3 H, N—CH₃), 6.57 (d, 1 H, H—Ar), 6.61 (s, 1 H, H—Ar), 8.07 (d, |
| | 1 H, H—Ar), 8.10 (br, 1 H, NH) |

### Example 4.1

### (3-Chloro-2-nitro-phenyl)-methyl-amine

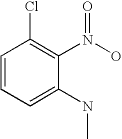

3-Chloro-2-nitro-phenylamine phenylamine (5.0 g, 31.66 mmol) and dimethyl sulfate (4.39 g, 34.82 mmol) were dissolved in 20 mL of 50/50 toluene and concentrated sodium hydroxide. To the reaction tetrabutylammonium hydrogen sulfate (0.643 g, 1.89 mmol) was added, and the reaction was allowed to stir for 6 hours. The reaction mixture was poured into 5% HCl aqueous solution, and extracted with dichloromethane (5×). The organic phase was dried over anhydrous sodium sulfate and concentrated in vacuo. The crude residue was purified on silica gel using ethyl acetate:hexane=20%:L 80% to give product as yellow solid (3.2 g, 53.6%).

¹H-NMR (300 MHz, CDCl₃): (ppm) 2.891 (d, 3H, N—CH₃), 5.943 (br, 1H, NH), 6.701 (t, 2H, H—Ar), 7.245 (t, 1H, H—Ar).

In a similar fashion the following compounds were synthesized.

| Example | Structure | Name | Yield |
|---|---|---|---|
| 4.2 | 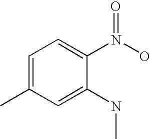 | Methyl-(5-methyl-2-nitro-phenyl)-amine | yellow solid (65.0%) |
| NMR | 2.341 (s, 3 H, C—CH₃), 2.983 d, 3 H, N—CH₃), 6.420 (d, 1 H, H—Ar), 6.588 | | |
| | (d, 1 H, H—Ar), 8.002 (d, 1 H, H—Ar), 8.101 (br, 1 H, NH) | | |
| 4.3 | 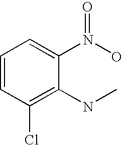 | (2-Chloro-6-nitro-phenyl)-methyl-amine | yellow/orange oil (88.3%) |

| | |
|---|---|
| NMR | 3.099 (d, 3 H, N—CH₃), 6.679 (t, 1 H, H—Ar), 6.734 (br, 1 H, NH), 7.469 (d |
| | of d, 1 H, H—Ar), 7.916 (d of d, 1 H, H—Ar) |

### Example 5

### Cyclopropyl-(2-nitro-phenyl)-amine

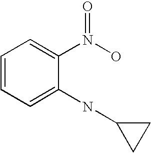

1-Fluoro-2-nitro-benzene (2.8 g, 19.8 mmol), cyclopropyl amine (3 ml), triethylamine (3 mL) were dissolved in acetonitrile (6 mL). The reaction mixture was sealed tightly in a pressure flask and allowed to stir at 110° C. overnight. The reaction mixture was then cooled to room temperature, diluted with ethyl acetate; water was then added. The organic phase was washed with water (3×), dried over anhydrous sodium sulfate and concentrate in vacuo. The crude residue was purified on silica gel using ethyl acetate:hexane=5%:95% to give orange oil (3.62 g, 103.4%).

¹H-NMR (300 MHz, CDCl₃): (ppm) 0.661 (pent, 2H, C—CH₂—C), 0.922 (pent, 2H, C—CH₂—C), 2.574 (sept, 1H, N—CH—C₂), 6.652-6.708 (m, 1H, H—Ar), 7.295-7.466 (d of m, 2H, H—Ar), 8.116-8.145 (m, 1H, H—Ar).

### Example 6.1

### Ethyl-(2-nitro-phenyl)-amine

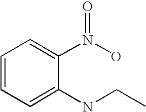

2-Fluoronitrobenzene (1 g, 7.09 mmol) and ethylamine (7.1 mL, 2M in tetrahydrofuran, 14.2 mmol) were added to the suspension of potassium carbonate (1.96 g, 14.2 mmol) in anhydrous N-methylpyrrolidinone (20 mL). The reaction mixture was stirred at room temperature for 3.5 hours, and then poured into water. The mixture was extracted with ethyl acetate. The organic phase was washed with brine, dried over magnesium sulfate, filtered and concentrated in vacuo to give orange oil. The ¹H-NMR showed that this crude product was the desired product; it was used in the later steps without further purification.

¹H-NMR (300 MHz, CDCl₃): (ppm) 8.19 (d, 1H), 7.95 (br, 1H), 7.45 (t, 1H), 6.84 (d, 1H), 6.64 (t, 1H), 3.38 (m, 2H), 2.01 (t, 3H).

In a similar fashion the following compounds were synthesized.

| Example | Structure | Name | Yield |
|---|---|---|---|
| 6.2 | 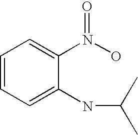 | Isopropyl-(2-nitro-phenyl)-amine | Crude product was used in the later steps without further purification. |

### Example 7.1

### 3,N-2-Dimethyl-benzene-1,2-diamine

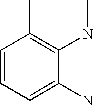

Three scoops of Raney-nickel catalyst were washed with ethanol twice. Methyl-(2-methyl-6-nitro-phenyl)-amine (0.69 mg, 4.18 mmol) was dissolved in 20 mL ethanol, and the solution was added to the Raney-nickel catalyst. The reaction flask was affixed with a balloon filled with hydrogen. The reaction was allowed to stir for 16 hours. The reaction was filtered through a pad of diatomaceous earth and into a flask containing concentrated hydrochloric acid. The filtrate was concentrated to form a pale orange solid. (Ty=564 mg). The product was carried onto the following step without further purification.

In a similar fashion the following compounds were synthesized.

| Example | Structure | Name | Yield |
|---|---|---|---|
| 7.2 | 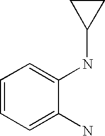 | N-Cyclopropyl-benzene-1,2-diamine | Crude product was used in the later steps without further purification. |
| 7.3 | 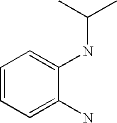 | N-Isopropyl-benzene-1,2-diamine | Crude product was used in the later steps without further purification. |
| 7.4 | 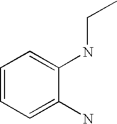 | N-Ethyl-benzene-1,2-diamine | Crude product was used in the later steps without further purification. |
| 7.5 | 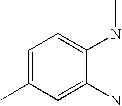 | 4,N-1-Dimethyl-benzene-1,2-diamine | Crude product was used in the later steps without further purification. |

### Example 8.1

### 4-Chloro-N-2-methyl-benzene-1,2-diamine

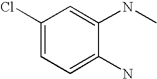

Ammonium chloride (598 mg, 11.2 mmol) and ferrum (4.8 g, 86.3 mmol) were placed in a flask and added with deionized water and set stirring under reflux conditions for 15 minutes. (5-Chloro-2-nitro-phenyl)-methyl-amine (3.2 g, 17.2 mmol) was added to the reaction and the reaction was allowed to reflux for 30 min to 4 hours. The reaction was cooled to room temperature and the pH was adjusted to ˜7 using 5% sodium bicarbonate solution. The reaction mixture was filtered through a pad of diatomaceous earth to remove the ferrum. The filtrate was extracted three times with ethyl acetate. The organic phases were washed with 5% HCl solution. The aqueous was neutralized using 20% sodium hydroxide solution and extracted three times with ethyl acetate. The organic phases were dried over anhydrous sodium sulfate, filtered and concentrated. The products (brown oil) were carried onto the following step without further purification. (2.42 g, ˜90%)

¹HNMR (300 MHz, CDCl₃): (ppm) 2.795 (s, 3H, N—CH₃), 3.415 (br, 3H, NH), 6.561-6.710 (m, 3H, H—Ar).

In a similar fashion the following compounds were synthesized.

| Example | Structure | Name | Yield |
|---|---|---|---|
| 8.2 | 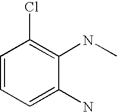 | 3-Chloro-N-2-methyl-benzene-1,2-diamine | Crude product was used in the later steps without further purification. |
| 8.3 | 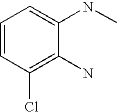 | 3-Chloro-N-1-methyl-benzene-1,2-diamine | Brown oil, (220 mg, 56%) |
| NMR | 2.754 (s, 3 H, N—CH₃), 3.771 (br, 3 H, NH), 6.597 (s, 1 H, H—Ar), 6.869 (s, 2 H, | | |
| | H—Ar) | | |
| 8.4 | 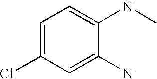 | 4-Chloro-N-1-methyl-benzene-1,2-diamine | Dark purple oil, (2.02 g, 76%) |

| | |
|---|---|
| NMR | 2.837 (s, 3 H, N—CH₃), 3.376 (br, 3 H, N—H, 6.570 (d, 1 H, H—Ar). 6.694 (d, 1 H, |
| | H—Ar), 7.839 (d, 1 H, H—Ar) |

### Example 9.1

### 4,N-2-Dimethyl-benzene-1,2-diamine

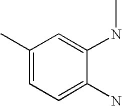

Methyl-(5-methyl-2-nitro-phenyl)-amine (500 mg, 3.048 mmol) dissolved in ethanol (10 m L). Palladium on carbon (5%, 500 mg) was added to the flask; the flask was affixed with a balloon filled with hydrogen and set stirring at room temperature. The reaction was allowed to stir for ˜24 hours. The reaction was filtered through a pad of diatomaceous earth. The filtrate was concentrated to give brown oil. The product was carried onto the next step unpurified. (Ty=415 mg).

¹HNMR (300 MHz, CDCl₃): (ppm) 1.412 (s, 3H, N—CH₃), 6.321 (s, 1H, H—Ar), 6.370 (d, 1H, H—Ar), 6.457 (d, 1H, H—Ar)

In a similar fashion the following compounds were synthesized.

| Example | Structure | Name | Yield |
|---|---|---|---|
| 9.2 | 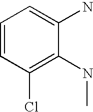 | 3-Chloro-N-2-methyl-benzene-1,2-diamine | Dark brown oil, crude product was used in the later steps without further purification. |

### Example 10.1

### 2-Chloromethyl-1,7-dimethyl-1H-benzoimidazole

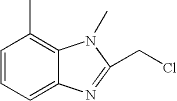

3,N-2-dimethyl-benzene-1,2-diamine (564 mg, 4.14 mmol) and chloroacetic acid (585.9 mg, 6.2 mmol) were dissolved in 50 mL 6M hydrochloric acid. The reaction was heated to reflux and allowed to react from 12 to 24 hours. The reaction was allowed to cool in an ice bath where the reaction was basified using sodium bicarbonate. The reaction was diluted with water and extracted with ethyl acetate. The organic layer was washed three times with water and then with brine. The organic phase was dried over anhydrous sodium sulfate and concentrated in vacuo. The crude product was purified by column chromatography using acetone:dichloromethane=10%:90% to give product. (135 mg, 16.7%)

¹HNMR (300 MHz, CDCl₃): (ppm) 2.747 (s, 3H, C—CH₃), 4.074 (s, 3H, N—CH₃), 4.815 (s, 2H, ═C—CH₂—Cl), 7.013 (d, 1H, H—Ar), 7.154 (t, 1H, H—Ar), 7.607 (d, 1H, H—Ar).

In a similar fashion the following compounds were synthesized.

| Example | Structure | Name | Yield |
|---|---|---|---|
| 10.2 | 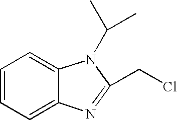 | 2-Chloromethyl-1-isopropyl-1H- benzoimidazole | Pink solid (88%) |
| NMR | 8.17 (m, 1 H), 7.87 (m, 1 H), 7.68 (m, 2 H), 5.33 (s, 1 H), 5.2 (m, 1 H), 1.83 (d, 6 H) | | |
| 10.3 | 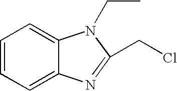 | 2-Chloromethyl-1-ethyl-1H- benzoimidazole | White solid (46%) |

| | |
|---|---|
| NMR | 7.77 (d, 1 H), 7.34 (m, 3 H), 4.87 (s, 2 H), 4.34 (q, 2 H), 1.54 (t, 3 H) |

### Example 11.1

### 5-Chloro-2-chloromethyl-1-methyl-1H-benzoimidazole

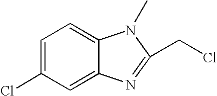

4-Chloro-N-1-methyl-benzene-1,2-diamine (100 mg, 0.64 mmol) was dissolved in 5 mL of 2-chloro-1,1,1-trimethoxy-ethane, and 80 uL of 12N HCl was added to the reaction. The reaction was allowed to stir at room temperature for overnight. The reaction was poured onto saturated sodium bicarbonate solution and extracted with dichloromethane. The combined organic layers were dried over anhydrous sodium sulfate, filtered and concentrated. The crude products were purified by column chromatography using acetone:dichloromethane 10%:90% to give white solid (129.9 mg, 93.9%).

¹HNMR (300 MHz, CDCl₃): (ppm) 3.80 (s, 3H, N—CH₃), 4.80 (s, 2H, ═C—CH₂—Cl, 7.21-7.28 (m, 1H, H—Ar), 7.70 (s, 1H H—Ar).

In a similar fashion the following compounds were synthesized.

| Example | Structure | Name | Yield |
|---|---|---|---|
| 11.2 | 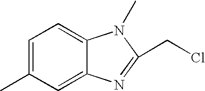 | 2-Chloromethyl-1,5-dimethyl-1H- benzoimidazole | Pale yellow solid, 30% |
| 11.3 | 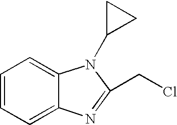 | 2-Chloromethyl-1-cyclopropyl-1H- benzoimidazole | pale yellow solid (542.3 mg, 90.5%). |
| NMR | 1.188-1.211 (m, 2 H, C—CH₂—C), 1.289-1.320 (m, 2 H, C—CH₂—C), 3.351-3.424 (m, | | |
| | 1 H, N—CH—C₂), 4.941 (s, 2 H, ═C—CH₂—Cl), 7.282-7.338 (m, 2 H, H—Ar), | | |
| | 7.567-7.773 (d of d, 2 H, H—Ar | | |
| 11.4 | 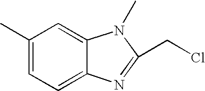 | 2-Chloromethyl-1,6-diinethyl-1H- benzoimidazole | pale yellow solid (92.7%) |
| NMR | 2.163 (s 3 H, C—CH₃), 3.794 (s, 3 H, N—CH₃), 4.805 (s, 2 H, ═C—CH₂—Cl), | | |
| | 7.095 (d, 2 H, H—Ar), 7.611 (d, 1 H, H—Ar). | | |
| 11.5 | 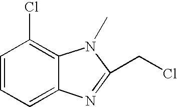 | 7-Chloro-2-chloromethyl-1-methyl- 1H-benzoimidazole | red oil (70.2 mg, 12.2%). |
| NMR | 4.163 (s, 3 H, N—CH₃), 4.803 (s, 2 H, ═C—CH₂—Cl), 7.136-7.227 (t, 1 H, H—Ar), | | |
| | 7.253-7.256 (d, 1 H, H—Ar), 7.616-7.646 (d of d, 1 H, H—Ar). | | |
| 11.6 | 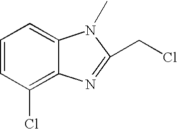 | 4-Chloro-2-chloromethyl-1-methyl- 1H-benzoimidazole | pale yellow solid (253 mg, 84.1%) |
| NMR | 4.163 (s, 3 H, N—CH₃), 4.803 (s, 2 H, C—CH₂—Cl), 7.136-7.227 (t, 1 H, H—Ar), | | |
| | 7.253-7.256 (d, 1 H, H—Ar, 7.616-7.646 (d of d, 1 H, H—Ar). | | |
| 11.7 | 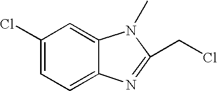 | 6-Chloro-2-chloromethyl-1-methyl- 1H-benzoimidazole | white solid (129.9 mg, 93.9%) |

| | |
|---|---|
| NMR | 3.841 (s, 3 H, N—CH₃), 4.835 (s, 2 H, ═C—CH₂—Cl), 7.220-7.362 (m, 2 H, H—Ar), |
| | 7.631-7.660 (d, 1 H, H—Ar) |

### Example 12

### (1-Methyl-1H-benzoimidazol-2-yl)-methanol

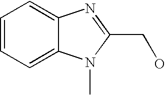

Sodium borohydride (472 mg, 12.48 mmol) was added to the solution of 1-methyl-1H-benzoimidazole-2-carbaldehyde (1 g, 6.24 mmol) in ethanol (50 mL). The reaction mixture was stirred at room temperature for overnight. The reaction mixture was condensed, the residue was diluted with ethyl acetate; water was added. The organic layer was washed with water and brine, dried over anhydrous sodium sulfate, filtered and condensed to give off-white solid as product (953 mg, 94%). This product was used in the later steps without further purification.

¹HNMR (300 MHz, CDCl₃): (ppm) 7.63 (m, 1H), 7.21 (m, 3H), 4.84 (s, 2H), 3.75 (s, 3H)

### Example 13

### 2-Chloromethyl-1-methyl-1H-benzoimidazole

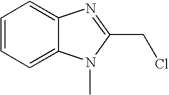

(1-Methyl-1H-benzoimidazol-2-yl)-methanol (330 mg, 2.03 mmol) was dissolved in dichloromethane (15 mL), thionyl chloride (1.5 mL) was added drop wise. The reaction mixture was stirred at room temperature for overnight. The solvent was then removed from the reaction mixture by concentration in vacuo. The residue was dried on vacuum pump. Yellow solid was obtained as product (520 mg, yield: quantity).

¹HNMR (300 MHz, CDCl₃): (ppm) 7.98 (br, 1H), 7.86 (br, 1H), 7.72 (m, 2H), 5.32 (s, 2H), 4.15 (s, 3H)

### Example 14

### 4-(2-Bromo-ethyl)-piperidine-1-carboxylic Acid tert-butyl Ester

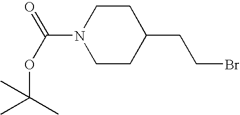

A solution of triphenyl-phosphane (1.716 g, 6.54 mmol) in dichloromethane was added slowly over an hour to the solution of 4-(2-hydroxy-ethyl)-piperidine-1-carboxylic acid tert-butyl ester and tetrabromomethane in dichloromethane (20 mL) at room temperature. The reaction mixture was stirred at room temperature for overnight. The reaction mixture was diluted with hexane (50 mL), and then washed with water and brine. The organic phase was dried over anhydrous sodium sulfate, filtered and condensed to dryness. The crude residue was purified on silica gel using ether:hexane=10%:90% then ether:hexane=30%:70% to give the product as colorless oil (1.67 g, 87%).

¹HNMR (300 MHz, CDCl₃): (ppm) 4 (br, 2H), 3.35 (t, 2H), 2.58 (br, 2H), 1.7 (q, 2H), 1.59 (br, 3H), 1.35 (s, 9H), 1.02 (br, 2H)

### Example 15.1

### 4-[2-(4-Fluoro-phenoxy)-ethyl]-piperidine-1-carboxylic Acid tert-butyl Ester

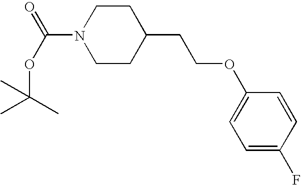

4-Fluoro-phenol (230 mg, 2.05 mmol), potassium carbonate (1.11 g, 8.04 mmol), tetrabutyl ammonium iodide (45 mg, 0.123 mmol) were added to the solution of 4-(2-bromo-ethyl)-piperidine-1-carboxylic acid tert-butyl ester (600 mg, 2.05 mmol) in acetone. The reaction mixture was refluxed for overnight. The reaction mixture was concentrated in vacuo; the residue was partitioned between ethyl acetate and water. Organic layer was washed with 1N sodium hydroxide aqueous solution (3×20 mL), water and brine. The organic phase was dried over anhydrous sodium sulfate; filtered and condensed to dryness. Yellow oil was obtained as product (700 mg, 98%). This product was used directly in the subsequent step to generate the corresponding amine in situ, which reacted with 2-Chloromethyl-1-methyl-1H-benzoimidazole in Example 13 to give the final compound 2-{4-[2-(4-Fluoro-phenoxy)-ethyl]-piperidin-1-ylmethyl}-1-methyl-1H-benzoimidazole (Example 1.1).

¹HNMR (300 MHz, CDCl₃): (ppm) 6.94 (t, 2H), 6.77 (m, 2H), 4 (br, 2H), 3.9 (t, 2H), 2.62 (br, 2H), 1.61 (br, 5H), 1.43 (s, 9H), 1.16 (br, 2H)

In a similar fashion the following compounds were synthesized.

| Example | Structure | Name | Yield |
|---|---|---|---|
| 15.2 | 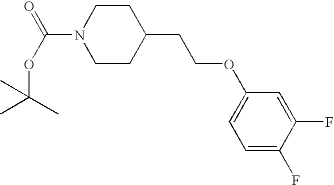 | 4-[2-(3,4-Difluoro-phenoxy)-ethyl]- piperidine-1-carboxylic acid tert- butyl ester | Yellow oil 93.5% |
| NMR | 6.98 (q, 1 H), 6.62 (m, 1 H), 6.48 (m, 1 H), 4.12 (br, 2 H), 3.94 (t, 2 H), 2.65 (br, 2 H), 1.71 (br, | | |
| | 5 H), 1.46 (s, 9 H), 1.08 (br, 2 H) | | |
| 15.3 | 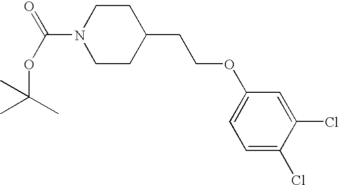 | 4-[2-(3,4-Dichloro-phenoxy)-ethyl]- piparidine-1-carboxylic acid tert- butyl ester | 116% |

| | |
|---|---|
| NMR | 7.27 (d, 1 H), 6.94 (d, 1 H), 6.7 (dd, 1 H), 4.08 (br, 2 H), 3.93 (t, 2 H), 2.69 (br, 2 H), 1.68 (br, 5 H), |
| | 1.49 (s, 9 H), 1.16 (br, 2 H) |

The above products (Example 15.2 to Example 15.3) were used directly in the subsequent step to generate the corresponding amines in situ, which reacted with suitable halogenated intermediates listed above in Example 10, 11, 13, 14, to give the final compounds in Example 1.

### Example 16.1

### 4-[2-(4-Fluoro-phenoxy)-ethyl]-piperidine Hydrochloride

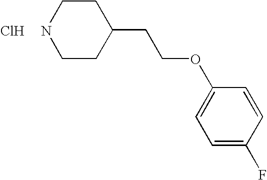

4-[2-(4-Fluoro-phenoxy)-ethyl]-piperidine-1-carboxylic acid tert-butyl ester (700 mg) was dissolved in diethylether; 1M hydrochloride in diethylether (5 mL) was added to the above solution. The reaction mixture was stirred at room temperature for 30 min, and then filtered. The precipitation was washed with diethylether. White solid (520 mg) was obtained as product.

In a similar fashion the following compounds were synthesized.

| Example | Structure | Name | Yield |
|---|---|---|---|
| 16.2 | 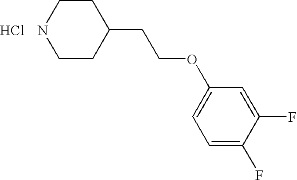 | 4-[2-(3,4-Difluoro-phenoxy)-ethyl]- piperidine hydrochloride | White solid yield: N.A |
| 16.3 | 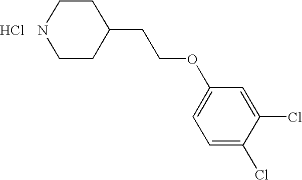 | 4-[2-(3,4-Dichloro-phenoxy)-ethyl]- piperidine hydrochloride | White solid, yield: N.A. |

### Example 17.1

### 3-Allyl-piperidine-1-carboxylic Acid tert-butyl Ester

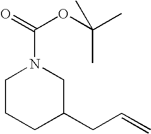

Methyl-triphenyl-lambda-5-phosphonium bromide (1.57 g, 4.4 mmol) and DBU (670 mg, 4.4 mmol) were added to the solution of 3-formyl-piperidine-1-carboxylic acid tert-butyl ester (500 mg, 2.2 mmol) in acetonitrile (5 mL). The reaction mixture was refluxed for overnight. After removing acetonitrile in vacuo, the residue was partitioned between ethyl acetate and water. The organic layer was washed with brine, dried over anhydrous sodium sulfate, filtered and condensed in vacuo. The crude residue was purified on silica gel using ethyl acetate:hexane=20%:80% to give the product as yellow oil (278 mg, 58%)

¹HNMR (300 MHz, CDCl₃): (ppm) 5.73 (m, 1H), 4.99 (m, 2H), 3.88 (br, 2H), 2.78 (br, 1H), 2.4 (br, 1H), 1.95 (q, 2H), 1.8 (br, 1H), 1.58 (br, 1H), 1.52 (br, 1H), 1.42 (br, 1H), 1.38 (s, 9H), 1.04 (br, 1H)

In a similar fashion the following compounds were synthesized.

| Example | Structure | Name | Yield |
|---|---|---|---|
| 17.2 | 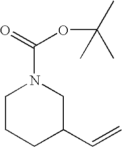 | 3-Vinyl-piperidine-1-carboxylic acid tert- butyl ester | colorless oil, 54% |
| NMR | 5.68 (m, 1 H), 4.98 (m, 2 H), 3.9 (br, 2 H), 2.67 (td, 2 H), 2.06 (br, 1 H), 1.76 (br, | | |
| | 1 H), 1.58 (br, 1 H), 1.39 (br, 1 H), 1.38 (s, 9 H), 1.19 (br, 1 H) | | |
| 17.3 | 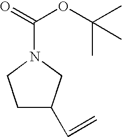 | 3-Vinyl-pyrrolidine-1-carboxylic acid tert- butyl ester | Colorless oil, 53% |

| | |
|---|---|
| NMR | 5.64 (m, 1 H), 4.98 (m, 2 H), 3.45 (m, 2 H), 3.22 (m, 1 H), 3.09 (m, 1 H), 2.69 (m, |
| | 1 H), 1.98 (m, 1 H), 1.42 (m, 1 H), 1.4 (s, 9 H) |

These products (Example 17.1 to Example 17.3) were used directly in the subsequent step to generate the corresponding amines in situ, which reacted with various halogenated intermediates listed above in Example 10, 11, 13, 14, to give the final compounds in Example 1.

### Example 18.1

### 3-[3-(4-Fluoro-phenyl)-propyl]-piperidine-1-carboxylic Acid tert-butyl Ester

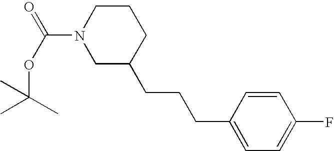

3-Allyl-piperidine-1-carboxylic acid tert-butyl ester (160 mg, 0.71 mmol) was charged into a screwed vial. After degassed and back-filled with argon, 9-BBN was added through syringe. The reaction mixture was stirred at 60° C. for one hour. After cooling to room temperature, it was added to the mixture of 1-bromo-4-fluoro-benzene (150 mg, 0.86 mmol), potassium carbonate and Pd(dppf)Cl₂ in N,N-dimethylformamide (2 mL) with water (0.2 mL). The resulting mixture was stirred at 90° C. for 36 hours. The reaction mixture was cooled to room temperature, diluted with water, extracted with ethyl acetate. The organic phase was washed with water and brine, and dried over anhydrous sodium sulfate, filtered and condensed in vacuo. The crude residue was purified on silica gel using ethyl acetate:hexane=10%:90% to give the product (165 mg, 72%).

¹HNMR (300 MHz, CDCl₃): (ppm) 7.14 (m, 2H), 6.95 (t, 2H), 3.82 (br, 2H), 2.79 (br, 1H), 2.58 (t, 2H), 2.4 (br, 1H), 1.85 (m, 2H), 1.62 (m, 4H), 1.46 (sum, 9H), 1.2 (br, 1H), 1.04 (m, 2H)

In a similar fashion the following compounds were synthesized.

| Example | Structure | Name | Yield |
|---|---|---|---|
| 18.2 | 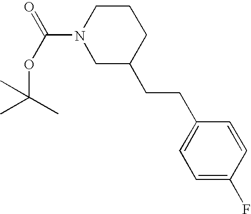 | 3-[2-(4-Fluoro- phenyl)-ethyl]- piperidine-1- carboxylic acid tert- butylester | Yellow oil 84% |
| NMR | 7.12 (m, 2 H), 6.95 (t, 2 H), 3.83 (br, 2 H), 2.81 (br, 1 H), 2.61 (t, 2 H), 2.38 (m, 1 H), 1.86 (br, | | |
| | 2 H), 1.52 (br, 4 H), 1, 48 (s, 9 H), 1.12 (br, 1 H) | | |
| 18.3 | 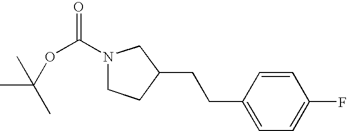 | 3-[2-(4-Fluoro- phenyl)-ethyl]- pyrrolidine-1- carboxylic acid tert- butyl ester | Yellow oil 88% |
| NMR | 7.28 (m, 2 H), 6.98 (t, 2 H), 3.45 (m, 2 H), 2.89 (m, 1 H), 2.49 (t, 1 H), 2.38 (m, 2 H), 1.86 (m, 4 H), | | |
| | 1.52 (m, 10 H) | | |
| 18.4 | 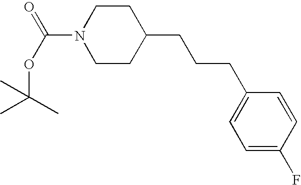 | 4-[3-(4-Fluoro- phenyl)-propyl]- piperidine-1- carboxylic acid tert- butyl ester | Yellow oil 73% |
| NMR | 7.10 (m, 2 H), 6.95 (m, 2 H), 4.06 (m, 2 H), 2.57 (m, 4 H), 1.62 (m, 4 H), 1.45 (s, 9 H), 1.25 (m, | | |
| | 3 H), 1.00 (m, 2 H) | | |
| 18.5 | 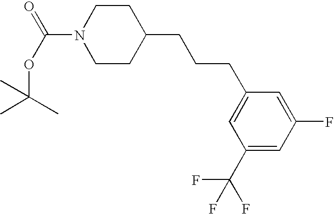 | 4-[3-(3-Fluoro-5- trifluoromethyl- phenyl)-propyl]- piperidine-1- carboxylic acid tert- butyl ester | Yellow oil 91% |
| NMR | 7.19 (m, 1 H), 7.00-7.15 (m, 2 H), 4.07 (br, 2 H), 2.59-2.70 (m, 4 H), 1.49-1.64 (m, 4 H), 1.42 (s, | | |
| | 9 H), 1.24-1.34 (m, 3 H), 0.86-1.06 (m, 2 H) | | |
| 18.6 | 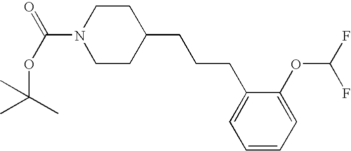 | 4-[3-(2- Difluoromethoxy- phenyl)-propyl]- piperidine-1- carboxylic acid tert- butyl ester | Yellow oil 16% |
| NMR | 7.17 (m, 4 H), 6.5 (t, 1 H), 4.17 (br, 2 H), 2.64 (m, 4 H), 1.88 (m, 2 H), 1.58 (m, 5 H), 1.46 (s, 9 H), | | |
| | 1.04 (m, 2 H) | | |
| 18.7 | 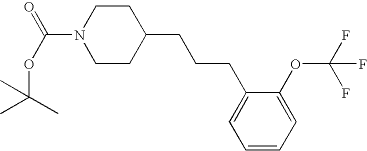 | 4-[3-(2-Trifluoro- methoxy-phenyl)- propyl]-piperidine-1- carboxylic acid tert-butyl ester | Yellow oil 28% |
| 18.8 | 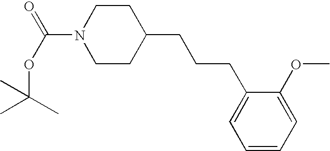 | 4-[3-(2-Methoxy- phenyl)-propyl]- piperidine-1- carboxylic acid tert- butyl ester | Colorless oil 67% |
| 18.9 | 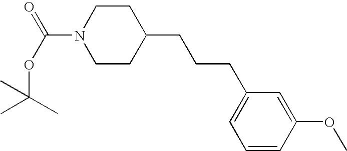 | 4-[3-(3-Methoxy- phenyl)-propyl]- piperidine-1- carboxylic acid tert- butyl ester | Colorless oil 21% |
| 18.10 | 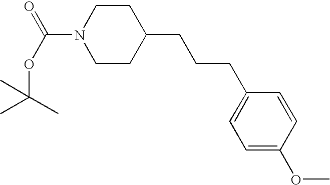 | 4-[3-(4-Methoxy- phenyl)-propyl]- piperidine-1- carboxylic acid tert- butyl ester | Colorless oil 55% |
| 18.11 | 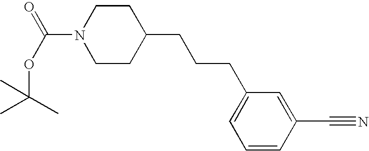 | 4-[3-(3-Cyano- phenyl)-propyl]- piperidine-1- carboxylic acid tert- butyl ester | Yellow oil 85% |
| NMR | 7.36 (m, 4 H), 4 (br, 2 H), 2.6 (t, 4 H), 1.82 (m, 2 H), 1.61 (m, 3 H), 1.41 (s, 9 H), 1.37 (m, 2 H), | | |
| | 1.21 (m, 2 H) | | |
| 18.12 | 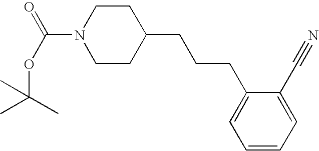 | 4-(3-(2-Cyano- phenyl)-propyl]- piperidine-1- carboxylic acid tert- butyl ester | Yellow oil 64% |
| NMR | 7.6 (d, 1 H), 7.57 (td, 1 H), 7.27 (m, 2 H), 4.15 (br, 2 H), 2.83 (td, 2 H), 2.78 (br, 2 H), 1.82 (m, | | |
| | 2 H), 1.66 (m, 3 H), 1.43 (s, 9 H), 1.34 (m, 2 H), 1.21 (m, 2 H) | | |
| 18.13 | 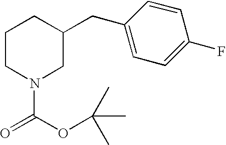 | 3-(4-Fluoro-benzyl)- piperidine-1- carboxylic acid tert- butyl ester | Yellow oil, 79% |
| NMR | 7.09 (dd, 2 H), 6.95 (td, 2 H), 3.87 (br, 2 H), 2.4 (m, 4 H), 1.88 (m, 2 H), 1.62 (m, 3 H), 1.41 (s, | | |
| | 9 H) | | |
| 18.14 | 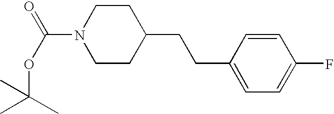 | 4-[2-(4-Fluoro- phenyl)-ethyl]- piperidine-1- carboxylic acid tert- butyl e | Yellow solid |

These products (Example 18.1 to Example 18.14) were used directly in the subsequent step to generate the corresponding amines in situ, which reacted with various halogenated intermediates listed above in Example 10, 11, 13, 14, to give the final compounds in Example 1.

### Example 19.1

### 4-Allyl-piperidine-1-carboxylic Acid tert-butyl Ester

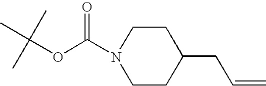

To a suspension of methyl triphenylphosphonium bromide (2.6 g, 7.27 mmol) in tetrahydrofuran (20 mL), butyllithium (2M in hexane, 3.63 mL, 7.27 mmol) was added dropwise at −78° C. The reaction mixture was stirred at −78° C. for one hour. The solution of 4-(2-oxo-ethyl)-piperidine-1-carboxylic acid tert-butyl ester (1.5 g, 6.6 mmol) in tetrahydrofuran (20 mL) was added to the above reaction mixture. The resulting mixture was allowed to warm to room temperature and stirred for overnight. The reaction mixture was diluted with ethyl acetate (30 mL), washed with water and brine. The organic phase was dried over anhydrous sodium sulfate, filtered and concentrated in vacuo. The crude residue was purified on silica gel using ethyl acetate:hexane=20%:80% to give the product as colorless oil (550 mg, 37%).

¹HNMR (300 MHz, CDCl₃): (ppm) 5.65 (m, 1H), 4.92 (m, 2H), 3.98 (br, 2H), 2.55 (br, 2H), 1.9 (t, 2H), 1.53 (br, 2H), 1.36 (s, 9H), 0.98 (m, 2H), 0.79 (m, 1H)

In a similar fashion the following compounds were synthesized.

| Example | Structure | Name | Yield |
|---|---|---|---|
| 19.2 | 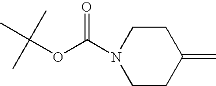 | 4-Methylene-piperidine-1-carboxylic acid tert-butyl ester | Colorless oil 99% |

| | |
|---|---|
| NMR | 4.63 (s, 2 H), 3.13 (t, 4 H), 2.07 (t, 4 H), 1.37 (s, 9 H) |

These products (Example 19.1 to Example 19.2) were used directly in the subsequent step to generate the corresponding amines in situ, which reacted with various halogenated intermediates listed above in Example 10, 11, 13, 14, to give the final compounds in Example 1.

### Example 20

### 3-Oxo-4-(3-phenyl-propyl)-piperazine-1-carboxylic Acid tert-butyl Ester

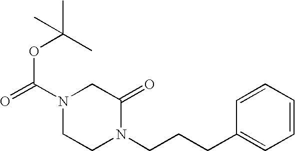

Sodium tert-butoxide (70 mg, 0.72 mmol) was added to the solution of 3-oxo-piperazine-1-carboxylic acid tert-butyl ester (120 mg, 0.6 mmol) and (3-bromo-propyl)-benzene (143 mg, 0.72 mmol) in N,N-dimethylformamide (5 mL). The reaction mixture was stirred at room temperature for over night. The reaction mixture was diluted with water. Product was extracted with ethyl acetate. Organic phase was washed with water and brine, then dried over anhydrous sodium sulfate and concentrated in vacuo. The crude residue was purified on silica gel using ethyl acetate:hexane=40%:60% to give the product as colorless oil (143 mg, 75%). This product was used directly in the subsequent step to generate the corresponding amines in situ, which reacted with various halogenated intermediates listed above in Example 10, 11, 13, 14, to give the final compounds in Example 1.

¹HNMR (300 MHz, CDCl₃): (ppm) 7.27 (m, 2H), 7.17 (m, 3H), 4.03 (s, 2H), 3.55 (t, 2H), 3.45 (t, 2H), 3.26 (t, 2H), 2.63 (t, 2H), 1.9 (m, 2H), 1.45 (s, 9H)

### Example 21

### 4,4-Diphenyl-piperidine

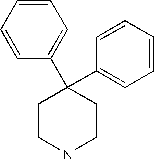

4,4-Diphenyl-piperidine was synthesized from piperidine-4,4 diol (1.0 g, 8.536 mmol), and an excess of TfOH (10 mL), and benzene (10 mL). The reaction was stirred at room temperature for 4 hours. The reaction was poured onto ice. The solution was made basic using 1M NaOH and then extracted with dichloromethane. The organic layer was washed with water and then brine. The organic layer was dried over Na₂SO₄, filtered and concentrated to give a white solid (1.49 g, 96.8%).

¹HNMR (300 MHz, CDCl₃): (ppm) 2.664 (t, 4H, 2(C—CH₂—C), 3.219 (t, 4H, 2(N—CH₂—C), 7.200-7.384 (m, 10H, H—Ar).

### Example 22

### tert-Butyl 4-[(1-methyl-7-methyl-1H-benzimidazol-2-yl)methyl]piperidine-1-carboxylate

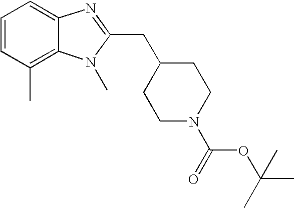

3,N-2-Dimethyl-benzene-1,2-diamine (204.3 mg, 1.5 mmol) was dissolved in ethanol (10 mL). Palladium on carbon (100 mg) was added follow by 4-(2-Oxo-ethyl)-piperidine-1-carboxylic acid tert-butyl ester (376 mg, 1.65 mmol). The reaction mixture was refluxed for 3 days. The reaction was then filtered through diatomaceous earth pad and the filtrate was concentrated in vacuo. The crude residue was purified on silica gel using ethyl acetate:hexane=40%:60%, then 2M ammonia in methanol:ethyl acetate=5%:95% to give the product as yellow gum (330 mg, 64%).

¹HNMR (300 MHz, CDCl₃): (ppm) 7.57 (d, 1H), 7.11 (t, 1H), 6.97 (d, 1H), 3.98 (s, 3H), 2.82 (d, 2H), 2.8 (s, 3H), 2.76 (br, 2H), 2.06 (br, 1H), 1.7 (br, 4H), 1.46 (s, 9H), 1.28 (br, 2H)

### Example 23

### 1,7-Dimethyl-2-piperidin-4-ylmethyl-1H-benzoimidazole

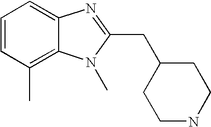

To the solution of tert-butyl 4-[(1-methyl-7-methyl-1H-benzimidazol-2-yl)methyl]piperidine-1-carboxylate (330 mg, 0.77 mmol) in dichloromethane was added the 1:1 mixture of trifluoroacetic acid (2.5 mL) and dichloromethane (2.5 mL) at room temperature. The reaction mixture was stirred at room temperature for one hour. Diluted the reaction mixture with chloroform, and quenched with saturated sodium bicarbonate aqueous solution until pH=9˜10. The product was extracted with chloroform. Organic phase was washed with water and brine, dried over anhydrous sodium sulfate, filtered and concentrated in vacuo to give the product as yellow gum (160 mg, 68.5%).

¹HNMR (300 MHz, CDCl₃): (ppm) 7.57 (d, 1H), 7.12 (t, 1H), 6.95 (d, 1H), 3.98 (s, 3H), 3.1 (br, 2H), 2.82 (d, 2H), 2.76 (s, 3H), 2.62 (td, 2H), 1.89 (m, 1H), 1.75 (br, 2H), 1.31 (m, 2H).

### Example 24

### 2-(4-Benzyl-piperidin-1-ylmethyl)-1H-benzimidazole

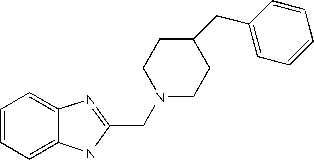

To a stirred solution of 2-chloromethyl-1H-benzimidazole (1.0 g, 6.0 mmol) in DMF (10 mL) was added 4-benzyl-piperidine (1.0 g, 6.0 mmol) and the reaction was heated to 110° C. for 6 h. The reaction mixture was poured into water and then extracted with ethyl acetate (100 mL). The ethyl acetate was dried (Na₂SO₄), filtered and concentrated in vacuo. The residual oil was chromatographed (SiO₂, EtOAc, Hex) to give the title compound as a white solid (0.45 g, 25%). APCI, m/z=306 (M+1).

### Example 25.1

### 2-(4-Benzyl-piperidin-1-ylmethyl)-1-(4-bromo-benzyl)-1H-benzimidazole

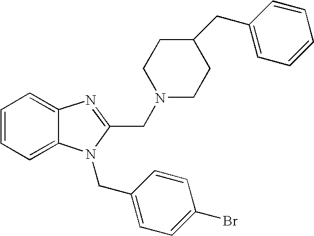

To a stirred solution of 2-(4-Benzyl-piperidin-1-ylmethyl)-1H-benzoimidazole (title compound in Example 24) (0.14 g, 0.5 mmol) in DMF was added NaH (0.019 g, 1 equiv, 60% min oil). The reaction was stirred for five minutes, whereupon 4-bromobenzylchloride was added. The reaction was stirred for 2 hours and then poured into water. The aqueous layer was extracted with ethyl acetate and the organic layer was concentrated. Purification by (SiO₂, EtOAc, Hex) gave rise to pure material (200 mg, 85%) as a white solid.

¹H NMR (300 MHz, DMSO-d6): δ 1.08 (m, 2H), 1.45 (m, 3H), 2.04 (m, 2H), 2.47 (m, 2H, J=6.6 Hz), 2.73-2.77 (m, 2H), 3.68 (s, 2H), 5.52 (s, 2H), 6.97 (d, 2H, J=8.4 Hz), 7.10 (d, 1H, J=6.6 Hz), 7.22 (m, 8H), 7.46 (d, 1H, J=8.4 Hz), 7.73 (d, 1H, J=8.4 Hz). APCI, m/z=476, 477 (M+1).

In a similar fashion the following compounds were synthesized.

| Example | Structure | Name | Yield |
|---|---|---|---|
| 25.2 | 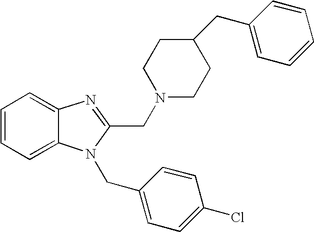 | 2-(4-Benzyl-piperidin-1- ylmethyl)-1-(4-chloro- benzyl)-benzimidazole | |

| | |
|---|---|
| NMR | (300 MHz, DMSO-d6) δ 1.08 (m, 2 H), 1.45 (m, 3 H), 2.04 (m, 2 H), 2.47 (m, 2 H, |
| | J = 6.6 Hz), 2.73-2.77 (m, 2 H), 3.68 (s, 2 H), 5.52 (s, 2 H), 6.90 (d, 2 H, J = 8.4 |
| | Hz), 7.04 (d, 1 H, J = 6.6 Hz), 7.22 (m, 10 H), 7.73 (d, 1 H, J = 8.4 Hz) |

### Example 26.1

### 2-[1-(4-Benzyl-piperidin-1-yl)-ethyl]-1-methyl-1H-benzoimidazole

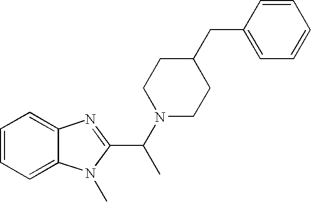

A suspension of 1-(1H-Benzoimidazol-2-yl)-ethanol (0.25 g, 1.54 mmol) in CHCl₃ (2 mL) was treated with thionyl chloride (1 mL) and heated to 60 C for 1 h until the solution becomes clear. The solvent was removed at reduced pressure to give an oil. This oil was taken up in CH₂Cl₂ (2 mL) treated with diisopropylethylamine (0.6 mL, 3.4 mmol) and then with 4-benzylpiperidine (0.27 mL, 1.54 mmol) and stirred at room temperature for 2 hrs. The reaction was the diluted with EtOAc, washed with saturated NH₄Cl, the organic layer separated, dried (MgSO₄) and the solvent removed at reduced pressure to give yellow oil. This oil was suspended dissolved in DMF (1 mL) and treated with sodium hydride (89 mg, 2.3 mmol). After 10 min the reaction was treated with methyl iodide (0.11 mL, 1.7 mmol) and stirred at room temperature for 1 h. The reaction was poured into water, extracted with EtOAc, the organic layer separated, dried (MgSO₄) and the solvent removed at reduced pressure. Chromatography (silica, 0 to 10% MeOH/DCM) gave the product as an off white solid. (0.25 g, 55%)

LC/MS 2.11 min.: 334 (M+H, 100%);

¹H-NMR (300 MHz, DMSO-d₆): δ 7.45 (m, 2H), 7.26 (m, 1H), 7.15 (m, 6H), 4.16 (s, 1H), 3.69 (s, 3H), 2.91 (m, 2H), 2.78 (m, 2H), 2.58 (m, 2H), 2.08 (m, 1H), 1.89 (s, 3H), 1.53 (m, 2H), 1.14 (m, 2H).

In a similar fashion the following compounds were synthesized.

| Example | Structure | Name | Yield |
|---|---|---|---|
| 26.2 | 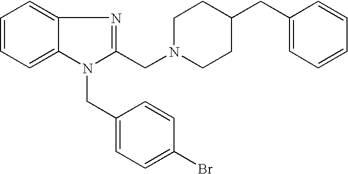 | 2-(4-Benzyl-piperidin-1- ylmethyl)-1-(4-bromo- benzyl)-1H- benzoimidazole | |
| NMR | | | |
| 26.3 | 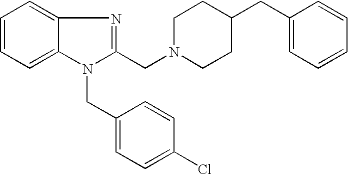 | 2-(4-Benzyl-piperidin-1- ylmethyl)-1-(4-chloro- benzyl)-1H- benzoimidazole | |
| NMR | | | |
| 26.4 | 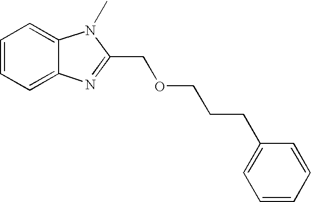 | 1-Methyl-2-(3-phenyl- propoxymethyl)-1H- benzoimidazole | yellow liquid |
| NMR | (300 Mhz, CDCl3) 7.80-7.76 (m, 1 H), 7.41-7.12 (m, 8 H), 4.84 (s, 2 H), 3.90 (s, | | |
| | 3 H), 3.56 (t, J = 6.3 Hz, 2 H), 2.69 (t, J = 7.7 Hz, 2 H), 1.99-1.87 (m, 2 H) | | |
| 26.5 | 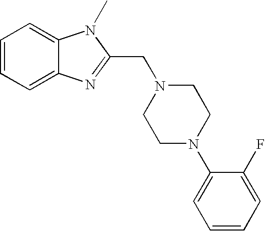 | 2-[4-(2-Fluoro-phenyl)- piperazin-1-ylmethyl]-1- methyl-1H- benzoimidazole | yellow solid |
| NMR | (300 MHz, CDCl3) 7.78-7.72 (m, 1 H). 7.38-7.22 (m, 3 H), 7.06-6.88 (m, 4 H), 3.90 | | |
| | (s, 3 H), 3.89 (s, 2 H), 3.10 (m, 4 H), 2.73 (m, 4 H) | | |
| 26.6 | 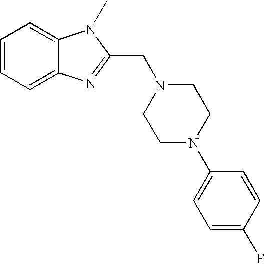 | 2-[4-(4-Fluoro-phenyl)- piperazin-1-ylmethyl)-1- methyl-1H- benzoimidazole | yellow solid |
| NMR | (300 MHz, CDCl3) 7.77-7.72 (m, 1 H), 7.38-7.22 (m, 3 H), 6.99-6.81 (m, 4 H), 3.89 | | |
| | (s, 3 H), 3.88 (s, 2 H), 3.13-3.07 (m, 4 H), 2.73-2.67 (m, 4 H) | | |
| 26.7 | 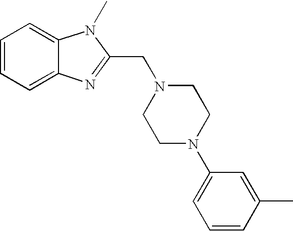 | 1-Methyl-2-(4-m-tolyl- piperazin-1-ylmethyl)-1H- benzoimidazole | yellow solid |
| NMR | (300 MHz, CDCl3) 7.77-7.72 (m, 1 H), 7.37-7.22 (m, 4 H), 7.17-7.09 (m, 1 H), 6.75-6.65 | | |
| | (m, 2 H), 3.89 (s, 3 H), 3.88 (s, 2 H), 3.16 (m, 4 H), 2.69 (m, 4 H), 2.03 (s, 3 H) | | |
| 26.8 | 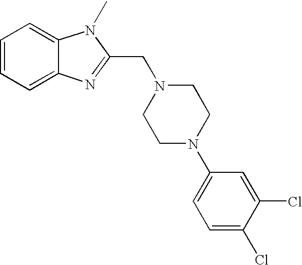 | 2-[4-(3,4-Dichloro- phenyl)-piperazin-1- ylmethyl]-1-methyl-1H- benzoimidazole | yellow solid |
| NMR | (300 MHz, CDCl3) 7.77-7.73 (m, 1 H), 7.37-7.24 (m, 4 H), 6.94-6.91 (m, 1 H), 6.74-6.69 | | |
| | (m, 1 H), 3.89 (s, 3 H), 3.88 (s, 2 H), 3.17-3.13 (m, 4 H), 2.71-2.66 (m, 4 H) | | |
| 26.9 | 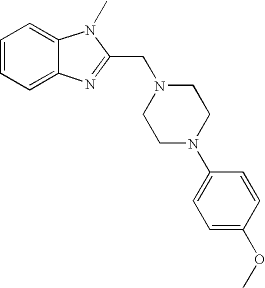 | 2-[4-(4-Methoxy-phenyl)- piperazin-1-ylmethyl]-1- methyl-1H- benzoimidazole | 84%, solid |
| NMR | (300 MHz, CDCl3) 7.77-7.73 (m, 1 H), 7.37-7.23 (m, 3 H), 6.90-6.81 (m, 4 H), 3.89 | | |
| | (s, 3 H), 3.88 (s, 2 H), 3.76 (s, 3 H), 3.10-3.04 (m, 4 H), 2.72-2.68 (m, 4 H) | | |
| 26.10 | 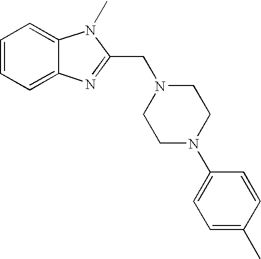 | 1-Methyl-2-(4-p-tolyl- piperazin-1-ylmethyl)-1H- benzoimidazole | 14%, solid |
| NMR | (300 MHz, CDCl3) 7.77-7.73 (m, 1 H), 7.37-7.24 (m, 3 H), 7.06 (d, J = 8.4 Hz, 2 H), | | |
| | 6.82 (d, J = 8.6 Hz, 2 H), 3.90 (s, 3 H), 3.88 (s, 2 H), 3.15 -3.11 (m, 4 H), 2.73-2.67 (m, | | |
| | 4 H), 2.26 (8, 3 H) | | |
| 26.11 | 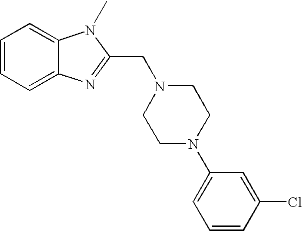 | 2-[4-(3-Chloro-phenyl)- piperazin-1-ylmethyl]-1- methyl-1H- benzoimidazole | 67%, oil |
| NMR | (300 MHz, CDCl3) 7.80-7.72 (m, 1 H), 7.38-7.26 (m, 4 H), 7.19-7.11 (m, 1 H), 6.89-6.74 | | |
| | (m, 2 H), 3.89 (s, 3 H), 3.88 (s, 2 H), 3.21-3.15 (m, 4 H), 2.74-2.66 (m, 4 H) | | |
| 26.12 | 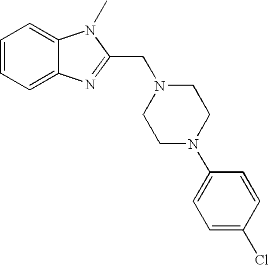 | 2-[4-(4-Chloro-phenyl)- piperazin-1-ylmethyl]-1- methyl-1H- benzoimidazole | 84%, yellow solid |
| NMR | (300 MHz, CDCl3) 7.78-7.74 (m, 1 H), 7.38-7.23 (m, 5 H), 7.19 (d, J = 9.0 Hz, 2 H), | | |
| | 6.82 (d, J = 9.1 Hz, 2 H), 3.89 (s, 3 H), 3.88 (s, 2 H), 3.17-3.11 (m, 4 H), 2.73-2.68 (m, | | |
| | 4 H) | | |
| 26.13 | 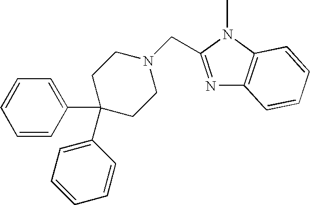 | 2-(4,4-Diphenyl- piperidin-1-ylmethyl)-1- methyl-1H- benzoimidazole | solid |
| NMR | (300 MHz, CDCl3) 7.73-7.70 (m, 1 H), 7.36-7.22 (m, 11 H), 7.17-7.11 (m, 2 H), 3.88 | | |
| | (s, 3 H), 3.73 (s, 2 H), 2.62-2.56 (m, 4 H), 2.47-2.41 (m, 4 H) | | |
| 26.14 | 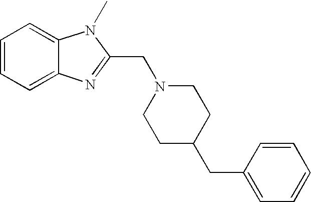 | 2-(4-Benzyl-piperidin-1- ylmethyl)-1-methyl-1H- benzoimidazole | off-white solid |
| NMR | (300 MHz, DMSO) 7.60-7.49 (m, 2 H), 7.27-7.12 (m, 7 H), 3.75-3.71 (m, 2 H), 3.31 | | |
| | (app s, 5 H), 2.85-2.76 (m, 2 H), 2.06-1.95 (m, 2 H), 1.58-1.48 (m, 3 H), 1.26-1.10 | | |
| | (m, 2 H) | | |
| 26.15 | 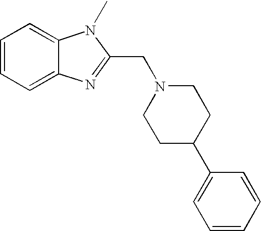 | 1-Methyl-2-(4-phenyl- piperidin-1-ylmethyl)-1H- benzoimidazole | yellow solid |

| | |
|---|---|
| NMR | (300 MHz, DMSO) 7.63-7.49 (m, 2 H), 7.31-7.14 (m, 7 H), 3.85-3.81 (m, 1 H), 3.29 |
| | (app s, 5 H), 3.00-2.93 (m, 2 H), 2.27-2.16 (m, 2 H), 1.77-1.58 (m, 4 H) |

1. The use of a compound according to Formula I: 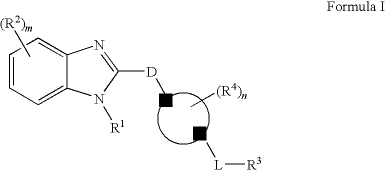
wherein,
A and B are independently selected from the group consisting of N and C, with the proviso that A and B are not both C; 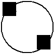
represents a 4- to 8-membered ring;
D is selected from the group consisting of alkylene, alkenylene, and alkynylene;
L is selected from the group consisting of a bond, alkylene, alkenylene, alkynylene, —O—, —X—O—, —O—X—, —X—O—Y, —NR¹⁰—, —X—NR¹⁰—, —NR¹⁰—X—, and —X—NR¹⁰—Y—; wherein X and Y, in each instance, are independently selected from the group consisting of alkylene, alkenylene, and alkynylene, with the proviso that when B is N, L is selected from the group consisting of a bond, alkylene, alkenylene, alkynylene, —X—O—, —X—O—Y—, —X—NR¹⁰—, and —X—NR¹⁰—Y—;
R¹ is selected from the group consisting of hydrogen, alkyl, alkylhalo, alkenyl, alkenylhalo, alkynyl, alkynylhalo, cycloalkyl, heterocycloalkyl, aryl, heteroaryl, alkylene-cycloalkyl, alkenylene-cycloalkyl, alkynylene-cycloalkyl, alkylene-heterocycloalkyl, alkenylene-heterocycloalkyl, alkynylene-heterocycloalkyl, alkylene-aryl, alkenylene-aryl, alkynylene-aryl, alkylene-heteroaryl, alkenylene-heteroaryl, alkynylene-heteroaryl, alkylene-OR⁷, alkenylene-OR⁷, alkynylene-OR⁷, alkylene-NR⁸R⁹, alkenylene-NR⁸R⁹, alkynylene-NR⁸R⁹, alkylene-cyano, alkenylene-cyano, alkynylene-cyano, alkylene-(CO)R⁷, alkenylene-(CO)R⁷, and alkynylene-(CO)R⁷; wherein any cyclic group is optionally substituted by one or more substituents independently selected from the group consisting of halogen, alkyl, —O-alkyl, alkylhalo, and —O-alkylhalo;
R², in each instance, is independently selected from the group consisting of hydrogen, halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, and —O-alkynylene-heteroaryl; wherein any cyclic group is optionally substituted by one or more substituents independently selected from the group consisting of halogen, alkyl, —O-alkyl, alkylhalo, and —O-alkylhalo;
R³ is selected from the group consisting of hydrogen, aryl, heteroaryl, and benzo-cycloC₅₋₈alkenyl; wherein any carbocyclic group is optionally substituted by one or more independently selected substituents, R⁵, and any heterocyclic group is optionally substituted by one or more independently selected substituents, R⁶;
R⁴, in each instance, is independently selected from the group consisting of hydrogen, halogen, hydroxyl, cyano, oxo, ═CR⁷R⁸, alkyl, alkylhalo, —O-alkyl, —O-alkylhalo, alkenyl, —O-alkenyl, alkynyl, —O-alkynyl, cycloalkyl, alkylene-cyclcoalkyl, heterocyloalkyl, alkylene-heterocycloalkyl, aryl, alkylene-aryl, heteroaryl, and alkylene-heteroaryl; wherein any cyclic group may be substituted by one or more substituents independently selected from the group consisting of halogen, alkyl, —O-alkyl, alkylhalo, and —O-alkylhalo;
R⁵, in each instance, is independently selected from the group consisting of halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, —O-alkynylene-heteroaryl, alkylene-cyano, —O-alkylene-cyano, alkenylene-cyano, —O-alkenylene-cyano, alkynylene-cyano, and —O-alkynylene-cyano; wherein any cyclic group is optionally substituted by one or more substituents independently selected from the group consisting of halogen, alkyl, —O-alkyl, alkylhalo, and —O-alkylhalo;
R⁶, in each instance, is independently selected from the group consisting of halogen, amino, cyano, alkyl, alkylhalo, alkenyl, alkynyl, and aryl; wherein said aryl is optionally substituted by one or more substituents independently selected from the group consisting of halogen, alkyl, —O-alkyl, alkylhalo, and —O-alkylhalo;
R⁷, R⁸, and R⁹ are independently selected from the group consisting of hydrogen, alkyl, alkylhalo, alkenyl, and alkynyl;
R¹⁰ is selected from the group consisting of hydrogen, alkyl, alkenyl, and alkynyl;
m represents an integer selected from the group consisting of 1, 2, 3, and 4; and n represents an integer selected from the group consisting of 1 and 2;
in the manufacture of a medicament for the therapy of neurological and psychiatric disorders associated with glutamate dysfunction.

2. The use according to claim 1 wherein D is a methylene group.

3. The use according to claim 2 wherein L is selected from the group consisting of alkylene and alkylene-O—.

4. The use according to claim 3 wherein B is C.

5. The use according to claim 1 wherein the compound selected from the group consisting of
2-{4-[2-(4-Fluoro-phenoxy)-ethyl]-piperidin-1-ylmethyl}-1-methyl-1H-benzoimidazole;
2-{4-[2-(3,4-Difluoro-phenoxy)-ethyl]-piperidin-1-ylmethyl}-1,7-dimethyl-1H-benzoimidazole;
2-{4-[2-(3,4-Difluoro-phenoxy)-ethyl]-piperidin-1-ylmethyl}-1-methyl-1H-benzoimidazole;
2-{4-[2-(4-Fluoro phenoxy)-ethyl]-piperidin-1-ylmethyl}-1,7-dimethyl-1H-benzoimidazole;
2-{4-[2-(3,4-Dichloro-phenoxy)-ethyl]-piperidin-1-ylmethyl}-1-methyl-1H-benzoimidazole;
2-{3-[3-(4-Fluoro-phenyl)-propyl]-piperidin-1-ylmethyl}-1,7-dimethyl-1H-benzoimidazole;
4-(1,7-Dimethyl-1H-benzoimidazol-2-ylmethyl)-1-(3-phenyl-propyl)-piperazin-2-one;
2-{4-[3-(3-Fluoro-5-trifluoromethyl-phenyl)-propyl]-piperidin-1-ylmethyl}-1,7-dimethyl-1H-benzoimidazole;
2-{4-[3-(4-Fluoro-phenyl)-propyl]-piperidin-1-ylmethyl}-1,7-dimethyl-1H-benzoimidazole;
2-{4-[3-(4-Fluoro-phenyl)-propyl]-piperidin-1-ylmethyl}-1-methyl-1H-benzoimidazole;
2-{4-[3-(2-Difluoromethoxy-phenyl)-propyl]-piperidin-1-ylmethyl}-1-methyl-1H-benzoimidazole;
2-{4-[3-(3-Fluoro-5-trifluoromethyl-phenyl)-propyl]-piperidin-1-ylmethyl}-1-methyl-1H-benzoimidazole;
1-Methyl-2-{4-[3-(2-trifluoromethoxy-phenyl)-propyl]-piperidin-1-ylmethyl}-1H-benzoimidazole;
1-Isopropyl-2-{4-[3-(3-methoxy-phenyl)-propyl]-piperidin-1-ylmethyl}-1H-benzoimidazole;
1-Isopropyl-2-{4-[3-(2-methoxy-phenyl)-propyl]-piperidin-1-ylmethyl}-1H-benzoimidazole;
2-{4-[3-(4-Methoxy-phenyl)-propyl]-piperidin-1-ylmethyl}-1-methyl-1H-benzoimidazole;
2-{4-[3-(3-Methoxy-phenyl)-propyl]-piperidin-1-ylmethyl}-1-methyl-1H-benzoimidazole;
2-{4-[3-(2-Methoxy-phenyl)-propyl]-piperidin-1-ylmethyl}-1-methyl-1H-benzoimidazole;
2-{3-[1-(1-Methyl-1H-benzoimidazol-2-ylmethyl)-piperidin-4-yl]-propyl}-benzonitrile;
3-{3-[1-(1-Methyl-1H-benzoimidazol-2-ylmethyl)-piperidin-4-yl]-propyl}-benzonitrile;
7-Chloro-1-methyl-2-[4-(3-phenyl-propyl)-piperidin-1-ylmethyl]-1H-benzoimidazole;
1,6-Dimethyl-2-[4-(3-phenyl-propyl)-piperidin-1-ylmethyl]-1H-benzoimidazole;
4-Chloro-1-methyl-2-[4-(3-phenyl-propyl)-piperidin-1-ylmethyl]-1H-benzoimidazole;
1-Cyclopropyl-2-[4-(3-phenyl-propyl)-piperidin-1-ylmethyl]-1H-benzoimidazole;
6-Chloro-1-methyl-2-[4-(3-phenyl-propyl)-piperidin-1-ylmethyl]-1H-benzoimidazole;
1-Ethyl-2-[4-(3-phenyl-propyl)-piperidin-1-ylmethyl]-1H-benzoimidazole;
1,7-Dimethyl-2-[4-(3-phenyl-propyl)-piperidin-1-ylmethyl]-1H-benzoimidazole;
1,5-Dimethyl-2-[4-(3-phenyl-propyl)-piperidin-1-ylmethyl]-1H-benzoimidazole;
1-Isopropyl-2-[4-(3-phenyl-propyl)-piperidin-1-ylmethyl]-1H-benzoimidazole;
2-{4-[2-(4-Fluoro-phenyl)-ethyl]-piperidin-1-ylmethyl}-1,7-dimethyl-1H-benzoimidazole;
2-{3-[2-(4-Fluoro-phenyl)-ethyl]-pyrrolidin-1-ylmethyl}-1,7-dimethyl-1H-benzoimidazole;
2-{3-[2-(4-Fluoro-phenyl)-ethyl]-pyrrolidin-1-ylmethyl}-1-methyl-1H-benzoimidazole;
2-{3-[2-(4-Fluoro-phenyl)-ethyl]-piperidin-1-ylmethyl}-1,7-dimethyl-1H-benzoimidazole;
7-Chloro-2-[4-(4-fluoro-phenyl)-piperazin-1-ylmethyl]-1-methyl-1H-benzoimidazole;
1-Ethyl-2-[4-(4-fluoro-phenyl)-piperazin-1-ylmethyl]-1H-benzoimidazole;
2-[4-(4-Fluoro-phenyl)-piperazin-1-ylmethyl]-1,6-dimethyl-1H-benzoimidazole;
5-Chloro-2-[4-(4-fluoro-phenyl)-piperazin-1-ylmethyl]-1-methyl-1H-benzoimidazole;
2-[4-(4-Fluoro-phenyl)-piperazin-1-ylmethyl]-1,7-dimethyl-1H-benzoimidazole;
2-[4-(4-Fluoro-phenyl)-piperazin-1-ylmethyl]-1,5-dimethyl-1H-benzoimidazole;
1,6-Dimethyl-2-(4-phenyl-piperidin-1-ylmethyl)-1H-benzoimidazole;
2-[4-(4-Fluoro-phenyl)-piperidin-1-ylmethyl]-1-methyl-1H-benzoimidazole;
2-[4-(4-Chloro-phenyl)-piperidin-1-ylmethyl]-1-methyl-1H-benzoimidazole;
1-Methyl-2-[4-(4-trifluoromethyl-phenyl)-piperidin-1-ylmethyl]-1H-benzoimidazole;
1,7-Dimethyl-2-[4-(3-trifluoromethyl-1-phenyl)-piperidin-1-ylmethyl]-1H-benzoimidazole;
1,7-Dimethyl-2-[4-(2-trifluoromethyl-1-phenyl)-piperidin-1-yl methyl]-1H-benzoimidazole;
2-[4-(2-Fluoro-phenyl)-piperidin-1-ylmethyl]-1,7-dimethyl-1H-benzoimidazole;
2-[4-(3-Fluoro-phenyl)-piperidin-1-ylmethyl]-1,7-dimethyl-1H-benzoimidazole;
1,7-Dimethyl-2-[4-(4-trifluoromethyl-phenyl)-piperidin-1-ylmethyl]-1H-benzoimidazole;
2-[4-(4-Fluoro-phenyl)-piperidin-1-ylmethyl]-1,7-dimethyl-1H-benzoimidazole;
1-(1-Methyl-1H-benzoimidazol-2-ylmethyl)-4-phenyl-piperidine-4-carbonitrile;
5-Chloro-2-(4,4-diphenyl-piperidin-1-ylmethyl)-1-methyl-1H-benzoimidazole;
7-Chloro-2-(4,4-diphenyl-piperidin-1-ylmethyl)-1-methyl-1H-benzoimidazole;
2-(4,4-Diphenyl-piperidin-1-ylmethyl)-1,7-dimethyl-1H-benzoimidazole;
2-(4,4-Diphenyl-piperidin-1-ylmethyl)-1-ethyl-1H-benzoimidazole;
1-Cyclopropyl-2-(4,4-diphenyl-piperidin-1-ylmethyl)-1H-benzoimidazole;
2-(4,4-Diphenyl-piperidin-1-ylmethyl)-1-isopropyl-1H-benzoimidazole;
7-Chloro-1-methyl-2-(4-phenyl-piperidin-1-ylmethyl)-1H-benzoimidazole;
4-Chloro-1-methyl-2-(4-phenyl-piperidin-1-ylmethyl)-1H-benzoimidazole;
5-Chloro-1-methyl-2-(4-phenyl-piperidin-1-ylmethyl)-1H-benzoimidazole;
6-Chloro-1-methyl-2-(4-phenyl-piperidin-1-ylmethyl)-1H-benzoimidazole;
1-Ethyl-2-(4-phenyl-piperidin-1-ylmethyl)-1H-benzoimidazole;
1,7-Dimethyl-2-(4-phenyl-piperidin-1-ylmethyl)-1H-benzoimidazole;
1,5-Dimethyl-2-(4-phenyl-piperidin-1-ylmethyl)-1H-benzoimidazole;
1-Isopropyl-2-(4-phenyl-piperidin-1-ylmethyl)-1H-benzoimidazole;
2-(4-Allyl-piperidin-1-ylmethyl)-1-methyl-1H-benzoimidazole;
1-Methyl-2-(4-methylene-piperidin-1-ylmethyl)-1H-benzoimidazole;
2-[3-(4-Fluoro-benzyl)-piperidin-1-ylmethyl]-1,7-dimethyl-1H-benzoimidazole;
2-[3-(4-Fluoro-benzyl)-piperidin-1-ylmethyl]-1-methyl-1H-benzoimidazole;
2-{4-[2-(4-Chloro-phenoxy)-ethyl]-piperidin-1-ylmethyl}-1-methyl-1H-benzoimidazole;
2-(4-Phenyl-piperidin-1-ylmethyl)-1-propyl-1H-benzoimidazole;
2-[4-(3-Phenyl-propyl)-piperidin-1-ylmethyl]-1-propyl-1H-benzoimidazole;
2-[4-(4-Fluoro-phenyl)-piperzin-1-ylmethyl]-1-isopropyl-1H-benzoimidazole;
3-{3-[1-(1-Methyl-1H-benzoimidazol-2-ylmethyl)-piperidin-4-yl]-propyl}-pyridine-2-carbonitrile;
4-(4-Bromo-phenyl)-1-(1-methyl-1H-benzoimidazol-2-ylmethyl)-piperidin-4-ol;
4-(4-Chloro-phenyl)-1-(1-methyl-1H-benzoimidazol-2-ylmethyl)-piperidin-4-ol;
2-(4,4-Diphenyl-piperidin-1-ylmethyl)-1,5-dimethyl-1H-benzoimidazole;
1-Methyl-2-[4-(3-phenyl-propyl)-piperazin-1-ylmethyl]-1H-benzoimidazole;
5-Chloro-1-methyl-2-[4-(3-phenyl-propyl)-piperidin-1-ylmethyl]-1H-benzoimidazole;
6-Chloro-2-(4,4-diphenyl-piperidin-1-ylmethyl)-1-methyl-1H-benzoimidazole;
1-Cyclopropyl-2-(4-phenyl-piperidin-1-ylmethyl)-1H-benzoimidazole;
2-{3-[3-(4-Fluoro-phenyl)-propyl]-piperidin-1-ylmethyl}-1-methyl-1H-benzoimidazole;
2-{3-[2-(4-Fluoro-phenyl)-ethyl]-piperidin-1-ylmethyl}-1-methyl-1H-benzoimidazole;
6-Chloro-2-[4-(4-fluoro-phenyl)-piperazin-1-ylmethyl]-1-methyl-1H-benzoimidazole;
2-(4,4-Diphenyl-piperidin-1-ylmethyl)-1,6-dimethyl-1H-benzoimidazole;
2-{3-[4-(4-Fluoro-phenyl)-piperidin-1-yl]-propyl}-1,7-dimethyl-1H-benzoimidazole;
2-{3-[4-(3-Fluoro-phenyl)-piperidin-1-yl]-propyl}-1,7-dimethyl-1H-benzoimidazole;
2-(3-{4-[2-(4-Fluoro-phenoxy)-ethyl]-piperidin-1-yl}-propyl)-1,7-dimethyl-1H-benzoimidazole;
2-[1-(4-Fluoro-benzyl)-piperidin-4-ylmethyl]-1,7-dimethyl-1H-benzoimidazole;
2-[1-(4-Fluoro-phenyl)-piperidin-4-ylmethyl]-1,7-dimethyl-1H-benzoimidazole;
tert-Butyl 4-[(1-methyl-7-methyl-1H-benzimidazol-2-yl)methyl]piperidine-1-carboxylate;
1,7-Dimethyl-2-piperidin-4-ylmethyl-1H-benzoimidazole;
2-[1-(4-Benzyl-piperidin-1-yl)-ethyl]-1-methyl-1H-benzoimidazole;
2-(4-Benzyl-piperidin-1-ylmethyl)-1-(4-bromo-benzyl)-1H-benzoimidazole;
2-(4-Benzyl-piperidin-1-ylmethyl)-1-(4-chloro-benzyl)-1H-benzoimidazole;
1-Methyl-2-(3-phenyl-propoxymethyl)-1H-benzoimidazole;
2-[4-(2-Fluoro-phenyl)-piperazin-1-ylmethyl]-1-methyl-1H-benzoimidazole;
2-[4-(4-Fluoro-phenyl)-piperazin-1-ylmethyl]-1-methyl-1H-benzoimidazole;
1-Methyl-2-(4-m-tolyl-piperazin-1-ylmethyl)-1H-benzoimidazole;
2-[4-(3,4-Dichloro-phenyl)-piperazin-1-ylmethyl]-1-methyl-1H-benzoimidazole;
2-[4-(4-Methoxy-phenyl)-piperazin-1-ylmethyl]-1-methyl-1H-benzoimidazole;
1-Methyl-2-(4-p-tolyl-piperazin-1-ylmethyl)-1H-benzoimidazole;
2-[4-(3-Chloro-phenyl)-piperazin-1-ylmethyl]-1-methyl-1H-benzoimidazole;
2-[4-(4-Chloro-phenyl)-piperazin-1-ylmethyl]-1-methyl-1H-benzoimidazole;
2-(4,4-Diphenyl-piperidin-1-ylmethyl)-1-methyl-1H-benzoimidazole;
2-(4-Benzyl-piperidin-1-ylmethyl)-1-methyl-1H-benzoimidazole;
1-Methyl-2-(4-phenyl-piperidin-1-ylmethyl)-1H-benzoimidazole;
1-Methyl-2-(4-o-tolyl-piperazin-1-ylmethyl)-1H-benzoimidazole;
2-[4-(2-Methoxy-phenyl)-piperazin-1-ylmethyl]-1-methyl-1H-benzoimidazole;
2-(4-Benzyl-piperazin-1-ylmethyl)-1-methyl-1H-benzoimidazole;
1-Methyl-2-piperidin-1-ylmethyl-1H-benzoimidazole;
1-Methyl-2-(4-phenyl-piperazin-1 ylmethyl)-1H-benzoimidazole;
1-Methyl-2-(4-pyrimidin-2-yl-piperazin-1-ylmethyl)-1H-benzoimidazole;
2-[4-(2-Chloro-phenyl)-piperazin-1 ylmethyl]-1-methyl-1H-benzoimidazole;
1-Allyl-2-(4-o-tolyl-piperazin-1 ylmethyl)-1H-benzoimidazole;
1-Benzyl-2-(4-o-tolyl-piperazin-1 ylmethyl)-1H-benzoimidazole;
(S)-1-Methyl-2-[4-(1,2,3,4-tetrahydro-naphthalen-1-yl)-[1,4]diazepan-1-ylmethyl]-1H-benzoimidazole; and
2-(4-Benzyl-piperidin-1-ylmethyl)-1-(4-trifluoromethoxy-benzyl)-1H-benzoimidazole.

6. The use of claim 1, wherein the neurological and psychiatric disorders are selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, post-traumatic pain, tardive dyskinesia, sleep disorders, narcolepsy, attention deficit/hyperactivity disorder, and conduct disorder.

7. A method for the treatment or prevention of neurological and psychiatric disorders associated with glutamate dysfunction in an animal in need of such treatment, comprising the step of administering to said animal a therapeutically effective amount of a compound of Formula I: 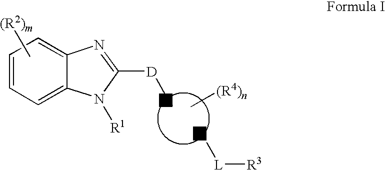
wherein,
A and B are independently selected from the group consisting of N and C, with the proviso that A and B are not both C; 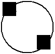
represents a 4- to 8-membered ring;
D is selected from the group consisting of alkylene, alkenylene, and alkynylene;
L is selected from the group consisting of a bond, alkylene, alkenylene, alkynylene, —O—, —X—O—, —O—X—, —X—O—Y, —NR¹⁰—, —X—NR¹⁰—, —NR¹⁰—X—, and —X—NR¹⁰—Y—; wherein X and Y, in each instance, are independently selected from the group consisting of alkylene, alkenylene, and alkynylene, with the proviso that when B is N, L is selected from the group consisting of a bond, alkylene, alkenylene, alkynylene, —X—O—, —X—O—Y—, —X—NR¹⁰—, and —X—NR¹⁰—Y—;
R¹ is selected from the group consisting of hydrogen, alkyl, alkylhalo, alkenyl, alkenylhalo, alkynyl, alkynylhalo, cycloalkyl, heterocycloalkyl, aryl, heteroaryl, alkylene-cycloalkyl, alkenylene-cycloalkyl, alkynylene-cycloalkyl, alkylene-heterocycloalkyl, alkenylene-heterocycloalkyl, alkynylene-heterocycloalkyl, alkylene-aryl, alkenylene-aryl, alkynylene-aryl, alkylene-heteroaryl, alkenylene-heteroaryl, alkynylene-heteroaryl, alkylene-OR⁷, alkenylene-OR⁷, alkynylene-OR⁷, alkylene-NR⁸R⁹, alkenylene-NR⁸R⁹, alkynylene-NR⁸R⁹, alkylene-cyano, alkenylene-cyano, alkynylene-cyano, alkylene-(CO)R⁷, alkenylene-(CO)R⁷, and alkynylene-(CO)R⁷; wherein any cyclic group is optionally substituted by one or more substituents independently selected from the group consisting of halogen, alkyl, —O-alkyl, alkylhalo, and —O-alkylhalo;
R², in each instance, is independently selected from the group consisting of hydrogen, halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, and —O-alkynylene-heteroaryl; wherein any cyclic group is optionally substituted by one or more substituents independently selected from the group consisting of halogen, alkyl, —O-alkyl, alkylhalo, and —O-alkylhalo;
R³ is selected from the group consisting of hydrogen, aryl, heteroaryl, and benzo-cycloC₅₋₈alkenyl; wherein any carbocyclic group is optionally substituted by one or more independently selected substituents, R⁵, and any heterocyclic group is optionally substituted by one or more independently selected substituents, R;
R⁴, in each instance, is independently selected from the group consisting of hydrogen, halogen, hydroxyl, cyano, oxo, ═CR⁷R⁸, alkyl, alkylhalo, —O-alkyl, —O-alkylhalo, alkenyl, —O-alkenyl, alkynyl, —O-alkynyl, cycloalkyl, alkylene-cyclcoalkyl, heterocyloalkyl, alkylene-heterocycloalkyl, aryl, alkylene-aryl, heteroaryl, and alkylene-heteroaryl; wherein any cyclic group may be substituted by one or more substituents independently selected from the group consisting of halogen, alkyl, —O-alkyl, alkylhalo, and —O-alkylhalo;
R⁵, in each instance, is independently selected from the group consisting of halogen, cyano, alkyl, —O-alkyl, alkylhalo, —O-alkylhalo, alkenyl, —O-alkenyl, alkynyl, —O-alkynyl, cycloalkyl, heterocyloalkyl, aryl, heteroaryl, alkylene-cycloalkyl, alkenylene-cycloalkyl, alkynylene-cycloalkyl, —O-alkylene-cycloalkyl, —O-alkenylene-cycloalkyl, —O-alkynylene-cycloalkyl, alkylene-heterocycloalkyl, alkenylene-heterocycloalkyl, alkynylene-heterocycloalkyl, —O-alkylene-heterocycloalkyl, —O-alkenylene-heterocycloalkyl, —O-alkynylene-heterocycloalkyl, alkylene-aryl, alkenylene-aryl, alkynylene-aryl, —O-alkylene-aryl, —O-alkenylene-aryl, —O-alkynylene-aryl, alkylene-heteroaryl, alkenylene-heteroaryl, alkynylene-heteroaryl, —O-alkylene-heteroaryl, —O-alkenylene-heteroaryl, —O-alkynylene-heteroaryl, alkylene-cyano, —O-alkylene-cyano, alkenylene-cyano, —O-alkenylene-cyano, alkynylene-cyano, and —O-alkynylene-cyano; wherein any cyclic group is optionally substituted by one or more substituents independently selected from the group consisting of halogen, alkyl, —O-alkyl, alkylhalo, and —O-alkylhalo;
R⁶, in each instance, is independently selected from the group consisting of halogen, amino, cyano, alkyl, alkylhalo, alkenyl, alkynyl, and aryl; wherein said aryl is optionally substituted by one or more substituents independently selected from the group consisting of halogen, alkyl, —O-alkyl, alkylhalo, and —O-alkylhalo;
R⁷, R⁸, and R⁹ are independently selected from the group consisting of hydrogen, alkyl, alkylhalo, alkenyl, and alkynyl;
R¹⁰ is selected from the group consisting of hydrogen, alkyl, alkenyl, and alkynyl;
m represents an integer selected from the group consisting of 1, 2, 3, and 4; and n represents an integer selected from the group consisting of 1 and 2.

8. The method according to claim 7, wherein the neurological and psychiatric disorders are selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, post-traumatic pain, tardive dyskinesia, sleep disorders, narcolepsy, attention deficit/hyperactivity disorder, and conduct disorder.

9. The method according to claim 8, wherein the neurological and psychiatric disorders are selected from Alzheimer's disease, cerebral deficits secondary to prolonged status epilepticus, substance tolerance, substance withdrawal, psychosis, schizophrenia, anxiety, generalized anxiety disorder, panic disorder, social phobia, obsessive compulsive disorder, post-traumatic stress disorder (PTSD), mood disorders, depression, mania, and bipolar disorders.

10. A compound according to Formula II: 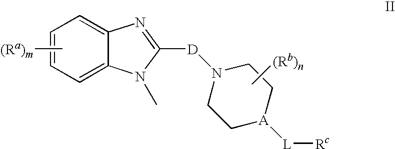
wherein:
A is selected from the group consisting of C and N;
D is an alkylene group;
L is selected from the group consisting of a bond, alkylene, alkylene-O—, —O-alkylene and alkylene-O-alkylene;
R^{a}, in each instance, is independently selected from the group consisting of halo and alkyl;
R^{b}, in each instance, is independently selected from the group consisting of halogen, cyano, oxo, hydroxy, alkyl, alkylhalo, —O-alkyl and —O-alkylhalo;
R^{c} is selected from the group consisting of aryl and heteroaryl, optionally substituted by one or more substituents independently selected from the group consisting of halo, cyano, hydroxy, alkyl, O-alkyl, alkylhalo, O-alkylhalo; and
m and n are independently selected from the group consisting of 0, 1, 2 and 3.

11. A compound according to claim 10 wherein D is methylene.

12. A compound according to claim 11 wherein L is selected from the group consisting of a bond, alkylene and alkylene-O—.

13. A compound according to claim 10 selected from the group consisting of:
2-{4-[2-(4-Fluoro-phenoxy)-ethyl]-piperidin-1-ylmethyl}-1-methyl-1H-benzoimidazole;
2-{4-[2-(3,4-Difluoro-phenoxy)-ethyl]-piperidin-1-ylmethyl}-1,7-dimethyl-1H-benzoimidazole;
2-{4-[2-(3,4-Difluoro-phenoxy)-ethyl]-piperidin-1-ylmethyl}-1-methyl-1H-benzoimidazole;
2-{4-[2-(4-Fluoro phenoxy)-ethyl]-piperidin-1-ylmethyl}-1,7-dimethyl-1H-benzoimidazole;
2-{4-[2-(3,4-Dichloro-phenoxy)-ethyl]-piperidin-1-ylmethyl}-1-methyl-1H-benzoimidazole;
2-{3-[3-(4-Fluoro-phenyl)-propyl]-piperidin-1-ylmethyl}-1,7-dimethyl-1H-benzoimidazole;
4-(1,7-Dimethyl-1H-benzoimidazol-2-ylmethyl)-1-(3-phenyl-propyl)-piperazin-2-one;
2-{4-[3-(3-Fluoro-5-trifluoromethyl-phenyl)-propyl]-piperidin-1-ylmethyl}-1,7-dimethyl-1H-benzoimidazole;
2-{4-[3-(4-Fluoro-phenyl)-propyl]-piperidin-1-ylmethyl}-1,7-dimethyl-1H-benzoimidazole;
2-{4-[3-(4-Fluoro-phenyl)-propyl]-piperidin-1-ylmethyl}-1-methyl-1H-benzoimidazole;
2-{4-[3-(2-Difluoromethoxy-phenyl)-propyl]-piperidin-1-ylmethyl}-1-methyl-1H-benzoimidazole;
2-{4-[3-(3-Fluoro-5-trifluoromethyl-phenyl)-propyl]-piperidin-1-ylmethyl}-1-methyl-1H-benzoimidazole;
1-Methyl-2-{4-[3-(2-trifluoromethoxy-phenyl)-propyl]-piperidin-1-ylmethyl}-1H-benzoimidazole;
1-Isopropyl-2-{4-[3-(3-methoxy-phenyl)-propyl]-piperidin-1-ylmethyl}-1H-benzoimidazole;
1-Isopropyl-2-{4-[3-(2-methoxy-phenyl)-propyl]-piperidin-1-ylmethyl}-1H-benzoimidazole;
2-{4-[3-(4-Methoxy-phenyl)-propyl]-piperidin-1-ylmethyl}-1-methyl-1H-benzoimidazole;
2-{4-[3-(3-Methoxy-phenyl)-propyl]-piperidin-1-ylmethyl}-1-methyl-1H-benzoimidazole;
2-{4-[3-(2-Methoxy-phenyl)-propyl]-piperidin-1-ylmethyl}-1-methyl-1H-benzoimidazole;
2-{3-[1-(1-Methyl-1H-benzoimidazol-2-ylmethyl)-piperidin-4-yl]-propyl}-benzonitrile;
3-{3-[1-(1-Methyl-1H-benzoimidazol-2-ylmethyl)-piperidin-4-yl]-propyl}-benzonitrile;
7-Chloro-1-methyl-2-[4-(3-phenyl-propyl)-piperidin-1-ylmethyl]-1H-benzoimidazole;
1,6-Dimethyl-2-[4-(3-phenyl-propyl)-piperidin-1-ylmethyl]-1H-benzoimidazole;
4-Chloro-1-methyl-2-[4-(3-phenyl-propyl)-piperidin-1-ylmethyl]-1H-benzoimidazole;
6-Chloro-1-methyl-2-[4-(3-phenyl-propyl)-piperidin-1-ylmethyl]-1H-benzoimidazole;
1-Ethyl-2-[4-(3-phenyl-propyl)-piperidin-1-ylmethyl]-1H-benzoimidazole;
1,7-Dimethyl-2-[4-(3-phenyl-propyl)-piperidin-1-ylmethyl]-1H-benzoimidazole;
1,5-Dimethyl-2-[4-(3-phenyl-propyl)-piperidin-1-ylmethyl]-1H-benzoimidazole;
1-Isopropyl-2-[4-(3-phenyl-propyl)-piperidin-1-ylmethyl]-1H-benzoimidazole;
2-{4-[2-(4-Fluoro-phenyl)-ethyl]-piperidin-1-ylmethyl}-1,7-dimethyl-1H-benzoimidazole;
7-Chloro-2-[4-(4-fluoro-phenyl)-piperazin-1-ylmethyl]-1-methyl-1H-benzoimidazole;
1-Ethyl-2-[4-(4-fluoro-phenyl)-piperazin-1-ylmethyl]-1H-benzoimidazole;
2-[4-(4-Fluoro-phenyl)-piperazin-1-ylmethyl]-1,6-dimethyl-1H-benzoimidazole;
5-Chloro-2-[4-(4-fluoro-phenyl)-piperazin-1-ylmethyl]-1-methyl-1H-benzoimidazole;
2-[4-(4-Fluoro-phenyl)-piperazin-1-ylmethyl]-1,7-dimethyl-1H-benzoimidazole;
2-[4-(4-Fluoro-phenyl)-piperazin-1-ylmethyl]-1,5-dimethyl-1H-benzoimidazole;
1,6-Dimethyl-2-(4-phenyl-piperidin-1-ylmethyl)-1H-benzoimidazole;
2-[4-(4-Fluoro-phenyl)-piperidin-1-ylmethyl]-1-methyl-1H-benzoimidazole;
2-[4-(4-Chloro-phenyl)-piperidin-1-ylmethyl]-1-methyl-1H-benzoimidazole;
1-Methyl-2-[4-(4-trifluoromethyl-phenyl)-piperidin-1-ylmethyl]-1H-benzoimidazole;
1,7-Dimethyl-2-[4-(3-trifluoromethyl-1-phenyl)-piperidin-1-ylmethyl]-1H-benzoimidazole;
1,7-Dimethyl-2-[4-(2-trifluoromethyl-1-phenyl)-piperidin-1-yl methyl]-1H-benzoimidazole;
2-[4-(2-Fluoro-phenyl)-piperidin-1-ylmethyl]-1,7-dimethyl-1H-benzoimidazole;
2-[4-(3-Fluoro-phenyl)-piperidin-1-ylmethyl]-1,7-dimethyl-1H-benzoimidazole;
1,7-Dimethyl-2-[4-(4-trifluoromethyl-phenyl)-piperidin-1-ylmethyl]-1H-benzoimidazole;
2-[4-(4-Fluoro-phenyl)-piperidin-1-ylmethyl]-1,7-dimethyl-1H-benzoimidazole;
1-(1-Methyl-1H-benzoimidazol-2-ylmethyl)-4-phenyl-piperidine-4-carbonitrile;
7-Chloro-1-methyl-2-(4-phenyl-piperidin-1-ylmethyl)-1H-benzoimidazole;
1-Ethyl-2-(4-phenyl-piperidin-1-ylmethyl)-1H-benzoimidazole;
1,7-Dimethyl-2-(4-phenyl-piperidin-1-ylmethyl)-1H-benzoimidazole;
1-Isopropyl-2-(4-phenyl-piperidin-1-ylmethyl)-1H-benzoimidazole;
2-{4-[2-(4-Chloro-phenoxy)-ethyl]-piperidin-1-ylmethyl}-1-methyl-1H-benzoimidazole;
2-(4-Phenyl-piperidin-1-ylmethyl)-1-propyl-1H-benzoimidazole;
2-[4-(3-Phenyl-propyl)-piperidin-1-ylmethyl]-1-propyl-1H-benzoimidazole;
2-[4-(4-Fluoro-phenyl)-piperzin-1-ylmethyl]-1-isopropyl-1H-benzoimidazole;
3-{3-[1-(1-Methyl-1H-benzoimidazol-2-ylmethyl)-piperidin-4-yl]-propyl}-pyridine-2-carbonitrile;
4-(4-Bromo-phenyl)-1-(1-methyl-1H-benzoimidazol-2-ylmethyl)-piperidin-4-ol;
4-(4-Chloro-phenyl)-1-(1-methyl-1H-benzoimidazol-2-ylmethyl)-piperidin-4-ol;
1-Methyl-2-[4-(3-phenyl-propyl)-piperazin-1-ylmethyl]-1H-benzoimidazole;
2-{3-[4-(4-Fluoro-phenyl)-piperidin-1-yl]-propyl}-1,7-dimethyl-1H-benzoimidazole;
2-{3-[4-(3-Fluoro-phenyl)-piperidin-1-yl]-propyl}-1,7-dimethyl-1H-benzoimidazole;
2-(3-{4-[2-(4-Fluoro-phenoxy)-ethyl]-piperidin-1-yl}-propyl)-1,7-dimethyl-1H-benzoimidazole;
2-[1-(4-Fluoro-benzyl)-piperidin-4-ylmethyl]-1,7-dimethyl-1H-benzoimidazole;
2-[1-(4-Fluoro-phenyl)-piperidin-4-ylmethyl]-1,7-dimethyl-1H-benzoimidazole;
2-[1-(4-Benzyl-piperidin-1-yl)-ethyl]-1-methyl-1H-benzoimidazole;
2-[4-(2-Fluoro-phenyl)-piperazin-1-ylmethyl]-1-methyl-1H-benzoimidazole;
2-[4-(4-Fluoro-phenyl)-piperazin-1-ylmethyl]-1-methyl-1H-benzoimidazole;
1-Methyl-2-(4-m-tolyl-piperazin-1-ylmethyl)-1H-benzoimidazole;
2-[4-(3,4-Dichloro-phenyl)-piperazin-1-ylmethyl]-1-methyl-1H-benzoimidazole;
2-[4-(4-Methoxy-phenyl)-piperazin-1-ylmethyl]-1-methyl-1H-benzoimidazole;
1-Methyl-2-(4-p-tolyl-piperazin-1-ylmethyl)-1H-benzoimidazole;
2-[4-(3-Chloro-phenyl)-piperazin-1-ylmethyl]-1-methyl-1H-benzoimidazole;
2-[4-(4-Chloro-phenyl)-piperazin-1-ylmethyl]-1-methyl-1H-benzoimidazole;
2-(4-Benzyl-piperidin-1-ylmethyl)-1-methyl-1H-benzoimidazole;
1-Methyl-2-(4-phenyl-piperidin-1-ylmethyl)-1H-benzoimidazole;
1-Methyl-2-(4-o-tolyl-piperazin-1-ylmethyl)-1H-benzoimidazole;
2-[4-(2-Methoxy-phenyl)-piperazin-1-ylmethyl]-1-methyl-1H-benzoimidazole;
2-(4-Benzyl-piperazin-1-ylmethyl)-1-methyl-1H-benzoimidazole;
1-Methyl-2-(4-phenyl-piperazin-1 ylmethyl)-1H-benzoimidazole;
1-Methyl-2-(4-pyrimidin-2-yl-piperazin-1-ylmethyl)-1H-benzoimidazole and 2-[4-(2-Chloro-phenyl)-piperazin-1 ylmethyl]-1-methyl-1H-benzoimidazole.

14. A pharmaceutical composition comprising a compound according to any one of claims 10-13 and a pharmaceutically acceptable carrier or excipient.

15. A compound according to any one of claims 10-13 for use as a medicament.

16. A use of a compound according to any one of claims 10-13 in the manufacture of a medicament for the therapy of neurological and psychiatric disorders associated with glutamate dysfunction.

17. The use of claim 16, wherein the neurological and psychiatric disorders are selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, post-traumatic pain, tardive dyskinesia, sleep disorders, narcolepsy, attention deficit/hyperactivity disorder, and conduct disorder.

18. A method for the treatment or prevention of neurological and psychiatric disorders associated with glutamate dysfunction in an animal in need of such treatment, comprising the step of administering to said animal a therapeutically effective amount of a compound according to any one of claims 10-13.

19. A method for the treatment or prevention of neurological and psychiatric disorders associated with glutamate dysfunction in an animal in need of such treatment, comprising the step of administering to said animal a therapeutically effective amount of a pharmaceutical composition according to claim 14.

20. The method according to claim 18, wherein the neurological and psychiatric disorders are selected from cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine, migraine headache, urinary incontinence, substance tolerance, substance withdrawal, psychosis, schizophrenia, anxiety, generalized anxiety disorder, panic disorder, social phobia, obsessive compulsive disorder, and post-traumatic stress disorder (PTSD), mood disorders, depression, mania, bipolar disorders, circadian rhythm disorders, jet lag, shift work, trigeminal neuralgia, hearing loss, tinnitus, macular degeneration of the eye, emesis, brain edema, pain, acute pain, chronic pain, severe pain, intractable pain, neuropathic pain, inflammatory pain, post-traumatic pain, tardive dyskinesia, sleep disorders, narcolepsy, attention deficit/hyperactivity disorder, and conduct disorder.

21. The method according to claim 20, wherein the neurological and psychiatric disorders are selected from Alzheimer's disease, cerebral deficits secondary to prolonged status epilepticus, substance tolerance, substance withdrawal, psychosis, schizophrenia, anxiety, generalized anxiety disorder, panic disorder, social phobia, obsessive compulsive disorder, post-traumatic stress disorder (PTSD), mood disorders, depression, mania, and bipolar disorders.
